SEQUENCE LISTING (36 sequences)

SEQ ID NO: 1 - protein, 712 aa, Leptographium truncatum
MQKDTKFLNKSNIFIKNINNKYKLIPFNIKINFVGENKYFPSDFKEWTNNIYYFNSNYIKNFPVYDLNLNKLLKGYFDLYFNRESIQSKFKFFRKRRLSLNKIFVSKPEIKHTSSKTTITVYVYNRERIVLLKKLIKLRKSLFKINNFFYKCKSISGDLYGKYFINVLYKELVYIRRCKLKLNLNELKFKDQFLHKLSLLISKLYNKKVEFNIINLKSIVYNSSIFTEIMGKKLRNKNTSLLKTMKFILSKGIILEENNKKERSRLIKSVNFSLLENKYKNLNINSFVKDIDLNETIKDLYNIESKDNKDIVFDSIKYKNIGGIRLEAKGRLTKRYRADRALFKVNWKGGLKNTDSSYKGLSSVNFRGNLKSNVEYSMGISKRRIGAFAVKGWISGKSYSTLANFPVQARNDNISPWTITGFADAESSFMLTVSKDSKRNTGWSVRPRFRIGLHNKDVTILKSIREYLGAGIITSDKDARIRFESLKELEVVINHFDKYPLITQKRADYLLFKKAFYLIKNKEHLTEEGLNQILTLKASLNLGLSEELKEAFPNTIPAEKLLVTGQEIPDSNWVAGFTAGEGSFYIRIAKNSTLKTGYQVQSVFQITQDTRDIELMKNLISYLNCGNIRIRKYKGSEGIHDTCVDLVVTNLNDIKEKIIPFFNKNHIIGVKLQDYRDWCKVVTLIDNKEHLTSEGLEKIQKIKEGMNRGRSL

SEQ ID NO: 2 - DNA, 2548 bp, Leptographium truncatum
aattgatatacatattgggttaatttcaagaattacttatattgtaataagtaaatttgaagcgaagtttattttaacctttcgtccccgaccatacgacccactatttaggggtgtttaatatttagggcgcgagctcttaagagctccgcgcttaaaaactaaaaagactaggtcgagggattaaaaaagaaataaaattgttcaacgactaagagtgagttatataacaataatctcttattaatacccaacattttttttattattgtaagttagtttttataactataaaagaataaatgcaaaaagatacaaaatttttaaataaaagtaatatatttattaaaaatattaataataaatataaattaataccgttcaatattaaaattaactttgtaggtgaaaataaatattttccttctgactttaaagaatgaactaataatatctattattttaattctaattatattaaaaattttcctgtatatgatttaaatttaaataaattattaaaaggttattttgatttatattttaatcgtgaaagtattcaatctaaatttaaattttttagaaaaagacgtttatctttaaacaaaatatttgtaagtaaacctgaaataaaacatactagctctaaaactacaattacagtgtatgtttataatagagaaagaattgtcttattaaaaaaattaattaaactaagaaagtcattatttaaaataaataatttcttttataaatgtaaaagtatttctggggatctgtatggtaaatattttataaatgttttatacaaagaattagtttatataagaagatgtaaattaaaacttaatcttaatgaattaaaatttaaagatcaatttttacataaattaagtctattaataagtaaattatataataaaaaagtagagtttaacattattaatttaaaatctattgtttataattctagtatttttacagaaataatgggtaaaaaattaagaaataaaaatacaagtcttttaaaaacaatgaaatttattttaagtaaaggtattattttagaggaaaataataaaaaagaaagaagtagattaattaaaagtgtaaatttcagtcttttagagaataaatataaaaatcttaatattaattctttcgttaaagatatagatctaaatgaaacaataaaagatttatataatattgaaagtaaagataataaagatattgtatttgattctattaaatacaaaaacataggaggtataagattagaagctaaaggtagattaactaagcgttatagagcagatagagcattatttaaagttaattgaaaaggaggattaaaaaatacagattcatcttataaaggattgtcatccgtaaactttagaggaaatcttaagtctaatgtggaatattcaatgggcatatctaaacgtagaataggagcttttgctgttaaaggttgaatatcaggtaaatcttatagtacattggctaattttccggtgcaagctagaaatgacaatattagtccttgaactattacaggatttgcagacgctgaaagttcttttatgcttactgttagtaaagatagtaaacgtaatacaggatgatcagttagaccacgttttcgtataggtttacataataaagatgtaactatattaaaaagtattcgtgaatatttaggtgcaggaataattacttctgataaagatgctagaataagatttgaatctttaaaagaattggaggtagttataaatcattttgataaatatccattaataactcaaaaacgtgcggactatcttctttttaaaaaagcattttatttaattaaaaataaagagcatttaactgaagaaggtttaaatcaaattttaactttaaaagcttcattaaatttaggtttatcagaagaattaaaagaggctttccctaacactataccagccgaaaaacttttagttacaggtcaagaaataccagattcaaactgagtagctggttttacagctggtgaaggatctttttatattagaatagctaaaaattctacactaaaaacaggataccaagttcaatctgtctttcaaattactcaagatactcgagatatagaattaatgaaaaatttaatatcttatttaaattgtggaaatattagaataagaaagtataaaggttctgaaggtatacatgatacttgtgtagatttagttgtaactaatctgaatgatataaaagaaaaaattatacctttttttaataaaaatcatataataggagttaaattacaagattatagagattgatgtaaagtagttactttaattgataataaagaacatttaacttcagaaggtttagaaaaaattcaaaaaataaaagaaggtatgaataggggaagatctctttagatcaagtataggagcatttgccataaaaggctgaataagcggaaaataataaaatattagggtttaattacaattaataaaaaaaatgaagaaatagtctgaaccat

SEQ ID NO: 3 - protein, 712 aa, Leptographium truncatum
MQKDTKFLNKSNIFIKNINNKYKLIPFNIKINFVGENKNFPSDFKEWTNNIYYFNSNYIKNFPVYDLNLNKLLKGYFDLYFNRESIQSKFKFFRKRRLSLNKIFVSKPEIKHTSSKTTITVYVYNRERIVLLKKLIKLRKSLFKINNFFYKCKSISGDLYGKYFINVLYKELVYIRRCKLKLNLNELKFKDQFLHKLSLLISKLYNKKVEFNIINLKSIVYNSSIFTEIMGKKLRNKNTSLLKTMKFILSKGIILEENNKKERSRLIKSVNFSLLENKYKNLNINSFVKDIDLNETIKDLYNIESKDNKDIVFDSIKYKNIGGIRLEAKGRLTKRYRADRALFKVNWKGGLKNTDSSYKGLSSVNFRGNLKSNVEYSMGISKRRIGAFAVKGWISGKSYSTLANFPVQARNDNISPWTITGFADAESSFMLTVSKDSKRNTGWSVRPRFRIGLHNKDVTILKSIREYLGAGIITSDIDARIRFESLKELEVVINHFDKYPLITQKRADYLLFKKAFYLIKNKEHLTEEGLNQILTLKASLNLGLSEELKEAFPNTIPAERLLVTGQEIPDSNWVAGFTAGEGSFYIRIAKNSTLKTGYQVQSVFQITQDTRDIELMKNLISYLNCGNIRIRKYKGSEGIHDTCVDLVVTNLNDIKEKIIPFFNKNHIIGVKLQDYRDWCKVVTLIDNKEHLTSEGLEKIQKIKEGMNRGRSL

SEQ ID NO: 4 - DNA, 2614 bp, Leptographium truncatum
ataattgatatacatatkgggttaatttcaagaattacttatattgtaataagtaaatttgaagcgaagtttattttaacctttcgtccccgaccatacgacccactatttaggggtgtttaatatttagggcgcgagctcttaagagctccgcgcttaaaaactaaaaagactaggtcgagggattaaaaaagaaataaaattgttcaacgactaagagtgagttatataacaataatctcttattaatacccaacattttttttattatkgwaagttagtttttataactataaaagaataaatgcaaaaagatacaaaatttttaaataaaagtaatatatttattaaaaatattaataataaatataaattaataccgttcaatattaaaattaactttgtaggtgaaaataaaaattttccttctgactttaaagaatgaactaataatatctattattttaattctaattatattaaaaattttcctgtatatgatttaaatttaaataaattattaaaaggttattttgatttatattttaatcgtgaaagtattcaatctaaatttaaattttttagaaaaagacgtttatctttaaacaaaatatttgtaagtaaacctgaaataaaacatactagctctaaaactacaattacagtgtatgtttataatagagaaagaattgtcttattaaaaaaattaattaaactaagaaagtcattatttaaaataaataatttcttttataaatgtaaaagtatttctggggatctgtatggtaaatattttataaatgttttatacaaagaattagtttatataagaagatgtaaattaaaacttaatcttaatgaattaaaatttaaagatcaatttttacataaattaagtctattaataagtaaattatataataaaaaagtagagtttaacattattaatttaaaatctattgtttataattctagtatttttacagaaataatgggtaaaaaattaagaaataaaaatacaagtcttttaaaaacaatgaaatttattttaagtaaaggtattattttagaggaaaataataaaaaagaaagaagtagattaattaaaagtgtaaatttcagtcttttagagaataaatataaaaatcttaatattaattctttcgttaaagatatagatctaaatgaaacaataaaagatttatataatattgaaagtaaagataataaagatattgtatttgattctattaaatacaaaaacataggaggtataagattagaagctaaaggtagattaactaagcgttatagagcagatagagcattatttaaagttaattgaaaaggaggattaaaaaatacagattcatcttataaaggattgtcatccgtaaactttagaggaaatcttaagtctaatgtggaatattcaatgggtatatctaaacgtagaataggagcttttgctgttaaaggttgaatatcaggtaaatcttatagtacattggctaattttccggtgcaagctagaaatgacaatattagtccttgaactattacaggatttgcagacgctgaaagttcttttatgcttactgttagtaaagatagtaaacgtaatacaggatgatcagttagaccacgttttcgtataggtttacataataaagatgtaactatattaaaaagtattcgtgaatatttaggtgcaggaataattacttctgatatagatgctagaataagatttgaatctttaaaagaattggaggtagttataaatcattttgataaatatccattaataactcaaaaacgtgcggactatcttctttttaaaaaagcattttatttaattaaaaataaagagcatttaactgaagaaggtttaaatcaaattttaactttaaaagcttcattaaatttaggtttatcagaagaattaaaagaggctttccctaacactataccagccgaaagacttttagttacaggtcaagaaataccagattcaaactgagtagctggttttacagctggtgaaggatctttttatattagaatagctaaaaattctacactaaaaacaggataccaagttcaatctgtctttcaaattactcaagatactcgagatatagaattaatgaaaaatttaatatcttatttaaattgtggaaatattagaataagaaagtataaaggttctgaaggtatacatgatacttgtgtagatttagttgtaactaatctgaatgatataaaagaaaaaattatacctttttttaataaaaatcatataataggagttaaattacaagattatagagattgatgtaaagtagttactttaattgataataaagaacatttaacttcagaaggtttagaaaaaattcaaaaaataaaagaaggtatgaataggggaagatctctttagatcaagtataggagcatttgccataaaaggctgaataagcggaaaataataaaatattagggtttaattacaattaataaaaaaaatgaagaaatagtctgaaccattttgtgaaaaatggaaataaaaatttttatgataacaagttgaacagctaatttgcgcaagagg

SEQ ID NO: 5 - protein, 725 aa, Sporothrix sp.
MENNIKLKNSLNKNKSNIFNKYINNKYKLVPFKTMINYVNEPRYIPSEFKEWNNSIYYFNFTNIKNLPVYDINLNKLLKSYFDLYFFSASHPSSLVRERRANDKKNNFIPIIKKKNLFSLNKLFIRKADLKHTSPKKIITIYIFNRERIILIKNLIYLYSLHFKTKSYLEENKDIFFYQSFKEKLNNKYEIFNKIKLNFNLNNLKFKDIMLYKLSKLLSKFYKKKVEFNIINLNSYKYNSDIVTDILKKKVVKPNSKLLKIMKFIAKKSLKASIGKTADKYKDKTRISKSINYDLIPNKYKNLNISVIIKNINFNETIKNIYNIKNNTNENIIYNSIKYKLVGGVRLEIKGRLTRRYRADRSKFYAETVGTLQNIDSSFKGLSSKLYRGKFNSNMQYSIDVYKRHVGSYAVKGWISGRSYSTSAYIPKSESINPWVITGFADAEGSFLLRIRNNNKSSVGYYTELGFQITLHNKDKSILENIQSTWKIGVIANSGDNAVSLKVTRFEDLKVIINHFDKYPLITQKLGDYLLFKQAFSVMENKKHLKIEGIKELVEIKAKLNWGLTDELKKAFPETISKERSLINKDIPNFEWLAGFTSGEGCFFVNLIKSKSKLGVQVQLVFSITQHIKDKYLMNSLISYLGCGYIKEKNKSEFSWLDFVVTKFSDINDKIIPVFQKNTLIGIKTKDFEDWCKVAKLIEEKKHLTESGLDEIKKIKLNMNKGRVF

SEQ ID NO: 6 - DNA, 2357 bp, Sporothrix sp.
aataattattaaataataattaattttattaataccccacatttctttttattaaataaataagtttaaaatttaaatttaaaatttggaaaataatataaaattaaaaaattcattaaataaaaataaaagtaatatatttaataaatatattaacaataaatataaattagttccgtttaaaactatgattaattatgtaaatgaacctagatatatcccttctgaatttaaagaatgaaataatagtatctattattttaattttactaatattaaaaatttacctgtgtacgatataaatttaaataaattattaaaaagttattttgatttatattttttttcggcttcgcatcccagctctcttgttagagagcgtagggctaatgataaaaaaaataactttatccctattattaaaaaaaaaaaccttttttctttaaataaactatttattagaaaagcagatttaaaacatactagtcctaaaaaaataattactatttatatttttaatagagaaagaataattttaataaaaaatcttatttatttatattctttacattttaaaacaaaatcatatttagaagaaaataaagatatttttttttatcagtcttttaaagaaaaattaaataataaatatgaaatttttaataaaattaaattaaattttaatcttaataatttaaaatttaaagatataatgctatataaattaagcaaattattaagtaaattttataaaaaaaaagtagaatttaatattattaatttaaattcatataaatataattctgatattgttacagatatattaaaaaaaaaagtagtgaaacctaattctaaattattaaaaataatgaaatttattgcaaaaaaaagtttaaaagcttcaatagggaagactgctgataaatataaagataaaactagaatatctaaatctataaattatgatttaatacctaataaatataaaaatttaaatataagtgttataattaaaaatattaattttaatgaaactattaaaaatatatataatattaaaaataatactaatgaaaatattatatacaattctattaagtataaacttgtaggaggtgtaagattagaaataaaaggtagattaacaagacgttatagagcggatagatcaaaattttatgcggaaacagtaggaactttacaaaatatagattcatcatttaaagggttatcatctaaactttatagaggtaaatttaattctaatatgcaatattcgatagatgtgtataaacgtcatgtaggatcatatgctgtaaaaggttggatttcaggtagatcttacagtacttctgcgtacataccaaaaagtgaaagtataaacccttgagttattactggatttgcagatgctgaaggaagttttttattaagaataagaaataataataaaagttctgtaggttattatacagaattaggatttcaaattactctacataataaagacaaatctatcttagaaaatattcaatctacttgaaaaataggagttatagctaatagcggtgataatgctgtaagtttaaaagtaacacggtttgaagatttaaaagtaataataaatcattttgataaatatcctttgataactcaaaaattaggtgactatcttttatttaaacaagcatttagtgttatggagaacaaaaaacatttaaaaattgaaggtataaaagaattagttgaaattaaggctaaattaaattgaggtcttactgatgaattaaaaaaggctttccctgaaactatttcgaaagaaagatctttaataaataaagatatacctaattttgaatgattagctggctttacttctggtgaaggttgtttttttgttaatttaataaaatctaaatctaaattaggagttcaagtacaattggtattttctattactcaacatattaaggataaatatttaatgaatagtttaatttcatatcttggatgtggttatattaaggaaaaaaataaatctgagttttcatgattagattttgttgttacaaaattttcagatattaatgataaaattatcccagtttttcaaaaaaatactttaataggtataaaaacgaaagactttgaagattgatgtaaagtcgcaaaattaattgaagaaaaaaaacatttaactgaatctggtttagatgaaataaaaaaaataaaattaaacatgaataaaggaagagttttttaagttaactatattttaaataatataaaaataattgtataattttggttgaataagtggaaaataataatatttaataaaaaaaaagaaatgaact

SEQ ID NO: 7 - protein, 684 aa, Ophiostoma ulmi
MENNIKLENSCCALNKSNIFNKYINNKYKLVPFKTLVNYVNEPRYIPSEFKEWNNSIYYFNFNNIKNLPVYDINLNKLLKSYFDLYFISKNKNNKFISIIKKKQRYSLNKIFISKADLKHTSSKIIITIYIFNRERIILIKNLIFLYSLHFKTKSYLEKNKNLFFFESFKKKLNNKYEIFNKLKLNFNLNNLKFKDIMLYKLSKLLSKFYNKKVEFNIINLNSYKYNSDILTDIFKKKVVNPNSKLIKIMKFIGKKSLRASIGKTGDNYMDKTRISKSINYDLIPNKYKNLNISLIIENINFNETIKNIYNISNDTNENIIYNSIKYKLVGGVRLAIKGRLTKRYRADRSKLYSKTVGNLQNIDSSFKGLSSKLYRNKLNSNMQYTLDVYKRHVGAYAVKGWISGRSYSTSAYIPRKQSIDPWLLTGFTDAEGTFLLRIRNNNKSSVGYSTELGFQITLHNKDKSILENIQSTWNVGVIANSGDNAVSLKVTRFNDLKVVINHFEQYPLITQKLGDYMLFKQAFSVMENKEHLKIEGIKELVRIKAKLNWGLTDELKMAFPEIIFEERPLINKNIPNFKWLAGFTSGEGCFFINLIKSKSKLGVQVQLVFSITQHIKDRDLMNSLITYLGCGYIKEKNKSEFSWLDFVVTKFSDINNKIIPVFQENNLVGIKLKDFEDWCKVAK

SEQ ID NO: 8 - DNA, 2546 bp, Ophiostoma ulmi
ggaaaagctacgctagggatgttagtccttcaatattttttaataattgataataatattgggttaatttcaagaattacttatatttataagtaaatttgaagcgaaatttattttttttaaataaaactgttcaacgactaaaaataattattatttaataattatttttattaatacccaacatttctttttattaaataaataagtttaaaatttaaatttaaaatttggaaaataatataaaattagaaaattcgtgctgcgccttaaataaaagtaatatatttaataaatatattaataataaatataaattagttccgtttaaaactttggttaattatgtaaatgaacctagatatataccttctgaatttaaagaatgaaataatagtatttattattttaattttaataatattaaaaatttacctgtatatgatataaatttaaataaattattaaaaagttattttgatttatattttatttctaaaaataaaaataataaatttatttctattataaaaaaaaaacaacgttattctttaaataaaatatttattagtaaagcagatttaaaacatactagttctaaaataataattactatttatatttttaatagagaaagaataattttaataaaaaatcttatttttttatattctttacattttaaaacaaaatcatatttagaaaaaaataaaaatttatttttttttgagtcttttaaaaaaaaattaaataataaatatgaaatttttaataaattgaaattaaattttaatcttaataatttaaaatttaaagatataatgttatataaattaagtaaattattaagtaaattttataataaaaaagtagaatttaatattattaatttaaattcatataaatataattctgatattcttacagatatatttaaaaaaaaagtagttaatcctaattctaaattaataaaaataatgaaatttattggaaaaaaaagtttaagagcttcaatagggaagactggtgataattatatggataaaactagaatatctaaatctataaattatgatttaatacctaataaatataaaaatttaaatataagtcttataattgaaaatattaattttaatgaaactataaaaaatatatataatattagtaatgatactaatgaaaatattatatataattctattaagtataagcttgtaggaggtgtaagattagctataaaaggtagattaactaaacgttatagagcggatagatcaaaactgtattcaaaaacagtaggaaatttacaaaatatagattcatcatttaaaggattatcatctaaactttatagaaataaattaaattctaatatgcaatatacactagatgtatataaacgtcatgtgggagcatatgcagtaaaaggttgaatttcaggtagatcttacagtacttctgcgtatataccgaggaaacagagtatagacccttgattacttactggatttactgatgctgaaggaacttttttattaaggataagaaataataataaaagttctgtgggttattctacagaattaggatttcaaattactttacacaataaagacaaatctattttagaaaatattcaatctacttgaaatgtaggagttatagctaatagcggtgataatgccgtaagtttaaaagtaactcggtttaatgatttaaaagtagtaataaatcattttgagcaatatcctttgataactcaaaaattaggtgactatatgttatttaaacaagcatttagtgttatggagaacaaagaacacttaaaaattgaaggtataaaagaattagttaggattaaggccaaattaaattgaggtcttactgatgaactaaaaatggctttccctgaaattatttttgaggaaagacctttaataaataaaaatatacctaattttaagtgattagctggttttacatctggtgaaggttgtttttttattaatttgataaaatctaaatctaaactaggtgttcaagtacaattggtattttctattactcagcatattaaagatagagatttaatgaatagtttaataacatatctaggatgtggttatattaaggagaaaaataaatctgagttttcatgattagattttgttgttacaaaattttcagatattaataataaaattattccagtttttcaagaaaataatctagtaggtataaaattgaaagattttgaagattggtgtaaagttgcaaaataattgaagaaaaaaaacatttaactgagtctggtttagatgaaattaaaaaaataaaattaaacatgaataaaggaagagttttttaaattaactatatttttactaatataaaaataattgaaacattttggttgaataagtggaaaataataatatttaataaaaaaaaaaaagaaatgaagaaatagtctgaaccattttgtgaaaaatggaaataaaaatttatgataacaagttgaacaggctaatttgcgcaagaag

SEQ ID NO: 9 - protein, 481 aa, Grosmannia piceiperda
MQKDTKFLNKSNIFIKNINNKYKLIPLNIKINFVGENKYFPSDFKEWTNNIYYFNSNYIKNFPVWWFGKSSFIWDKLSNSGNVLKSLVPSNTRKVICGWSNYSCMVISQWMNESEMDNRGSKSVLFNNLTVKEQRVKGSYCINGIQLRCTLVGFERNYQVKIPSNQIINKSIRYISNSATVTPLIDPWFITGFADAESSFVVSIKRNKKIKCGWNVVTRFQIALSQKDLALLERIKSYFKDAGNIYIKSDKVSVDWHVTSVKDLKIILDHFDKYPLKTEKLADYILFKEVFNIILTKQHLTVEGIQKIVAIRASINKGLYGELKAAFPNIIPVQRPKIDDRFIIDIQPWWVAGFTEGEGCFSVVVTNSPSTKSGFSASLIFQITQHSRDIVLMQNIIKFLGCGRIHKRSKEEAVDILVTKFSDLTEKVIPFFESIPLQGLKLKNFTDFSKAADIIKVKGHLTPKGLDKILQIKLGMNTRRI

SEQ ID NO: 10 - DNA, 2928 bp, Grosmannia piceiperda
gtatacatattgggttaatttcaagaattacttatattgtaataagtaaatttgaagcgaagtttattttaacctttcgtccccgaccatacgacccactatgtagggatgtttaatttttagggtgcgagctcttaatttttaaaaactaaaaagactaggtcgagggattaaaaaagaaataaaattgttcaacgactaagagtgagttatataacaataatctcttattaatacccaacattttttttattattgtaagttagtttttataactataaaagaataaatgcaaaaagatacaaaatttttaaataaaagtaatatatttattaaaaatattaataataaatataaattaataccgttaaatattaaaattaactttgtaggtgaaaataaatattttccttctgactttaaagaatgaactaataatatctattattttaattctaattatattaaaaattttcctgtttgatgatttgggaagtcatcttttatatgggataaactatcaaattccgggaacgtcctaaagtcattggtaccaagcaatacccgaaaggttatttgtggatgaagtaattactcatgtatggtaataagtcaatgaatgaacgaaagtgaaatggataatcgcggatctaaatcagtattgtttaataaccttactgtaaaagagcaacgagtaaaaggtagttattgtataaatggtatacaattaagatgtactctagtgggtttcgaaagaaattatcaagtcaaaatcccatctaatcaaataataaataaatcaataagatatatctcaaattcagctacggttactcctttaatagatccttgatttataacaggatttgctgatgctgaaagttcttttgtagtttcaattaagagaaataaaaaaatcaaatgtggttgaaatgtagtaactagattccaaatagctttatctcaaaaggatttagctttattagaacgtattaaaagctatttcaaagatgccgggaatatttatataaaaagcgataaagtgtcggttgattgacacgtaacttcggtaaaagatttaaagataatcttagaccattttgataaataccctttaaaaactgagaaattagctgattacatactgtttaaagaagtgtttaatataattttaactaaacaacatctaacagttgaaggtatacagaaaattgtagcaattagagcatcaataaataaaggtttatatggtgaattgaaagctgcttttccgaatattattcctgttcaaagacctaaaattgatgatagatttattatcgacatccaaccttgatgagtagcaggttttactgaaggggaaggatgttttagtgttgtggttacgaattcgccttctactaaaagtggattttcggcaagtttgatttttcaaataactcaacattcaagggatattgtattaatgcaaaatataataaaatttttaggttgtggtagaatacataagagatctaaggaggaagctgtcgatattttagtaactaaattttcagatttgactgagaaagttatcccgttttttgaaagtatacctttgcaaggtttaaaacttaaaaactttacggatttctctaaagcggctgatataataaaagttaaaggacacttaactccgaaaggtttagataaaatattacaaataaaattaggaatgaacacaagaagaatttaatttacagatcacctccgatgatctgcaaaagccggagggcgatcttaaataagagctccgcgccgggtgttttaaaagtttatttattatttgtatgataaatttggtatccgtggtatatgacttaaatttaaataaattattaaaaggttattttgatttatattttaatcgtgaaagtattcaatctaaatttaaattttttagaaaaagacgtttatctttaaacaaaatatttgtaagtaaacctgaaataaaacatactagctctaaaactacaattacagtgtatgtttataatagagaaagaattgtcttattaaaaaaattaattaaactaagaaagtcattatttaaaataaataatttcttttataaatgtaaaagtatttctggggatctgtatgggaaatattttataaatgttttatacaaagaattagtttatataagaagatgtaaattaaaacttaatcttaatgaattaaaatttaaagatcaatttttacataaattaagtctatgaataagtaaattatataataaaaaagtagagtttaacattattaatttaaaatctattgtttataattctagtatttttacagaaataatgggtaaaaaattaagaaataaaaatacaagtcttttaaaaacaatgaaatttattttaagtaaaggtattattttagaggaaaataataaaaaagaaagaagtagattaattaaaagtgtaaatttcagtcttttagagaataaatataaaaatcttaatattaattctttcgttaaagatatagatctaaatgaaacaataaaagatttatataatcttgaaagtaaagataataaagatattgtatttgattctattaaatacaaaaacataggaggtataagattagaagctaaaggtagattaactaagcgttatagagcagatagagcattatttaaagttaattgaaaaggaggattaaaaaatacagattcatcttataaaggattgtcatcagtaaactttagaggaaatcttaagtctaatgtggaatattcaatgggtatatctaaacgtagaataggagcatttgccataaaaggctgaataagcggaaaataataaaatattagggtttaattacaattaataaaaaaaatgaagaaatagtctgaaccatttggtgaaaaaggca

SEQ ID NO: 11 - protein, 727 aa, Grosmannia penicillata
MNKMQKYTKFLSNIFVKNINNKYKSIPLNTRINFVGETRYFPSDFKEWTNSVYYFNSNNIKNFPIYDLNVSKLLKGYFDLYFDRENIKLNYKSYEKKDFTAQALNKIFVSKPEIKHTNSKAIITIYVYNRERVILLNKLNQLNIGILGLNKFFYLCKKISGDLYSKYIKQVLYKEFLFLRRSKLKLNLNGLKFQDKFLFKLSKLISKFYKKKVEFNIINLKSINLNPNIFTEIMAKKFMNRNASIMQIMKFILDKSIILNEDGGIVSLASSGVTSSGSGLEKSRKIKNVNLNLIENKYKNLNINSIVTDGDINDNIKEFYTKNSEDTIFDSKYKNLGGIRLEAKGRLTRRYRADRAVSKVNIKGGLKNIDSSYKGLSSINYIGKINSSMEYSMDISKRRVGAFAIKGWISGRSYSTTANPTRNESINPWVLTGFADAEGSFILRIRNNNKSSAGYSTELGFQITLHKKDISILENIQSTWKVGVIANSGDNAVSLKVTRFEDLRVVLNHFEKYPLITQKLGDYLLFKQAFSVMENKEHLKIEGIKRLVGIKANLNWGLTDELKEAFVASGGENIFVASGGERSLINKNIPNSGWLAGFTSGEGCFFVSLIKSKSKLGVQVQLVFSITQHARDRALMDNLVTYLGCGYIKEKKKSEFSWLEFVVTKFSDIKDKIIPVFQVNNIIGVKLEDFEDWCKVAKLIEEKKHLTESGLEEIRNIKLNMNKGRVL

SEQ ID NO: 12 - DNA, 2522 bp, Grosmannia penicillata
gggttaatttcaagaattacttatactataagtaaatttgaagcgaagtttatttatatttaataaataataatgttcaacgactaagggtgagttaataacaataatcccttattaatacccaacactttttttttgtaatttagtttaataatataaaatcaataaaatgcaaaaatataccaaatttttaagtaatatatttgtcaaaaatattaataataaatataaatctataccattgaatactagaattaattttgttggtgaaactagatatttcccttctgattttaaagaatgaactaatagtgtttattactttaattctaataatattaaaaattttcccatttatgacttaaatgtaagtaaattattaaaaggttattttgatttatattttgatcgtgaaaatataaaattgaattataaatcttatgaaaaaaaagattttacggcgcaagctctaaataaaatctttgtaagtaaacctgaaataaaacatactaattctaaagccataataactatttatgtttataatagagaaagagttattttgttaaataaattaaatcaattaaatataggaattttaggactaaataaatttttttatttatgtaaaaaaatctctggggatttatatagtaaatatattaaacaagttttatataaagaattcttatttttaagaagatctaaattaaaacttaatcttaatggattaaaattccaagataaattcttatttaaattaagtaaattaataagtaaattttataaaaaaaaagtcgaatttaatattattaatttaaaatctataaatttgaatcctaatatatttactgaaataatggcaaaaaaatttatgaatagaaatgcatctattatgcaaataatgaaatttatcttagataaaagtattattttaaatgaggatggcggcatagtttcactagcctcatccggtgtaacctcctccggatcaggattagaaaaaagtagaaaaattaaaaatgtaaatttaaatttaatagaaaataaatataaaaatctgaatattaactctatagttactgatggagatataaatgataatataaaagaattttatactaaaaatagtgaagatactatttttgattctaaatataaaaatttaggaggtataagattagaagctaaaggaagattgactagacgttatagagcggatagagcagtatctaaagttaatataaaaggaggattgaaaaatatagattcatcttacaaaggtttatcttctattaattatataggaaaaattaattcaagtatggaatattcaatggatatatcaaaacgtcgtgtaggtgcatttgctataaagggttgaatttcaggtagatcttacagtacaactgccaatcctacaagaaatgaaagtataaatccttgagttcttactgggtttgcagatgctgaaggtagttttatactaagaataagaaataataataaaagttctgcaggttattctacagaattaggatttcaaattactttgcataaaaaagacatatctatcctagaaaatattcaatctacttgaaaagtaggagttatagctaatagcggtgataacgccgtaagtttaaaagtaacacggtttgaggatttaagagtagtattaaatcattttgagaaatatcctttgataactcaaaaattaggtgattatctattatttaaacaagcctttagtgttatggaaaacaaagaacatttaaaaattgaaggtataaaaagattagttggaattaaggccaatttaaattgaggtcttactgatgaactaaaggaggcttttgtcgcctccggcggtgaaaatatatttgtcgcctccggcggtgaaagatctctaataaataaaaatatacctaattctggatgattagctggctttacttctggtgagggttgtttttttgttagtttaataaaatctaaatctaaattaggggttcaggtacaattggtattttctattactcaacacgcgagagatagagcattgatggataatttagtaacatatcttggatgtggatatattaaggaaaaaaagaaatctgagttttcatgattagaatttgttgttacaaagttttcagacattaaggataaaattattccagtttttcaagtaaataatataataggtgtaaaattggaagactttgaagattgatgtaaagtcgctaaattaattgaagaaaaaaaacatttaactgaatcaggcttggaagaaatacgtaatataaaattaaacatgaataaaggaagagttctttaagttaactacatactaagtgcagtatataatttcaatatttaaactctagtatattttaatactacgttagcaagctcccatacttcgtatgagaataagcggaaaataattttttttaggttttaattacaataaataaaaaaaagtgaagaaatagtctgaaccattttgtgaaaaa

SEQ ID NO: 13 - protein, 715 aa, Ophiostoma novo-ulmi subsp. americana
MENNIKLENSCCALNKNKSNIFNKYINNKYKLVPFKTLVNYVNEPRYIPSEFKEWNNSIYYFNFNNIKNLPVYDINLNKLLKSYFDLYFISKNKNNKFISIIKKKQRYSLNKIFISKADLKHTSSKIIITIYIFNRERIILIKNLIFLYSLHFKTKSYLEKNKNLFFFESLKKKLNNKYEIFNKLKLNFNLNNLKFKDIMLYKLSKLLSKFYNKKVEFNIINLNSYKYNSDILTDIFKKKVVNPNSKLIKIMKFIGKKSLRASIGKTGDNYMDKTRISKSINYDLIPNKYKNLNISLIIENINFNETIKNIYNISNDTNENIIYNSIKYKLVVGVRLAIKGRLTKRYRADRSKLYSKTVGNLQNIDSSFKGLSSKLYRNKLNSNMQYTLDVYKRHVGAYAVKGWISGRSYSTSAYMSRRESINPWILTGFADAEGSFLLRIRNNNKSSVGYSTELGFQITLHNKDKSILENIQSTWKVGVIANSGDNAVSLKVTRFEDLKVIIDHFEKYPLITQKLGDYMLFKQAFCVMENKEHLKINGIKELVRIKAKLNWGLTDELKKAFPEIISKERSLINKNIPNFKWLAGFTSGEGCFFVNLIKSKSKLGVQVQLVFSITQHIKDKNLMNSLITYLGCGYIKEKNKSEFSWLDFVVTKFSDINDKIIPVFQENTLIGVKLEDFEDWCKVAKLIEEKKHLTESGLDEIKKIKLNMNKGRVF

SEQ ID NO: 14 - DNA, 2563 bp, Ophiostoma novo-ulmi subsp. americanum
tggattattggaaaagctacgctagggatgttagtccttcaatattttttaataattgataataatattgggttaatttcaagaattacttatatttataagtaaatttgaagcgaaatttattttttttaaataaaactgttcaacgactaaaaataattattatttaataattatttttattaatacccaacatttctttttattaaataaataagtttaaaatttaaatttaaaatttggaaaataatataaaattagaaaattcgtgctgcgccttaaataaaaataaaagtaatatatttaataaatatattaataataaatataaattagttccgtttaaaactttggttaattatgtaaatgaacctagatatataccttctgaatttaaagaatgaaataatagtatttattattttaattttaataatattaaaaatttacctgtatatgatataaatttaaataaattattaaaaagttattttgatttatattttatttctaaaaataaaaataataaatttatttctattataaaaaaaaaacaacgttattctttaaataaaatatttattagtaaagcagatttaaaacatactagttctaaaataataattactatttatatttttaatagagaaagaataattttaataaaaaatcttatttttttatattctttacattttaaaacaaaatcatatttagaaaaaaataaaaatttatttttttttgagtctttaaaaaaaaaattaaataataaatatgaaatttttaataaattgaaattaaattttaatcttaataatttaaaatttaaagatataatgttatataaattaagtaaattattaagtaaattttataataaaaaagtagaatttaatattattaatttaaattcatataaatataattctgatattcttacagatatatttaaaaaaaaagtagttaatcctaattctaaattaataaaaataatgaaatttattggaaaaaaaagtttaagagcttcaatagggaagactggtgataattatatggataaaactagaatatctaaatctataaattatgatttaatacctaataaatataaaaatttaaatataagtcttataattgaaaatattaattttaatgaaactataaaaaatatatataatattagtaatgatactaatgaaaatattatatataattctattaagtataaacttgtagtaggtgtaagattagctataaaaggtagattaactaaacgttatagagcggatagatcaaaactgtattcaaaaacagtaggaaatttacaaaatatagattcatcatttaaaggattatcatctaaactttatagaaataaattaaattctaatatgcaatatacactagatgtatataaacgtcatgtaggagcatatgcagtaaaaggttgaatttcaggtagatcttacagtacttctgcatatatgtcgaggagggaaagtataaatccttgaattcttaccggatttgcagatgctgaaggaagttttttattaagaataagaaataataataaaagttctgtaggttattctacagaattaggatttcaaattactttacataataaagacaaatctattttagaaaatattcaatctacttgaaaagtaggagttatagctaatagcggtgataatgccgtaagtttaaaagtaacacggtttgaagatttaaaagtaataatagatcattttgagaaatatcctttgataactcaaaaattaggtgattatatgttatttaaacaagcattttgtgttatggagaacaaagaacatttaaaaattaatggtataaaagaattagttaggattaaagccaaattaaattgaggtcttactgatgaactaaaaaaggctttccctgaaattatttctaaagaaagatctttaataaataaaaatatacctaattttaaatgattagctggttttacttctggtgaaggttgtttttttgttaatttaataaaatctaaatctaaattaggtgttcaagtacaattggtattttctattactcaacatattaaagataaaaatttaatgaatagtttaataacatatctcggatgtggttatattaaggaaaaaaataaatctgaattttcatgattagattttgttgttacaaaattttcagatattaatgataaaattattccagtttttcaagaaaatactttaataggtgtaaaattggaagattttgaagattgatgtaaagtagcaaaattaattgaagaaaaaaaacatttaactgaatctggtttagatgaaattaaaaaaataaaattaaacatgaataaaggaagagttttttaaattaactatatttttaatagtataaaaataattatgaaattttggcaagaataagtggaaaataataatatttaataaaaaaaaaagaaatgaagaaatagtctgaaccattttgtgaaaaatggaaataaaaatttatgatatgataacaagttgaacaggctaatttgcgcaag

SEQ ID NO: 15 - protein, 712 aa, Leptographium pityophilum
  misc_feature (500)..(500): Xaa can be any naturally occurring amino acid
MQKDTKFLNKSNIFIKNINNKYKLIPFNIKINFVGENKYFPSDFKEWTNNIYYFNSNYIKNFPVYDLNLNKLLKGYFDLYFNRESIQSKFKFFRKRRLSLNKIFVSKPEIKHTSSKTTITVYVYNRERIVLLKKLIKLRKSLFKINNFFYKCKSISGDLYGKYFINVLYKELVYIRRCKLKLNLNELKFKDQFLHKLSLLISKLYNKKVEFNIINLKSIVYNSSIFTEIMGKKLRNKNTSLLKTMKFILSKGIILEENNKKERSRLIKSVNFSLLENKYKNLNINSFVKDIDLNETIKDLYNLESKDNKDILFGSIKYKNIGGIRLEAKGRLTKRYRADRALFKVNWKGGLKNTDSSYKGLSSVNFRGNLKSNVEYSMGISKRRIGAFAVKGWISGKSYSTLANFPVQARNDNISPWTITGFADAESSFMLTVSKDSKRNTGWSVRPRFRIGLHNKDVTILKSIREYLGAGIITSDKDARIRFESLKELEVVINHFDKYXLITQKRADYLLFKKAFYLIKNXEHLTEEGLNQILTLKASLNLGLSEELKEAFPNTIPAERLLVTGQEIPDSNWVAGFTAGEGSFYIRIAKNSTLKTGYQVQSVFQITQDTRDIELMKNLISYLNCGNIRIRKYKGSEGIHDTCVDLVVTNLNDIKEKIIPFFNKNHIIGVKLQDYRDWCKVVTLIDNKEHLTSEGLEKIQKIKEGMNRGRSL

SEQ ID NO: 16 - DNA, 2570 bp, Leptographium pityophilum
  misc_feature (1800)..(1800): n is a, c, g, or t
ttttttttaataattgatatacatattgggttaatttcaagaattacttatattgtaatgtaataagtaaatttgaagcgaagtttattttaaccttttgtccccgaccttacgacccattatgtaggggtgtttaatttttagtttttaaaaattaaaaactaaaaagactaggtcgagggattaaaaaagaaataaaattgttcaacgactaagagtgagttatataacaataatctcttattaatacccaacattttttttattattgtaagttagtttttataactataaaagaataaatgcaaaaagatacaaaatttttaaataaaagtaatatatttattaaaaatattaataataaatataaattaataccgttcaatattaaaattaactttgtaggtgaaaataaatattttccttctgactttaaagaatgaactaataatatctattattttaattctaattatattaaaaattttcctgtatatgacttaaatttaaataaattattaaaaggttattttgatttatattttaatcgtgaaagtattcaatctaaatttaaattttttagaaaaagacgtttatctttaaacaaaatatttgtaagtaaacctgaaataaaacatactagctctaaaactacaattacagtgtatgtatataatagagaaagaattgttttattaaaaaaattaattaaactaagaaagtccttatttaaaataaataatttcttttataaatgtaaaagtatttctggggatctgtatgggaaatattttataaatgttttatacaaagaattagtatatataagaagatgtaaattaaaacttaatcttaatgaattaaaatttaaagatcaatttttacataaattaagtctattaataagtaaattatataataaaaaagtagagtttaacattattaatttaaaatctattgtttataattctagtatttttacagaaataatgggtaaaaaattaagaaataaaaatacaagtcttttaaaaacaatgaaatttattttaagtaaaggtattattttagaagaaaataataaaaaagaaagaagtagattaattaaaagtgtaaatttcagtcttttagagaataaatataaaaatcttaatattaattctttcgttaaagatatagatctaaatgaaacaataaaagatttatataatcttgaaagtaaagataataaagatattctatttggttctattaaatacaaaaacataggaggtataagattagaagctaaaggtagattgactaaacgttatagagcagatagagcattatttaaagttaattgaaaaggaggattaaaaaatacagattcatcttataaagggttatcatcagtaaactttagaggaaatcttaagtctaatgtagaatattcaatgggtatatctaaacgtagaataggagcttttgctgttaaaggttgaatatcaggtaaatcttatagtacattggctaattttccggtgcaagctagaaatgacaatattagtccttgaactattacaggatttgcagacgctgaaagttcttttatgcttactgttagtaaagatagtaaacgtaatacaggatgatcagttagaccacgttttcgtataggtttacataataaagatgtaactatattaaaaagtattcgtgaatatttaggtgcaggaataattacttctgataaagatgctagaataagatttgaatctttaaaagaattggaggtagttataaatcattttgataaatatncattaataactcaaaaacgtgcggactatcttctttttaaaaaagcattttatttaattaaaaatnaagagcatttaactgaagaaggtttaaatcaaattttaactttaaaagcttcattaaatttaggtttatcagaagaattaaaagaggctttccctaacactataccagccgaaagacttttagttacaggtcaagaaataccagattcaaactgagtagctggttttacagctggtgaaggatctttttatattagaatagctaaaaattctacactaaaaacaggataccaagttcaatctgtctttcaaattactcaagatactcgagatatagaattaatgaaaaatttaatatcttatttaaattgtggaaatattagaataagaaagtataaaggttctgaaggtatacatgatacttgtgtagatttagttgtaactaatctgaatgatataaaagaaaaaattatacctttttttaataaaaatcatataataggagttaaattacaagattatagagattgatgtaaagtagttactttaattgataataaagaacatttaacttcagaaggtttagaaaaaattcaaaaaataaaagaaggtatgaataggggaagatctctttagatcaagtataggagcatttgccataaaaggttgaataagcggaaaataataaaatcttagggtttaattacaattaataaaaaaaatgaagaaatagtctgaaccattctgtgaagaaaggaaataaaa

SEQ ID NO: 17 - DNA, 18 bp, Artificial Sequence
  A-type consensus sequence
aattttcctgtatatgac

SEQ ID NO: 18 - DNA, 24 bp, Artificial Sequence
  B-type consensus sequence
tctaaacgtngtataggagcnnnn

SEQ ID NO: 19 - DNA, 20 bp, Artificial Sequence
  C-type consensus sequence
aggntgnntgaataagtgga

SEQ ID NO: 20 - DNA, 20 bp, Artificial Sequence
  C-prime type consensus sequence
taaaaggttgaataantgga

SEQ ID NO: 21 - DNA, 24 bp, Artificial Sequence
  Recognition sequence of homing endonuclease I-Ltr-I
tctaaacgtcgtataggagcattt

SEQ ID NO: 22 - DNA, 22 bp, Artificial Sequence
  Recognition sequence of homing endonuclease I-Onu-I
taaaaggttgaataagtggaaa

SEQ ID NO: 23 - DNA, 21 bp, Artificial Sequence
  Lsex-2R primer
ccttggccgttaaatgcggtc

SEQ ID NO: 24 - DNA, 22 bp, Artificial Sequence
  Lsex2-R-RT primer
tagacgagaagaccctatgcag

SEQ ID NO: 25 - DNA, 15 bp, Artificial Sequence
  IP2 primer
cttgcgcaaattagc

SEQ ID NO: 26 - DNA, 21 bp, Artificial Sequence
  LSEX-1 primer
gctagtagagaatacgaaggc

SEQ ID NO: 27 - DNA, 21 bp, Artificial Sequence
  LSEX-2 primer
gaccgcatttaacggccaagg

SEQ ID NO: 28 - DNA, 19 bp, Artificial Sequence
  900 FPI primer
aaattaaattctaatatgc

SEQ ID NO: 29 - DNA, 25 bp, Artificial Sequence
  254synclmap1  primer
aaagataataaagatattgtatttg

SEQ ID NO: 30 - DNA, 20 bp, Artificial Sequence
  Exon-exon junction
cgctagggataacaggctaa

SEQ ID NO: 31 - DNA, 24 bp, Artificial Sequence
  Recognition site of homing endonuclease I-LtrI -complement strand
aaatgctcctatacgacgtttaga

SEQ ID NO: 32 - DNA, 22 bp, Artificial Sequence
  Recognition site of homing endonuclease I-OnuI - complement strand
tttccacttattcaacctttta

SEQ ID NO: 33 - protein, 307 aa, Ophiostoma novo-ulmi subsp. americanum
SYSTSAYMSRRESINPWILTGFADAEGSFLLRIRNNNKSSVGYSTELGFQITLHNKDKSILENIQSTWKVGVIANSGDNAVSLKVTRFEDLKVIIDHFEKYPLITQKLGDYMLFKQAFCVMENKEHLKINGIKELVRIKAKLNWGLTDELKKAFPEIISKERSLINKNIPNFKWLAGFTSGEGCFFVNLIKSKSKLGVQVQLVFSITQHIKDKNLMNSLITYLGCGYIKEKNKSEFSWLDFVVTKFSDINDKIIPVFQENTLIGVKLEDFEDWCKVAKLIEEKKHLTESGLDEIKKIKLNMNKGRVF

SEQ ID NO: 34 - DNA, 924 bp, Artificial Sequence
  Coding sequence for I-Onu endonuclease optimized for E. Coli
agttattctacatccgcctacatgtcccgtcgcgagtccattaacccgtggattctcaccggtttcgccgacgcggaaggctcctttttgctgcgcatccgcaacaacaacaagtccagcgtcggctactccactgagctcggcttccaaattacacttcataacaaggacaagagcattcttgagaacatccagtcaacatggaaggtgggcgtgatcgccaacagcggtgacaacgccgtgtcgctgaaggtcacgcgttttgaggacctgaaggtcattatcgaccattttgaaaaatacccactgattacgcagaagctcggtgactacatgctgtttaagcaggcgttttgcgtcatggagaacaaggagcatttgaagattaatggtatcaaggagctggtgcgcattaaggcaaagctcaattggggtctgacggatgagctgaagaaggcctttccggagatcatctcgaaggagcgctccctcatcaacaagaacatccctaatttcaagtggctggcgggttttacctcgggcgagggttgcttctttgttaacctgatcaagtcaaagtcgaagctaggtgtccaggtgcagctggtgttcagcattacccaacacatcaaggataagaacctcatgaactctctgattacctacttgggctgcggctacattaaggagaaaaacaagagtgagttctcctggcttgacttcgtcgtcacgaaattctccgacatcaacgacaagatcattccggtctttcaggaaaacacgctcatcggcgtgaagctcgaggacttcgaggattggtgtaaggtcgctaagctgatcgaggagaaaaagcacctgacagaaagtggcctggacgagatcaagaagattaagctgaacatgaacaagggcagagtattctaa

SEQ ID NO: 35 - protein, 315 aa, Leptographium truncatum
SYSTLANFPVQARNDNISPWTITGFADAESSFMLTVSKDSKRNTGWSVRPRFRIGLHNKDVTILKSIREYLGAGIITSDIDARIRFESLKELEVVINHFDKYPLITQKRADYLLFKKAFYLIKNKEHLTEEGLNQILTLKASLNLGLSEELKEAFPNTIPAERLLVTGQEIPDSNWVAGFTAGEGSFYIRIAKNSTLKTGYQVQSVFQITQDTRDIELMKNLISYLNCGNIRIRKYKGSEGIHDTCVDLVVTNLNDIKEKIIPFFNKNHIIGVKLQDYRDWCKVVTLIDNKEHLTSEGLEKIQKIKEGMNRGRSL

SEQ ID NO: 36 - DNA, 948 bp, Artificial Sequence
  Coding sequence for I-LtrI endonuclease optimized for E. Coli
tcctattctactctggctaacttcccggtacaagcacgcaatgataatatcagcccatggaccattaccggcttcgcagatgcggaatcttctttcatgctgaccgtaagcaaagatagcaagcgtaacactggctggtccgttcgcccgcgttttcgtatcggcctgcataacaaagacgttaccatcctgaagtccatccgtgaatatctgggcgcgggcatcatcacttctgatatcgacgcgcgtatccgttttgaatctctgaaagaactggaagtggtaatcaaccactttgacaaatatccgctgatcactcaaaaacgcgccgactacctgctgttcaagaaagcgttctacctgatcaaaaacaaagaacacctgaccgaagaaggtctgaaccagattctgaccctgaaggcttctctgaacctgggtctgagcgaggaactgaaagaagcatttccgaacaccatcccggccgaacgcctgctggttaccggtcaggaaatcccggactccaactgggtagctggtttcactgctggtgagggttccttctacatccgtattgcgaaaaattctactctgaaaaccggctaccaggttcagtccgttttccagatcacccaggatacccgcgacatcgaactgatgaaaaacctgatctcttatctgaactgtggtaatattcgtattcgtaaatacaagggttccgagggtattcacgatacttgcgtggatctggttgttaccaacctgaacgacattaaagagaaaatcattccgttcttcaacaaaaaccacatcatcggtgtgaaactgcaggactaccgcgactggtgcaaagtggtgaccctgattgataacaaggagcatctgaccagcgaaggtctggaaaaaattcagaaaatcaaagaaggcatgaaccgtggccgttctctgtaa

(19) United States
(12) Patent Application Publication
(10) Pub. No.: US 2011/0256607 A1
(43) Pub. Date: Oct. 20, 2011

(54) HOMING ENDONUCLEASES

(76) Inventors: Georg Hausner, Winnipeg (CA); Jyothi Sethuraman, Winnipeg (CA); David Edgell, Winnipeg (CA)

(21) Appl. No.: 12/762,265

(22) Filed: Apr. 16, 2010

(51) Int. Cl. C12N 9/16; C12N 15/63; C12N 5/10; C12N 1/15; C12N 1/19; C07H 21/04; C12N 1/21

(52) U.S. Cl. 435/196; 536/23.2; 435/320.1; 435/252.3; 435/254.11; 435/254.2; 435/419; 435/348; 435/325; 435/252.33; 435/358; 435/367

(57) ABSTRACT

The present disclosure provides, in part, polypeptides having endonuclease activity, nucleic acid sequences for such a polypeptide, target sequences for the endonuclease, as well as vectors, cells, kits, methods, and uses of the same.

### TECHNICAL FIELD

The present disclosure relates to endonucleases. For example, the present disclosure relates to homing endonucleases and nucleic acid sequences, recognition sites, amino acids, proteins, vectors, cells, transgenic organisms, uses, compositions, methods, processes, and kits thereof.

### BACKGROUND

Homing endonuclease genes (HEGs) code for rare cutting DNA endonucleases. HEGs are encoded within group I or group II introns, as in-frame fusions with inteins, or as free-standing open reading frames (ORFs, Gimble 2000; Belfort et al. 2002; Toor and Zimmerly 2002). The association of HEGs with self-splicing RNA or protein elements is thought to be a mutualistic relationship, where the self-splicing elements provide the HEGs with a phenotypically neutral insertion site to minimize damage to the host genome, while the homing endonuclease (HEase) promotes mobility of the self-splicing element to related genomes (Belfort and Perlman 1995; Lambowitz et al. 1999; Schaefer 2003). In contrast, free-standing HEGs are usually found inserted in intergenic regions between genes, thus minimizing their impact on the host genome. Regardless of their insertion site, HEGs are thought to function as mobile elements by introducing a double-strand break (DSB), or nick, in genomes that lack the endonuclease coding sequence. The homing process involves host DSB-repair (DSBR) pathways that use the HEG-containing allele as a template to repair the DSB (Dujon 1989; Dujon and Belcour 1989; Belfort et al. 2002; Haugen et al. 2005; Stoddard 2005). The repair results in the nonreciprocal transfer of the HEG into the HEG-minus allele (Belfort et al. 2002).

Four families of HEase proteins have so far been described (Chevalier and Stoddard 2001). These families are designated by the presence of conserved amino acid sequence motifs: the GIY-YIG, His-Cys box, HNH, and LAGLIDADG families (Jurica and Stoddard 1999; Guhan and Muniyappa 2003). Recently, a fifth family has been recognized, an HEase encoded within a group I intron that interrupts cyanobacterial tRNA genes and that is similar to PD/E.X.K type restriction enzymes (Bonocora and Shub 2001; Zhao et al. 2007).

The LAGLIDADG endonucleases are the largest known family and are encountered in some bacteria and bacteriophages, and in organellar genomes of protozoans, fungi, plants, and sometimes in early branching Metazoans (Stoddard 2005). LAGLIDADG endonucleases typically possess one or two of the conserved LAGLIDADG amino acid sequence motifs (Chevalier and Stoddard 2001). The double-motif types are thought to have evolved by gene duplication of an ancestral single-motif HEG followed by a fusion event (Lambowitz et al. 1999; Haugen and Bhattacharya 2004). Although LAGLIDADG endonucleases may function to promote mobility, they can also function as maturases to facilitate splicing of their respective host introns (Caprara and Waring 2005).

Restriction endonucleases are frequently used to manipulate DNA for various scientific applications such as the insertion of genes in plasmid vectors for cloning and expression. The recognition site typically varies from four to eight base pairs. The shorter the recognition site sequence, and the longer the DNA to be inserted, the higher the likelihood that there will be an to internal recognition site within the segment of DNA to be cloned. Additionally, although numerous endonucleases have been isolated, many DNA sequences remain that have no cognate endonucleases and therefore are not being recognized by any known endonuclease. Also many restriction enzymes, when applied to genomic DNA, generate fragments that are too small and, consequently, are unlikely to to contain a complete gene or bacterial operon.

### SUMMARY

The present disclosure provides, in part, polypeptides having endonuclease activity, nucleic acid sequences for such a polypeptide, target sequences for the endonuclease, as well as vectors, cells, kits, methods, and uses of the same.

There is an ongoing need to obtain endonucleases having the ability to recognize and digest rare DNA sequences. And for reagents, methods, kits etc, that comprise rare-cutting endonucleases. For example, it may be desirable to limit the number of cuts an endonuclease generates within a genome, such as in characterizing bacterial mega plasmids, generating large chromosome fragments for pulse field gel electrophoresis analysis, mapping genomes, or generating vectors with a unique insertion site. For these cases the use of endonucleases that have longer recognition sites as these sites are less likely to occur frequently within most genomes may be desirable.

This summary does not necessarily describe all features of the invention. Other aspects, features and advantages of the invention will be apparent to those of ordinary skill in the art upon review of the following description of specific embodiments of the invention.

### BRIEF DESCRIPTION OF THE DRAWINGS

FIG. 1 shows an RT-PCR assay to detect splicing of the mL2449 group I intron in Ophiostoma novo-ulmi ssp americana strain WIN(M) 900. (A) Representative agarose gel of RT-PCR reactions. Lane 1 shows a PCR product (-3 kb as indicated) amplified from total DNA using primers Lsex2-R and IP2. Lane 2 is an RT-PCR reaction performed without prior reverse transcriptase step, to confirm that all DNA has been degraded. Lane 3 represents the RT-PCR product generated with primers Lsex2-R and IP2 after the reverse transcriptase step. Lanes indicated “M” are DNA size standards (1 kb plus, Invitrogen). (B) Schematic representation of the rnl region analyzed. Sequence of the RT-PCR product revealed the exon-exon junction to be 5′-CGCTAGGGAT/AACAGGCTAA-3′ (SEQ ID NO.: 30).

FIG. 2 shows a schematic representation of the mL2449 intron, the intron-encoded RPS3 gene and the HEG insertion sites. (A) Three HEG insertion sites (A, B, and C) in the RPS3 gene of ophiostomatoid fungi and related taxa. Striped rectangles indicate intron sequence, whereas the open rectangle represents the RPS3 gene. LSU (rnl), large subunit rDNA gene. (B) Example of an A-type insertion in Ophiostoma piceaperdum WIN(M)979. The shaded box indicates the LAGLIDADG HEG. (C) Example of a B-type HEG insertion in Ophiostoma europhioides WIN(M)449. (D) Example of a C-type insertion in Ophiostoma novo-ulmi subsp. americana WIN(M)900. The 4-bp direct repeats flanking the HEG are indicated by solid lines. The 52-bp spacer segment separating the HEG and downstream intron sequence is indicated by a dark box. (E) Example of an RPS3 gene with two HEG insertions in Ophiostoma laricis WIN(M)1461. The HEGs are A- and B-type insertions, as described in panels B and C, respectively.

FIG. 3 shows details of the B- and C-type HEG insertions in RPS3. Shown are HEG-minus and HEG-containing RPS3 sequences of representative Band C-type insertions, with translated amino acid sequence indicated above or below the coding-strand sequence. The dashed lines indicate the sequence that was inserted into RPS3, including the “duplicated” RPS3 sequence and the HEG. The “displaced” original RPS3 sequence is indicated by a dashed rectangle. Direct repeats flanking the C-type HEG insertion are in bold and enlarged font. There are insufficient examples of the A-type HEGs to provide details on the sequence changes that occurred during the HEG insertion.

FIG. 4 shows (A) Phylogenetic analyses of 32 double-motif LAGLIDADG sequences. Topology of trees shown in panels A and B are based on Bayesian analysis of LAGLIDADG HEase amino acid sequences. The numbers at nodes indicate the level of support based on bootstrap analysis in combination with parsimony and NJ analysis, respectively. The third number at the nodes below the line represents the posterior probability values obtained from the 50% majority consensus tree generated using Bayesian analysis. Numbers are provided for those nodes that generated high values, that is, posterior probability values of >99% and bootstrap support values >95%. NA indicates a particular node was not observed with one of the phylogenetic reconstruction methods utilized in this analysis. Accession numbers [ ] are provided for those sequences obtained by BlastP searches. (B) Phylogenetic analysis where the N- and C-terminal domains of the LAGLIDADG HEases were treated as individual sequences, nodes labeled as in panel A. The letters P and D following the HEG names indicate P=putative (i.e., HEase activity not tested) and D=degenerated (based on the presence of premature stop codons).

FIG. 5 shows the phylogenetic relationships among 47 mL2449 intron-encoded Rps3 amino acid sequences. Tree topology is based on a 50% majority consensus tree generated using Bayesian analysis (Ronquist et al. 2003; Ronquist 2004). Among the 34 Ophiostoma and Leptographium Rps3 sequences used, 24 had HEG insertions and 11 sequences (denoted by *) had no HEG insertions. Rps3 sequences marked with (+) had remnants of degenerate LAGLIDADG ORFs and were not included in the HEG phylogenies (FIGS. 4A and B). Nodes, with regard to statistical support, were labeled as in FIG. 4. On the right side of the phylogenetic tree is a table indicating the presence/absence of HEGs inserted in RPS3 genes for each species. The sizes of the IP1/IP2 PCR products obtained are indicated (short [S]=1.55 kb and long [L]>2.4 kb). L indicates the presence and S the absence of HEGs within RPS3. The HEG insertion positions are indicated by either A, B, or C (see FIG. 2). Any evidence for ORF degeneration (i.e., premature stop codons, frameshift mutations) is indicated by YES and the absence of degeneration by NO.

FIG. 6 shows the purification and characterization of I-OnuI. (A) “Top gel,” SDS-PAGE analysis of I-OnuI purification by HisTrapHP. Lanes are indicated as follows: U, uninduced cells; I, induced cells; C, crude fraction from induced cells; P, insoluble fraction; S, soluble fraction; FT, flow through; W, wash. I-OnuI was eluted over an increasing linear gradient of immidazole as indicated by the left-facing triangle. “Bottom gel,” 6% SDS-gel showing the peak fractions from Superdex 75 gel-filtration column, with fraction numbers indicated above the gel. (B) In vitro cleavage assay with I-InuI. Lane 1, uncut pRPS3; lane 2, pRPS3 linearized with PstI; lanes 3-5, cleavage assays with pRPS3 incubated for 0, 15, and 30 min with I-OnuI; lane 6, cleavage assay with pRPS3+HEG construct; lane 7, cleavage assay with pU7143 (mL1669 intron with ORF). The lane marked M is the 1-kb-plus Ladder. (C) Physical map of the pRPS3 used for generating substrate molecules via PCR for cleavage mapping assays. In the diagram, open boxes outline the RPS3 gene. Shown are relative positions of primers (IP1, IP2, 900FP1) used to generate substrate for mapping, with the position of the GAAT insertion site noted. (D) Mapping of I-OnuI cleavage sites. Shown is a representative gel where end-labeled PCR products (=SUB for substrate) corresponding to the coding (top) or noncoding (bottom) strands were incubated with I-OnuI (+) or with buffer (−). Cleavage products (=CP) were electrophoresed alongside the corresponding sequencing ladders. Schematic representation of the I-OnuI cleavage sites, indicated by solid triangles on the top strand and bottom strand. The HEG insertion site based on comparative sequence analysis would be after the GAAT.

FIG. 7 shows the mapping of I-LtrI cleavage sites. Shown is a representative gel where end-labeled PCR products (=SUB for substrate) corresponding to the coding (top) or noncoding (bottom) strands were incubated with I-LtrI (+) or with buffer only (−). Cleavage products (=CP) were electrophoresed alongside the corresponding DNA sequencing ladders. Shown below is a schematic representation of the I-LtrI cleavage sites, indicated by solid triangles on the top strand and bottom strand; insertion site for HEG is also noted by a vertical line.

FIG. 8(A) shows sequence logos (Schneider and Stephens 1990) representing those segments of the Rps3 amino acid alignments corresponding to nucleotide positions that are invaded by HEGs at the gene level. Vertical lines indicated the three Rps3 HEG insertion sites: A, B, and C. The sequence logos were generated using the online program WebLogo (Crooks et al. 2004).(B) The relative HEG insertion points with regard to the Rps3 amino acid sequence are shown with reference to the Rps3 amino acids sequence obtained from Ophiostom novo-ulmi subsp. americana strain WIN(M) 904 (a HEG-minus allele; GenBank accession: AY275137). (C). Structure of Escherichia coli Rps3 protein with the position of the B- and C-type HEG insertion sites in the corresponding fungal Rps3 denoted by arrows (modified from PDB 1FKA; Schluenzen et al. 2000). Details of A-type insertions were not shown as the intron-encoded version of Rps3 appears to have no similarity with the N-terminal region of the bacterial type Rps3.

FIG. 9(A) shows the recognition site for I-LtrI HEase (SEQ ID NO: 21) and the location of cleavage. (B) shows the recognition site for I-OnuI HEase (SEQ ID NO: 22) and the location of cleavage.

FIG. 10(A) shows the sequence of SEQ ID NO: 1. (B) shows the sequence of SEQ ID NO: 2. (C) shows the sequence of SEQ ID NO: 3. (D) shows the sequence of SEQ ID NO: 4. (E) shows the sequence of SEQ ID NO: 5. (F) shows the sequence of SEQ ID NO: 6. (G) shows the sequence of SEQ ID NO: 7. (H) shows the sequence of SEQ ID NO: 8. (I) shows the sequence of SEQ ID NO: 9. (J) shows the sequence of SEQ ID NO: 10. (K) shows the sequence of SEQ ID NO: 11. (L) shows the sequence of SEQ ID NO: 12. (M) shows the sequence of SEQ ID NO: 13. (N) shows the sequence of SEQ ID NO: 14. (O) shows the sequence of SEQ ID NO: 15. (P) shows the sequence of SEQ ID NO: 16. (Q) shows the sequence of SEQ ID NO: 33. (R) shows the sequence of SEQ ID NO: 34. (S) shows the sequence of SEQ ID NO: 35. (T) shows the sequence of SEQ ID NO: 36.

### DETAILED DESCRIPTION

The present disclosure provides, in part, homing endonuclease (HEase) nucleic acid molecules and polypeptides that can be used to cleave specific double-stranded DNA sequences. The disclosure also relates, in part, to vectors comprising such sequences, transformed cells, cell lines, and transgenic organisms. The present disclosure also provides methods for producing HEase polypeptides. The present disclosure further relates to a method for site-directed homologous recombination, a method of inserting a nucleic acid into a target nucleic acid, and a method of deleting a nucleic acid from a target nucleic acid. The present disclosure provides compositions, uses, and kits comprising homing endonucleases.

In the description that follows, a number of terms are used extensively, the following definitions are provided to facilitate understanding of various aspects of the invention. Use of examples in the specification, including examples of terms, is for illustrative purposes only and is not intended to limit the scope and meaning of the embodiments of the invention herein.

Any terms not directly defined herein shall be understood to have the meanings commonly associated with them as understood within the art of the invention. Certain terms are discussed below, or elsewhere in the specification, to provide additional guidance to the practitioner in describing the devices, methods and the like of embodiments of the invention, and how to make or use them. It will be appreciated that the same thing may be said in more than one way. Consequently, alternative language and synonyms may be used for any one or more of the terms discussed herein. No significance is to be placed upon whether or not a term is elaborated or discussed herein. Some synonyms or substitutable methods, materials and the like are provided. Recital of one or a few synonyms or equivalents does not exclude use of other synonyms or equivalents, unless it is explicitly stated. Use of examples in the specification, including examples of terms, is for illustrative purposes only and does not limit the scope and meaning of the embodiments of the invention herein.

The present disclosure relates to one, or more than one, HEase nucleic acid molecule and one, or more than one, HEase polypeptide.

The term “homing endonuclease” or “HEase” as used herein, refers to endonucleases that are capable of recognizing a specific nucleotide sequence (recognition site) in a deoxyribonucleic acid (DNA) molecule and cleaving the DNA at specific sites. The recognition sites for HEases are typically 10bp of greater, 12bp or greater, l4bp or greater, 16bp or greater, 18bp or greater.

The terms “DNA target”, “DNA target sequence”, “target sequence”, “target”, “recognition site”, “recognition sequence”, “homing recognition site”, “homing site”, “homing site sequence”, “cleavage site” “site-specific sequence” are intended to mean a double-stranded palindromic, partially palindromic (pseudo-palindromic) or non-palindromic nucleotide sequence that is recognized and cleaved by a HEase. These terms refer to a distinct DNA location at which a double-stranded break (cleavage) is to be induced by the endonuclease. The DNA target is defined by the 5′ to 3′ sequence of one strand of the double-stranded nucleotide.

In the context of this application, the term “nucleotide” includes DNA conventionally having adenine, cytosine, guanine and thymine as bases and deoxyribose as the structural sugar element. Furthermore, a nucleotide can, however, also comprise any modified base known to the skilled artisan, which is capable of base pairing using at least one of the aforesaid bases. Further included in the term “nucleotide” are the derivatives of the aforesaid compounds, in particular derivatives being modified with dyes or radioactive markers. Conventional designation for the following nucleotides are used: A for Adenine, G for Guanine, T for Thymine and C for Cytosine.

“Nucleic acid” used herein may mean any nucleic acid containing molecule including, but not limited to, DNA or RNA. The depiction of a single strand also defines the sequence of the complementary strand. Thus, a nucleic acid also encompasses the complementary strand of a depicted single strand. A nucleic acid may be single stranded or double stranded, or may contain portions of both double stranded and single stranded sequence. The nucleic acid may be DNA, both genomic and cDNA, RNA, or a hybrid, where the nucleic acid may contain combinations of deoxyribo- and ribo-nucleotides, and combinations of bases including uracil, adenine, thymine, cytosine, guanine, inosine, xanthine hypoxanthine, isocytosine and isoguanine. Nucleic acids may be obtained by chemical synthesis methods or by recombinant methods.

The terms “peptide”, “polypeptide” or “protein” as used herein, refers to a string of at least three amino acids linked together by peptide bonds. The present peptides preferably contain only natural amino acids, although non-natural amino acids (i.e., compounds that do not occur in nature but that can be incorporated into a polypeptide chain) and/or amino acid analogs as are known in the art may alternatively be employed. Also, one or more of the amino acids may be modified, for example, by the addition of a chemical entity such as a carbohydrate group, a phosphate group, a farnesyl group, an isofarnesyl group, a fatty acid group, a linker for conjugation, functionalization, or to other modification (e.g., alpha amindation), etc.

The term “vector” as used herein refers to a nucleic acid molecule, such as DNA, used as a vehicle to transfer foreign genetic material into a cell. Major types of vectors include plasm ids, bacteriophages and other viruses, cosmids, and artificial chromosomes. The vector is generally DNA sequence that consists of an insert (transgene) and a larger sequence that serves as the “backbone” of the vector. Expression vectors are utilized for the expression of the transgene in a target cell, and generally have a promoter sequence that drives expression of the transgene. Simpler vectors called transcription vectors are only capable of being transcribed but not translated.

One, or more than one, nucleic acid encoding a HEase are provided. The one, or more than one, nucleic acid may comprise the sequence set forth in SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 34, SEQ ID NO: 36, combinations thereof, or sequences substantially similar thereto. The sequence of the nucleic acid may be changed, for example, to account for codon preference in a particular host cell. The nucleic acid may be synthesized or derived from a fungi such as Ophiostoma and related taxa, such as Ophiostoma novo-ulmi subsp americana (WIN(M) 900), Ophiostoma penicillatum (WIN(M) 27), Ophiostoma piceaperdum (WIN(M) 979), Ophiostoma ulmi (WIN(M) 1223), Leptographium pithyophilum (WIN(M) 1454), Leptographium truncatum (WIN(M) 1434), L. truncatum (WIN(M) 254), Sporothrix sp. (WIN(M) 924) using standard molecular biology techniques.

The present disclosure provides a nucleic acid encoding for I-LtrI (SEQ ID NO: 36), or an active fragment thereof, which is derived from Leptographium truncatum.

The present disclosure provides a nucleic acid encoding for I-OnuI (SEQ ID NO: 34), or an active fragment thereof, which is derived from Ophiostoma novo-ulmi subsp americana.

The present disclosure provides nucleic acid sequences encoding for a polypeptide having a sequence selected from SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 33, SEQ ID NO: 35, or sequences substantially identical thereto. The present disclosure provides nucleic acid sequences encoding for a polypeptide having a sequence selected from SEQ ID NO: 1, SEQ ID NO: 3, SEQ ID NO: 5, SEQ ID NO: 7, SEQ ID NO: 11, SEQ ID NO: 13, SEQ ID NO: 15, SEQ ID NO: 33, SEQ ID NO: 35, or sequences substantially identical thereto.

This disclosure includes variants of the nucleic acid sequences of the invention exhibiting substantially the same properties as the sequences of the invention. By this it is meant that nucleic acid sequences need not be identical to the sequence disclosed herein. Variations can be attributable to single or multiple base substitutions, deletions, or insertions or local mutations involving one or more nucleotides not substantially detracting from the properties of the nucleic acid sequence as encoding an enzyme having the cleavage properties of the HEase of the invention.

The present disclosure provides a synthetic gene comprising one or more than one nucleic acid encoding HEase, the nucleic acid operably linked to a transcriptional or translational regulatory sequence or both. The synthetic gene may be capable of expressing the HEase polypeptide. The synthetic gene may also comprise terminators at the 3′-end of the transcriptional unit of the synthetic gene sequence. The synthetic gene may also comprise a selectable marker.

The present disclosure provides one or more than one nucleic acid comprising a HEase recognition site or a consensus sequence for a HEase recognition site.

As used herein, the term “consensus sequence” means an idealized sequence that represents the nucleotides most often present at each position in a given segment of all members of the family of recognition sequences. One method of determining a consensus sequence known in the art is to use a computer program to compare the target nucleic acid sequence and all its family member sequences for which a consensus sequence is desired.

The recognition site may have an A-type Consensus Sequence:

| | |
|---|---|
| 5′ AATTTTCCTGTATATGAC 3′ | (SEQ ID NO: 17) |

The recognition site may have a B-type Consensus Sequence:

5′ TCTAAACGTN₁GTATAGGAGCNNNN 3′ (SEQ ID NO: 18), wherein N₁ might be C or A and N might be A, G, C or T.

The recognition site may have a C-type consensus sequence:

5′ AGGN₁TGN₂N₃TGAATAMTGGA 3′ (SEQ ID NO: 19), wherein N₁ might be T or A, N₂ might be A or G and N₃ might be A or T.

The recognition site may have a C′-type consensus sequence:

5′ TAAAAGGTTGAATAAN ₁TGGA 3′ (SEQ ID NO: 20), wherein N₁ might be T or G.

The nucleic acid sequence comprising a HEase consensus recognition site may be selected from SEQ ID NO: 17, SEQ ID NO: 18, SEQ ID NO: 19, SEQ ID NO: 20, or a combination thereof, or sequences substantially identical thereto.

The present HEases, in particular I-Ltr-I, may recognize and cleave a target double-stranded DNA at a specific recognition site according to the following cutting pattern:

| | |
|---|---|
| 5′ TCTAAACGTC GTAT\|AGGAGCATTT 3′ | (SEQ ID NO: 21) |
| 3′ AGATTTGCAG\|CATA TCCTCGTAAA 5′ | (SEQ ID NO: 31) |

where | denotes the top- and bottom-strand cleavage sites, respectively. 3′ four nucleotide overhang (GTAT) is underlined.

The present HEases, in particular I-Onu-I, may recognize and cleave a target double-stranded DNA at a specific recognition site according to the following cutting pattern:

| | |
|---|---|
| *5′ TAAAAGGTT GAAT\|AAGTGGAAA 3′* | (SEQ ID NO: 22) |
| *3′ ATTTTCCAA\|CTTA TTCACCTTT 5′* | (SEQ ID NO: 32) |

where | denotes the top- and bottom-strand cleavage sites, respectively. 3′ four nucleotide overhang (GAAT) is underlined.

The HEase recognition site may comprise the sequence set forth in SEQ ID NO: 21 or SEQ ID NO: 22, or sequences substantially identical thereto.

“Identical” or “identity” used herein in the context of two or more nucleic acids, may mean that the sequences have a specified percentage of residues that are the same over a region of comparison. The percentage may be calculated by optimally aligning the two sequences, comparing the two sequences over the specified region, determining the number of positions at which the identical residue occurs in both sequences to yield the number of matched positions, dividing the number of matched positions by the total number of positions in the region of comparison, and multiplying the result by 100 to yield the percentage of sequence identity. In cases where the two sequences are of different lengths or the alignment produces one or more staggered ends and the specified region of comparison includes only a single sequence, the residues of single sequence may be included in the denominator but not the numerator of the calculation. When comparing DNA and RNA, thymine (T) and uracil (U) may be considered equivalent. Identity may be performed manually or by using a computer sequence algorithm such as BLAST or BLAST 2.0.

Also provided are one, or more than one HEase polypeptides. The one, or more than one HEase polypeptides may comprise the sequence set forth in SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 34, SEQ ID NO: 36, or sequences having at least about 80-100% sequence similarity thereto, including any percent similarity within these ranges, such as 81, 82, 83, 84, 85, 86, 87, 88, 89, 90, 91, 92, 93, 94, 95, 96, 97, 98, 99% sequence similarity thereto.

A substantially similar sequence is an amino acid sequence that differs from a reference sequence only by one or more conservative substitutions. Such a sequence may, for example, be functionally homologous to another substantially similar sequence. It will be appreciated by a person of skill in the art the aspects of the individual amino acids in a peptide of the invention that may be substituted.

Amino acid sequence similarity or identity may be computed by using the BLASTP and TBLASTN programs which employ the BLAST (basic local alignment search tool) 2.0 algorithm. Techniques for computing amino acid sequence similarity or identity are well known to those skilled in the art, and the use of the BLAST algorithm is described in ALTSCHUL et al. 1990, J Mol. Biol. 215: 403-410 and ALTSCHUL et al. (1997), Nucleic Acids Res. 25: 3389-3402.

Standard reference works setting forth the general principles of peptide synthesis technology and methods known to those of skill in the art include, for example: Chan et al., Fmoc Solid Phase Peptide Synthesis, Oxford University Press, Oxford, United Kingdom, 2005; Peptide and Protein Drug Analysis, ed. Reid, R., Marcel Dekker, Inc., 2000; Epitope Mapping, ed. Westwood et al., Oxford University Press, Oxford, United Kingdom, 2000; Sambrook et al., Molecular Cloning: A Laboratory Manual, 3^{rd} ed., Cold Spring Harbor Press, Cold Spring Harbor, N.Y. 2001; and Ausubel et al., Current Protocols in Molecular Biology, Greene Publishing Associates and John Wiley & Sons, NY, 1994).

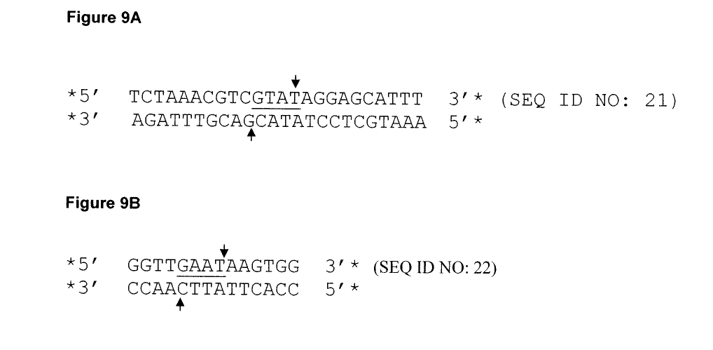

The one, or more than one, HEase polypeptide may be an endonuclease that cleaves a HEase recognition site. In some embodiments, the HEase polypeptide recognizes and cleaves a consensus recognition site comprising a nucleotide sequence selected from the group consisting of SEQ ID NO: 17, SEQ ID NO: 18, SEQ ID NO: 19, and SEQ ID NO: 20, or sequences substantially identical thereto. In certain embodiments the recognition site may comprise the sequence set forth in SEQ ID NO: 21 or SEQ ID NO: 22 and the recognition site may be cleaved as indicated in FIG. 9A for SEQ ID NO. 21 and FIG. 9B for SEQ ID NO. 22.

The HEase polypeptide may be a fusion protein comprising a polypeptide or peptide which may be used to purify the HEase polypeptide. Representative examples of such peptides include a histidine tag, a maltose-binding protein fusion or a chitin-binding intein fusion.

Also provided is a method of cleaving a target nucleic acid comprising a HEase recognition site. A target nucleic acid comprising a HEase recognition site may be contacted with a HEase polypeptide under conditions that allow cleavage of the recognition site. The recognition site may have a consensus sequence.

The target nucleic acid may comprise the HEase recognition site selected from the group consisting of SEQ ID NO: 17, SEQ ID NO: 18, SEQ ID NO: 19, SEQ ID NO: 20, SEQ ID NO:21, and SEQ ID NO: 22, or sequences substantially identical thereto.

The target nucleic acid may be cleaved in vitro or in vivo. The recognition site may be present in a linear or circular target nucleic acid. The target nucleic acid may be a plasmid or a chromosome. The recognition site may be a naturally occurring site in the target nucleic acid or may be introduced into the target nucleic acid by methods including, but not limited to, mutagenesis (e.g., site-directed or cassette), homologous recombination or transposition.

The disclosure also relates, in part, to cloning and expression vectors comprising the nucleic acid encoding for a HEase polypeptide. Provided is a vector comprising one or more than one HEase nucleic acid or synthetic HEase gene. The vector may be a cloning vector. The vector may also be an expression vector, wherein the one or more than one HEase nucleic acid or synthetic HEase gene are placed under control of appropriate transcriptional and translational control elements to permit production or synthesis of the HEase polypeptide. Therefore, the one or more than one HEase nucleic acid or synthetic HEase gene are comprised in expression cassettes. The vector may comprise a replication origin, a promoter operatively linked to the one or more than one HEase nucleic acid or synthetic HEase gene encoding the HEase polypeptide, a ribosome-binding site, an RNA-splicing site (when genomic DNA is used), a polyadenylation site, and a transcription termination site. It may also comprise an enhancer. Selection of the promoter will depend upon the cell in which the polypeptide is expressed.

The vector may comprise two replication systems allowing it to be maintained in two organisms, e.g., in one host cell for expression and in a second host cell (e.g., bacteria) for cloning and amplification. For integrating expression vectors, the expression vector may comprise a sequence homologous to a host cell genome, such as two homologous sequences which flank the expression construct. The integrating vector may be directed to a specific locus in the host cell by selecting the appropriate homologous sequence for inclusion in the vector.

The vector may comprise additional elements. The vector may also comprise a selectable marker gene to allow the selection of transformed host cells for example: neomycin phosphotransferase, histidinol dehydrogenase, dihydrofolate reductase, hygromycin phosphotransferase, herpes simplex virus thymidine kinase, adenosine deaminase, glutamine synthetase, or hypoxanthine-guanine phosphoribosyl transferase for eukaryotic cell culture; TRP1 for S. cerevisiae; tetracycline, rifampicin or ampicillin resistance in E. coli.

One type of preferred vector is an episome, i.e., a nucleic acid capable of extra-chromosomal replication. Preferred vectors are those capable of autonomous replication or expression of nucleic acids to which they are linked. A vector according to the present disclosure comprises, but is not limited to, a YAC (yeast artificial chromosome), a BAC (bacterial artificial), a baculovirus vector, a phage, a phagemid, a cosmid, a viral vector, a plasmid, a RNA vector or a linear or circular DNA or RNA molecule which may consist of chromosomal, non chromosomal, semi-synthetic or synthetic DNA. In general, expression vectors of utility in recombinant DNA techniques are often in the form of “plasmids” which refer generally to circular double-stranded DNA loops which, in their vector form are not bound to the chromosome.

The present vector may comprise one, or more than one, nucleic acid sequence selected from SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 33, SEQ ID NO: 35, or a sequence substantially identical thereto.

The present vector may comprise one, or more than one, nucleic acid sequence encoding a polypeptide having a sequence selected from SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 34, SEQ ID NO: 36, or a sequence substantially identical thereto. The present vector may comprise one, or more than one, nucleic acid sequence encoding a polypeptide having a sequence selected from SEQ ID NO: 1, SEQ ID NO: 3, SEQ ID NO: 5, SEQ ID NO: 7, SEQ ID NO: 11, SEQ ID NO: 13, SEQ ID NO: 15, SEQ ID NO: 34, SEQ ID NO: 36, or a sequence substantially identical thereto.

The present vector may comprise one, or more than one, nucleic acid sequences encoding a HEase polypeptide that cleaves a recognition site comprising a nucleotide sequence selected from SEQ ID NO: 17, SEQ ID NO: 18, SEQ ID NO: 19, SEQ ID NO: 20, SEQ ID NO: 21 or SEQ ID NO: 22, or a sequence substantially identical thereto.

Also provided is a vector comprising a HEase recognition site. The vector may comprise a nucleic acid of interest with the HEase recognition site within or adjacent to the nucleic acid of interest. The nucleic acid of interest may encode a polypeptide.

The present recognition site may comprise a sequence selected from SEQ ID NO: 17, SEQ ID NO: 18, SEQ ID NO: 19, SEQ ID NO: 20, SEQ ID NO:21, SEQ ID NO: 22, or a sequence substantially identical thereto.

The present disclosure provides a vector comprising one, or more than one, nucleic acid sequence encoding a HEase polypetide and/or a HEase recognition site.

The disclosure also provides a prokaryotic or eukaryotic host cell which is modified by a polynucleotide or a vector as defined herein. The host cell may comprise a HEase vector, synthetic HEase gene, and/or HEase nucleic acid. The host cell may be any cell that is capable of being transformed by the vector, synthetic gene, and/or nucleic acid. The host cell may also be any cell that is capable of expressing the HEase polypeptide.

Also provided is a host cell into which the HEase recognition site has been introduced. The host cell may comprise a nucleic acid of interest with the HEase recognition site within or adjacent to the nucleic acid of interest. The nucleic acid may encode a polypeptide. The HEase recognition site may be on a vector in the host cell. The HEase recognition site may also be introduced onto a chromosome of the host cell.

The host cell may comprise a HEase vector, synthetic HEase gene, and/or HEase nucleic acid and a nucleic acid of interest with the HEase recognition site within or adjacent to the nucleic acid of interest.

The vector may be obtained and introduced in a host cell by well-known recombinant DNA and genetic engineering techniques. The one or more than one polynucleotide sequence encoding the HEase as defined in the present disclosure may be prepared by any method known by the person skilled in the art. For example, they may be amplified from a cDNA template, by polymerase chain reaction with specific primers. Preferably the codons of said cDNA are chosen to favour the expression of said protein in the desired expression system.

The host cell may be prokaryotic, such as bacterial, or eukaryotic, such as fungal (e.g., yeast), plant, insect, amphibian or animal cell. Representative examples of a bacterial host cell include, but are not limited to, E. coli strains such as ER2566. Representative examples of a mammalian host cell include CHO and HeLa cells.

Also provided is a method of transforming a host cell with the HEase vector, synthetic HEase gene, and/or HEase nucleic acid, or a vector comprising the HEase recognition site or HEase recognition site nucleic acid. The host cell may be contacted with the vector, synthetic gene, or nucleic acid under conditions that allow transformation of the host cell. The host cell may be transformed by methods including, but not limited to, transformation, transfection, electroporation, microinjection, or by means of liposomes (lipofection). The transformed cell may be selected, for example, by selecting for a selectable marker on the vector, synthetic gene or nucleic acid.

Also provided is a method of producing the HEase polypeptide. A host cell comprising the HEase vector, synthetic HEase gene, and/or HEase nucleic acid that is capable of expressing HEase may be provided. The host cell may be incubated under conditions that allow expression of the HEase polypeptide. The HEase polypeptide may be purified using standard chromatographic techniques.

Also provided is a HEase kit. The kit may comprise one or more HEase nucleic acid molecules. The kit may comprise one or more HEase polypeptides. The kit may comprise a synthetic HEase gene. The kit may comprise a vector comprising one or more HEase nucleic acids. The kit may comprise a vector comprising the HEase recognition site. The kit may comprise a host cell capable of expressing one or more than one HEase polypeptide. The kit may comprise a host cell comprising one or more than one HEase recognition site. In certain embodiments, the kit is provided for therapeutic purposes. For example, the kit may be used to design and/or evolve a therapeutic construct which is then introduced into a subject or cells of the subject, which then may be introduced into the subject. The cells may preferably be blood cells, bone marrow cells, stem cells, or progenitor cells. The kit may also include a vector for introducing the construct into cells.

The HEase polypeptide according to the disclosure may also be used in a variety of other applications. Such applications include, without limitation, site specific gene insertion, site specific gene expression and a variety of biomedical applications, such as repairing, modifying, attenuating, inactivating or mutating a specific sequence.

The ability to cleave HEase recognition sites in vivo without detriment to the host cell allows HEase to be used in a number of techniques for the modification of nucleic acids (e.g., chromosomal and plasmid) within a host cell. For example, HEase may be used to induce the introduction of a double-strand break at a HEase recognition site in a target nucleic acid, such as a plasmid or a chromosome. The double-strand break in the target nucleic acid may also induce homologous recombination within the target nucleic acid (intrastrand homologous recombination) or between the target nucleic acid and another nucleic acid (interstrand homologous recombination). The homologous recombination may lead to the insertion or deletion of a portion of a nucleic acid (e.g., a gene). The nucleic acid may encode a polypeptide.

Site specific gene insertion methods allow the production of an unlimited number of cells and cell lines in which various genes or mutants of a given gene can be inserted at the predetermined location defined by the previous integration of the HEase recognition site. Such cells and cell lines are thus useful for screening procedures, for phenotypes, ligands, drugs and for reproducible expression.

Above cell lines are initially created with the HEase recognition site being heterozygous (present on only one of the two homologous chromosomes). They can be propagated as such or used to create transgenic animals or both. In such case, homozygous transgenics (with HEase recognition site sites at equivalent positions in the two homologous chromosomes) can be constructed by regular methods such as mating. Homozygous cell lines can be isolated from such animals. Alternatively, homozygous cell lines can be constructed from heterozygous cell lines by secondary transformation with appropriate DNA constructs. It is also understood that cell lines containing compensated heterozygous HEase insertions at nearby sites in the same gene or in neighbouring genes are part of this disclosure.

Mouse cells or equivalents from other vertebrates, including man, can be used. Cells from invertebrates can also be used. Any plant cells that can be maintained in culture can also be used independently of whether they have ability to regenerate or not, or whether or not they have given rise to fertile plants. The methods can also be used with transgenic animals.

Cell lines can also be used to produce proteins, metabolites, or other compounds of biological or biotechnological interest using a transgene, a variety of promoters, regulators, and/or structural genes. The gene will be always inserted at the same localisation in the chromosome. In transgenic animals, it makes possible to test the effect of multiple drugs, ligands, or medical proteins in a tissue-specific manner.

The HEase recognition site and HEase polypeptide can also be used in combination with homologous recombination techniques, well known in the art. It is understood that the inserted sequences can be maintained in a heterozygous state or a homozygous state. In cases of transgenic animals with the inserted sequences in a heterozygous state, homozygation can be induced, for example, in a tissue specific manner, by induction of HEase expression from an inducible promoter.

The insertion of a HEase recognition site into the genome by spontaneous homologous recombination can be achieved by the introduction of a plasmid construct containing the HEase recognition site and a sequence sharing homology with a chromosomal sequence in the targeted cell. The input plasmid is constructed recombinantly with a chromosomal target. This recombination may lead to a site-directed insertion of at least one HEase recognition site into the chromosome. The targeting construct can either be circular or linear and may contain one, two, or more parts of sequence that is homologous to a sequence contained in the targeted cell. The targeting mechanism can occur either by the insertion of the plasmid construct into the target or by the replacement of a chromosomal sequence by a sequence containing the HEase recognition site.

The chromosomal target locus can be exons, introns, promoter regions, locus control regions, pseudogenes, retroelements, repeated elements, non-functional DNA, telomers, and minisatellites. The targeting can occur at one locus or multiple loci, resulting in the insertion of one or more HEase recognition sites into the cellular genome.

The use of embryonic stem cells for the introduction of the HEase recognition sites into a precise locus of the genome allow, by the reimplantation of these cells into an early embryo (amorula or a blastocyst stage), the production of mutated animals containing the HEase recognition site at a precise locus. These animals can be used to modify their genome in expressing the HEase polypeptide into their somatic cells or into their germ line.

There are various applications where the sequences, vectors, cells, animals, chromosomes, compositions, uses and methods according to the disclosure may be useful.

One application is gene therapy. Specific examples of gene therapy include immunomodulation (i.e. changing range or expression of IL genes); replacement of defective genes; and excretion of proteins (i.e. expression of various secretory protein in organelles).

The present disclosure further embodies transgenic organisms, for example animals, where an HEase restriction site is introduced into a locus of a genomic sequence or in a part of a cDNA corresponding to an exon of the gene. Any gene (animal, human, insect, plant, etc.) in which a HEase recognition site is introduced can be targeted by a plasmid containing the sequence encoding the corresponding endonuclease. Introduction of a HEase recognition site may be accomplished by homologous recombination. Thus, any gene can be targeted to a specific location for expression.

It may be possible to activate a specific gene in vivo by HEase induced recombination. The HEase cleavage site may be introduced between a duplication of a gene in tandem repeats, creating a loss of function. Expression of the HEase polypetide can induce the cleavage of the two copies. The repair by recombination can be stimulated and result in a functional gene.

Specific translocation of chromosomes or deletion can be induced by HEase cleavage. Locus insertion can be achieved by integration of one at a specific location in the chromosome by “classical gene replacement.” The cleavage of recognition sequence by HEase can be repaired by non-lethal translocations or by deletion followed by end-joining. A deletion of a fragment of chromosome may also be obtained by insertion of two or more HEase sites in flanking regions of a locus. The cleavage can be repaired by recombination and result in deletion of the complete region between the two sites.

The present disclosure also relates, in part, to a method for significantly increasing the frequency of homologous recombination and D-loop recombination-mediated gene repair (see U.S. Pat. No. 7,285,538, the contents of which are hereby incorporated by reference). Application of such method include, without limitation, repairing, modifying, attenuating, inactivating, or mutating a specific sequence. Methods further include, for example, treating or prophylaxis of a genetic disease. Methods include the generation of animal models.

The disclosure also relates, in part, to the use of methods which lead to the excision of homologous targeting DNA sequences from a recombinant vector within transfected cells (cells which have taken up the vector). The methods comprise introducing into cells (a) a first vector which comprises a targeting DNA, wherein the targeting DNA flanked by HEase recognition site(s) and comprises DNA homologous to a chromosomal target site, and (b) a restriction endonuclease which cleaves the HEase recognition site(s) present in the first vector or a second vector which comprises a nucleic acid encoding the HEase. Alternatively, a vector which comprises both targeting DNA and a nucleic acid encoding a HEase which cleaves the HEase recognition site(s) is introduced into the cell.

The present disclosure relates to a method of repairing a specific sequence of interest in chromosomal DNA of a cell comprising introducing into the cell (a) a vector comprising targeting DNA, wherein the targeting DNA is flanked by a HEase recognition site or sites and comprises (1) DNA homologous to chromosomal DNA adjacent to the specific sequence of interest and (2) DNA which repairs the specific sequence of interest upon recombination between the targeting DNA and the chromosomal DNA, and (b) a HEase which cleaves the HEase recognition site(s) present in the vector. Preferably, the targeting DNA is flanked by two HEase recognition sites (one at or near each end of the targeting DNA). In another embodiment of this method, the restriction endonuclease is introduced into the cell by introducing into the cell a second vector which comprises a nucleic acid encoding a HEase which cleaves the HEase recognition site(s) present in the vector. In yet another embodiment of this method, both targeting DNA and nucleic acid encoding the HEase are introduced into the cell in the same vector.

The present disclosure also relates to a method of modifying a specific sequence (e.g a gene) in chromosomal DNA of a cell comprising introducing into the cell (a) a vector comprising targeting DNA, wherein the targeting DNA is flanked by a HEase recognition site and comprises (1) DNA homologous to the specific sequence to be modified and (2) DNA which modifies the specific sequence upon recombination between the targeting DNA and the chromosomal DNA, and (b) a HEase which cleaves the H Ease recognition site present in the vector. Preferably, the targeting DNA is flanked by two HEase recognition sites. In another embodiment of this method, the HEase is introduced into the cell by introducing into the cell a second vector (either RNA or DNA) which comprises a nucleic acid encoding the HEase. In yet another embodiment of this method, both targeting DNA and nucleic acid encoding the HEase are introduced into the cell in the same vector.

The disclosure further relates to a method of attenuating or inactivating an endogenous gene of interest in a cell comprising introducing into the cell (a) a vector comprising targeting DNA, wherein the targeting DNA is flanked by a HEase recognition site and comprises (1) DNA to homologous to a target site of the endogenous gene of interest and (2) DNA which attenuates or inactivates the gene of interest upon recombination between the targeting DNA and the gene of interest, and (b) a HEase which cleaves the restriction endonuclease site present in the vector. Preferably, the targeting DNA is flanked by two HEase recognition sites, as described above. In another embodiment of this method, the HEase is introduced into the cell by introducing into the cell a second vector (either RNA or DNA) which comprises a nucleic acid encoding the HEase. In yet another embodiment of this method, both the targeting DNA and the nucleic acid encoding the HEase are introduced into the cell in the same vector.

The present disclosure also relates to a method of introducing a mutation into a target site (or gene) of chromosomal DNA of a cell comprising introducing into the cell (a) a first vector comprising targeting DNA, wherein the targeting DNA is flanked by a restriction endonuclease site and comprises (1) DNA homologous to the target site (or gene) and (2) the mutation to be introduced into the chromosomal DNA, and (b) a second vector (RNA or DNA) comprising a nucleic acid encoding a HEase which cleaves the HEase recognition site present in the first vector. Preferably, the targeting DNA is flanked by two restriction endonuclease sites. In another embodiment of this method, the HEase is introduced directly into the cell. In yet another embodiment of this method, both targeting DNA and nucleic acid encoding a HEase which cleaves the HEase recognition site, are introduced into the cell in the same vector.

The disclosure further relates to a method of treating or prophylaxis of a genetic abnormality in an individual in need thereof. As used herein, a genetic abnormality refers to a disease or disorder that arises as a result of a genetic defect (mutation) in a gene in the individual. The term also refers to genetic defects that are asymptomatic in the individual but may cause disease or disorder in off-spring. The genetic abnormality may arise as a result of a point mutation in a gene in the individual.

In one embodiment, the method of treating or prophylaxis of a genetic abnormality in an individual in need thereof comprises introducing to the individual (a) a first vector comprising targeting DNA, wherein the targeting DNA is flanked by a HEase recognition site(s) and comprises (1) DNA homologous to chromosomal DNA adjacent to a specific sequence of interest and (2) DNA which repairs the specific sequence of interest upon recombination between the targeting DNA and the chromosomal DNA, and (b) a second vector (RNA or DNA) comprising a nucleic acid encoding a HEase which cleaves the HEase recognition site present in the first vector. In a second embodiment, the method comprises introducing to the individual (a) a vector comprising targeting DNA, wherein the targeting DNA is flanked by a HEase recognition site and comprises (1) DNA homologous to chromosomal DNA adjacent to a specific sequence of interest (2) DNA which repairs the specific sequence of interest upon recombination between the targeting DNA and the chromosomal DNA, and (b) a HEase which cleaves the HEase recognition site present in the vector. In a third embodiment, the method comprises introducing to the individual a vector comprising (a) targeting DNA, wherein the targeting DNA is flanked by a HEase recognition site and comprises (1) DNA homologous to chromosomal DNA adjacent to a specific sequence of interest and (2) DNA which repairs the specific sequence of interest upon recombination between the targeting DNA and the chromosomal DNA, and (b) nucleic acid encoding a HEase which cleaves the HEase recognition site present in the plasmid. Preferably, the targeting DNA is flanked by two HEase recognition sites. Typically, the homologous DNA of the targeting DNA construct flanks each end of the DNA which repairs the specific sequence of interest. That is, the homologous DNA is at the left and right arms of the targeting DNA construct and the DNA which repairs the sequence of interest is located between the two arms. The vectors may be introduced to the individual in a cell or other suitable delivery mechanism.

The disclosure also relates to the generation of animal models of disease in which HEase recognition sites are introduced at the site of the disease gene for evaluation of optimal delivery techniques.

The efficiency of gene modification/repair may be enhanced by the addition expression of other gene products. The restriction endonuclease and other gene products may be directly introduced into a cell in conjunction with the correcting DNA or via RNA expression.

The present disclosure provides, in part, a method of cleaving a target nucleic acid comprising the homing endonuclease recognition sequence set forth in SEQ ID NO: 21, the method comprising providing a cell comprising:
a. a target nucleic acid comprising said homing endonuclease recognition sequence, and
b. a polypeptide comprising the sequence set forth in SEQ ID NO: 1, whereby the polypeptide cleaves the target nucleic acid.

The present disclosure provides, in part, a method of cleaving a target nucleic acid comprising the homing endonuclease recognition sequence set forth in SEQ ID NO: 22, the method comprising providing a cell comprising:
a. a target nucleic acid comprising said homing endonuclease recognition sequence, and
b. a polypeptide comprising the sequence set forth in SEQ ID NO: 13, whereby the polypeptide cleaves the target nucleic acid.

The present methods may be performed within a prokaryotic cell.

The present disclosure provides, in part, a method for site-directed homologous recombination in a cell, comprising:
a. providing a cell comprising:
   i. a first nucleic acid; and
   ii. a target nucleic acid comprising the homing endonuclease recognition sequence set forth in SEQ ID NO:21 or SEQ ID NO:22, wherein the first nucleic acid and target nucleic acid comprise one or more homologous sequences, and
b. cleaving the target nucleic acid according to the present method whereby homologous recombination occurs between the one or more homologous sequences of the first nucleic acid and the target nucleic acid.

In the present method the first nucleic acid may be, for example, a plasmid and the target nucleic acid is within a plasmid. In an alternative, the first nucleic acid may be a plasmid and the target nucleic acid is within a chromosome of the host cell. In an alternative, the first nucleic acid and the target nucleic acid may be within a chromosome of the host cell.

The present disclosure provides, in part, a method of inserting a nucleic acid into a target nucleic acid the method comprising:
a. providing a host cell comprising:
   i. a first nucleic acid comprising a second nucleic acid to be inserted into a target nucleic acid; and
   ii. a target nucleic acid comprising the endonuclease recognition sequence set forth in SEQ ID NO:21 or SEQ ID NO:22, wherein the first nucleic acid and the target nucleic acid comprise one or more homologous sequences, and wherein the second nucleic acid is proximal to at least one of the one or more homologous sequences of the first nucleic acid; and
b. inducing site-directed homologous recombination between the first nucleic acid and the target nucleic acid according to the present method, whereby the second nucleic acid is inserted into the target nucleic acid.

In the present method the second nucleic acid may, for example, encode a polypeptide.

The present disclosure provides, in part, a method of deleting a nucleic acid from a target nucleic acid the method comprising:
a. providing a host cell comprising:
   i. a first nucleic acid; and
   ii. a target nucleic acid comprising a second nucleic acid proximal to the endonuclease recognition sequence of SEQ ID NO:21 or SEQ ID NO:22, wherein the first nucleic acid and the target nucleic acid comprise one or more homologous sequences, and wherein the second nucleic acid is proximal to the one or more homologous sequences of the target nucleic acid; and
b. inducing site-directed homologous recombination between the first nucleic acid and the target nucleic acid according to the present methods, whereby the second nucleic acid is deleted from the target nucleic acid.

The second nucleic acid may, for example, encode a polypeptide.

The present disclosure provides, in part, a host cell wherein the genome of said host cell has been modified to comprise a homing endonuclease recognition site. The host cell may for example be a bacteria.

A list of sequence identification numbers of the present disclosure is given in Table 1.

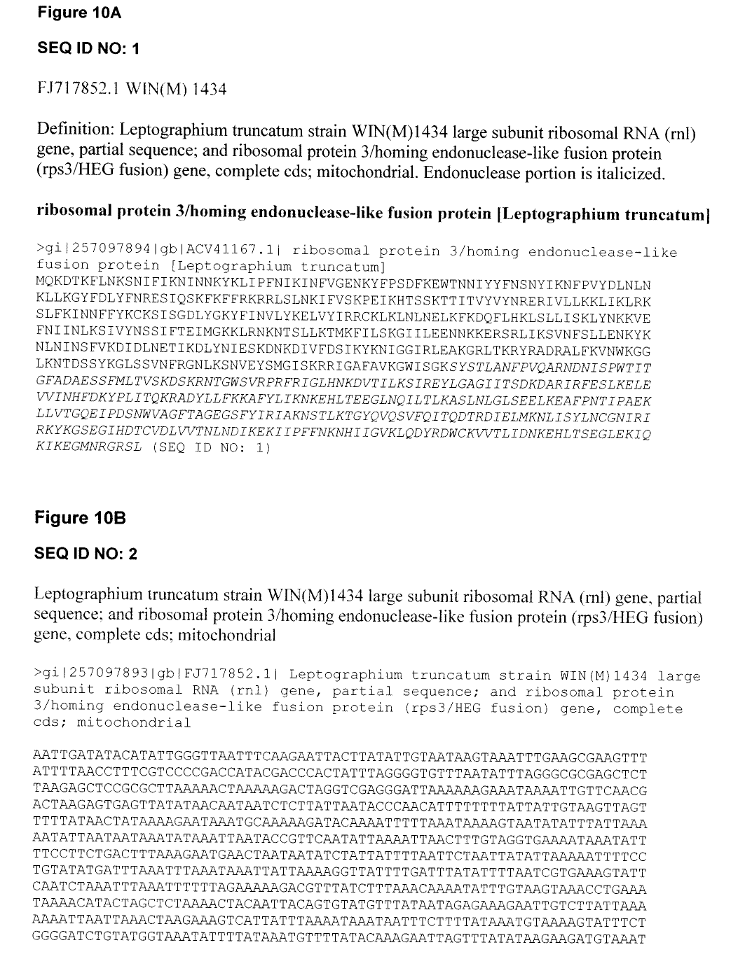

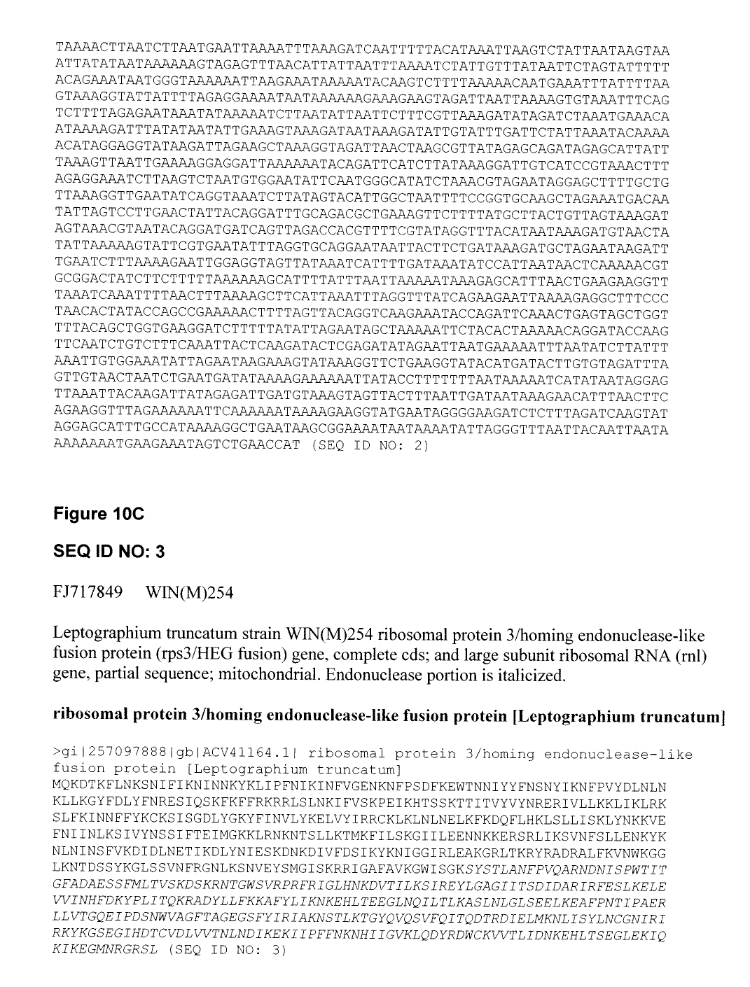

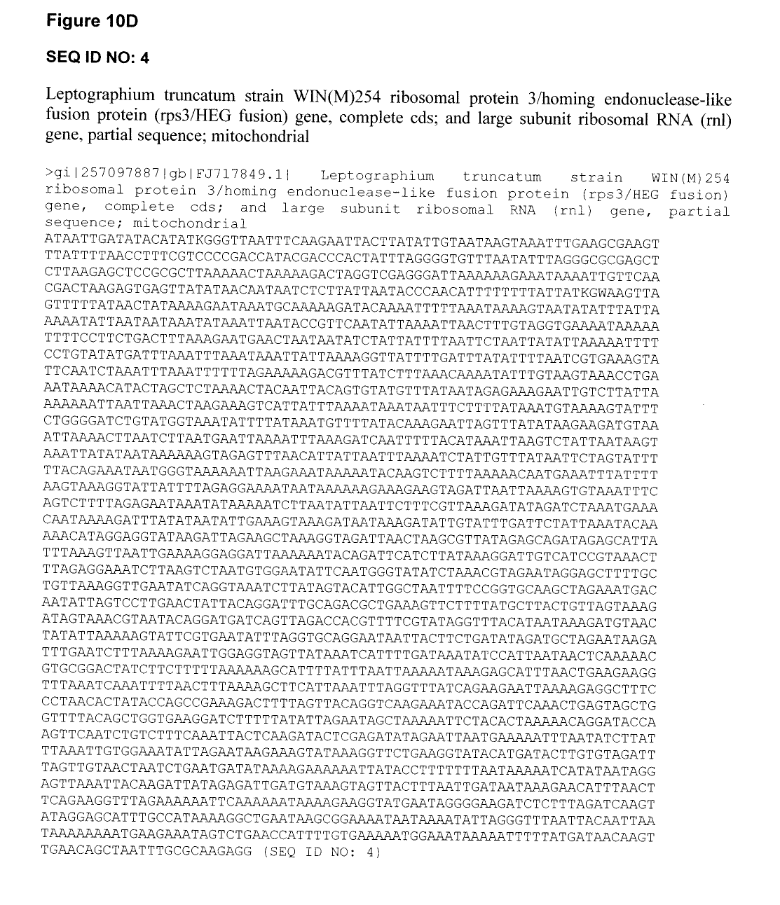

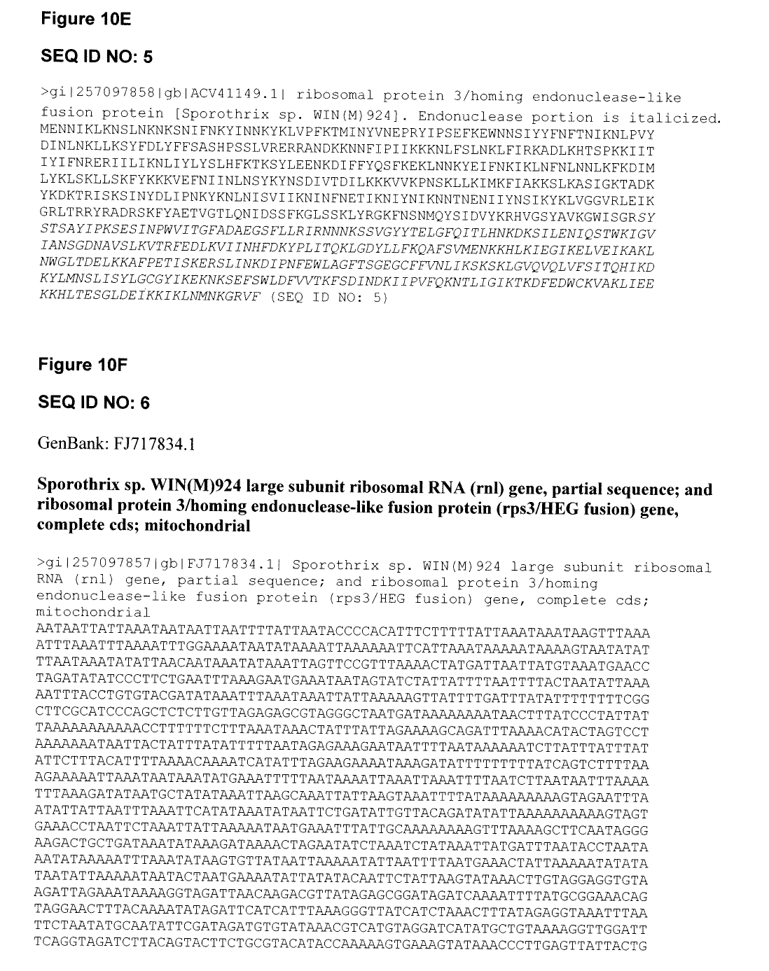

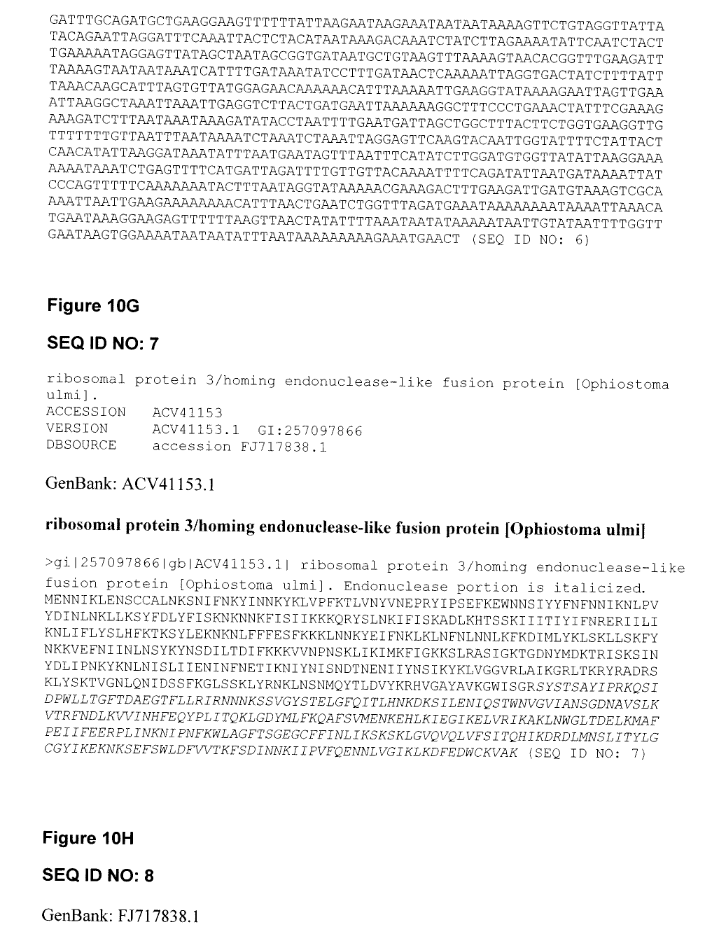

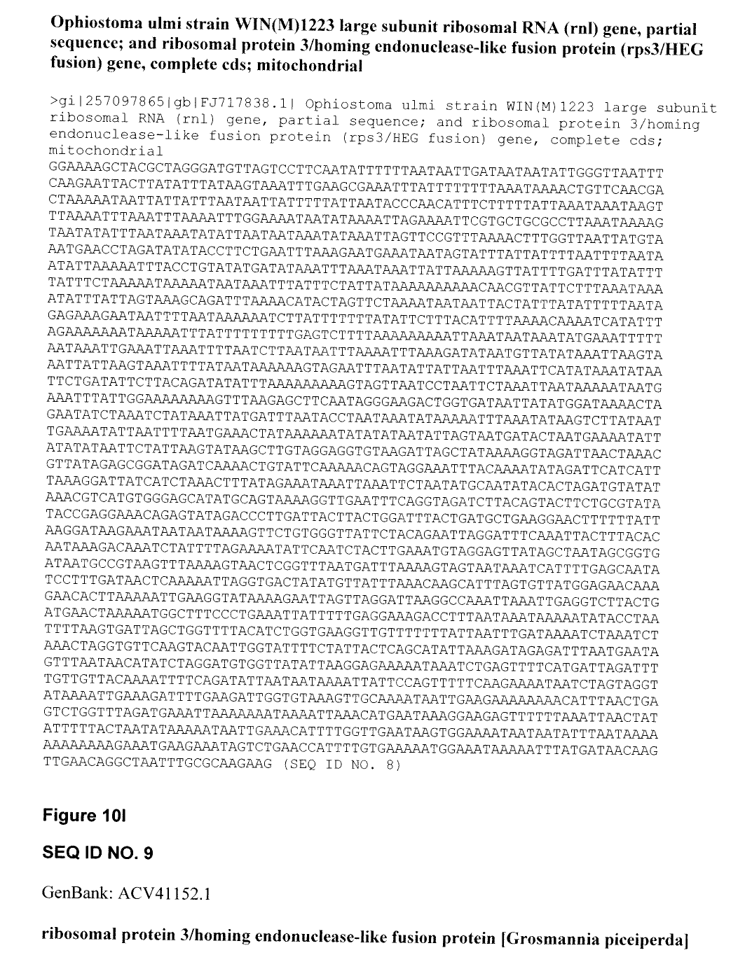

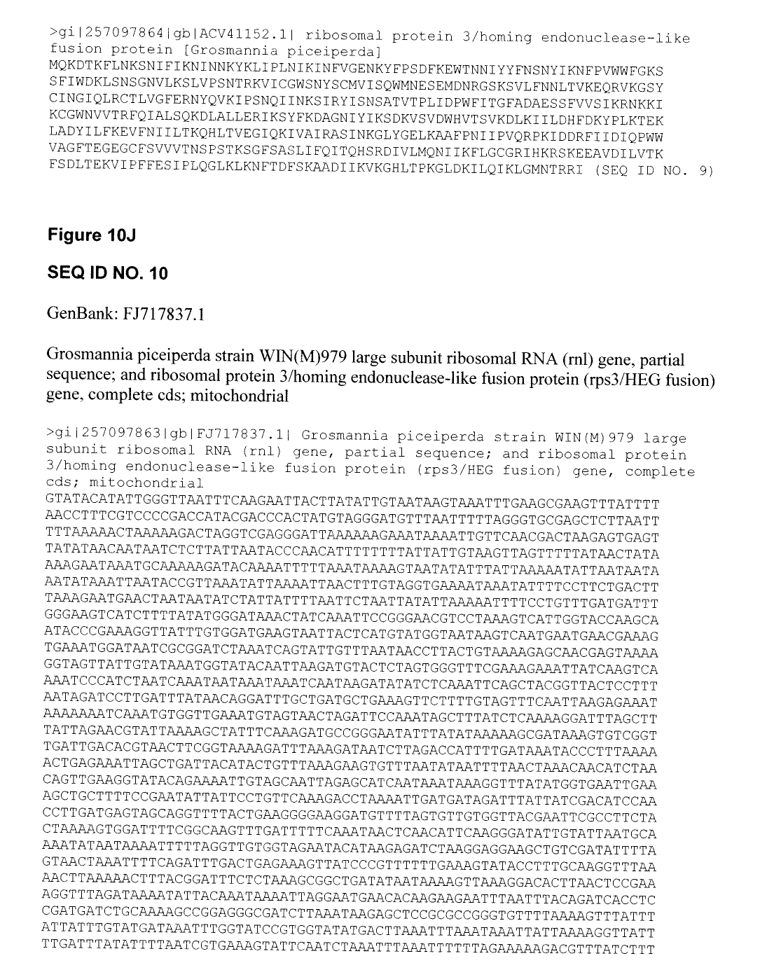

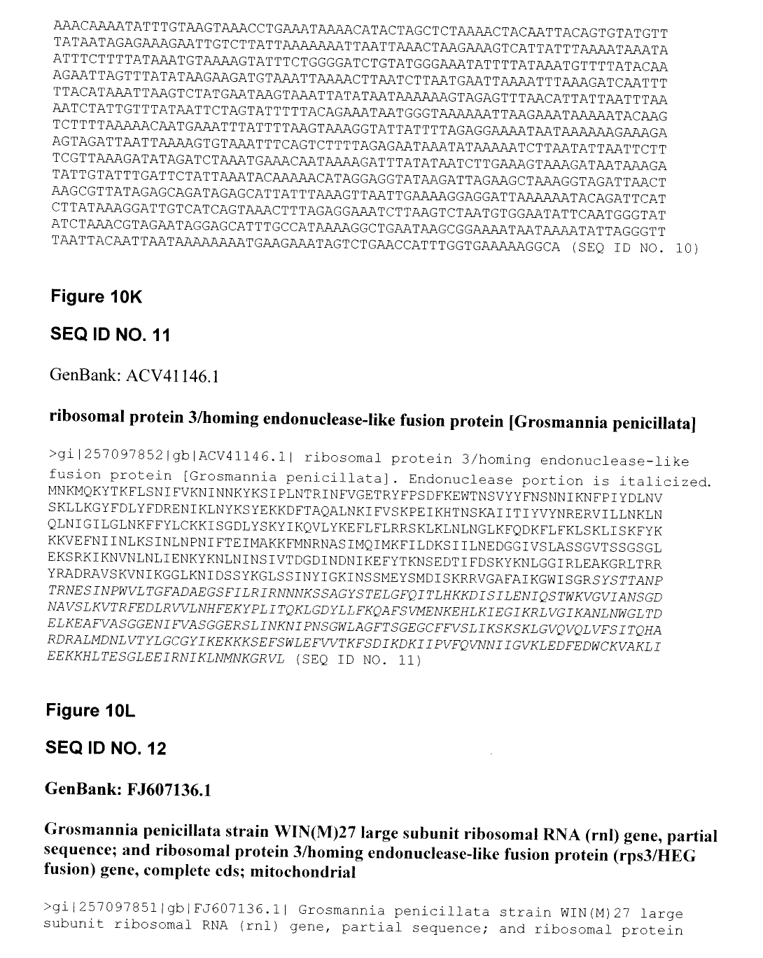

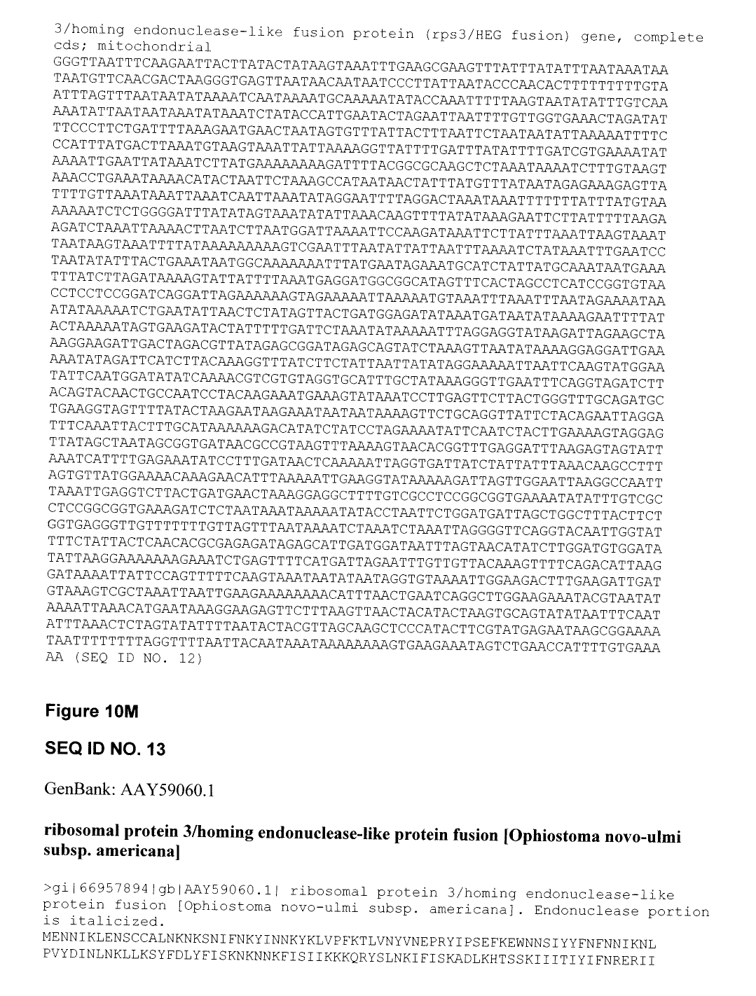

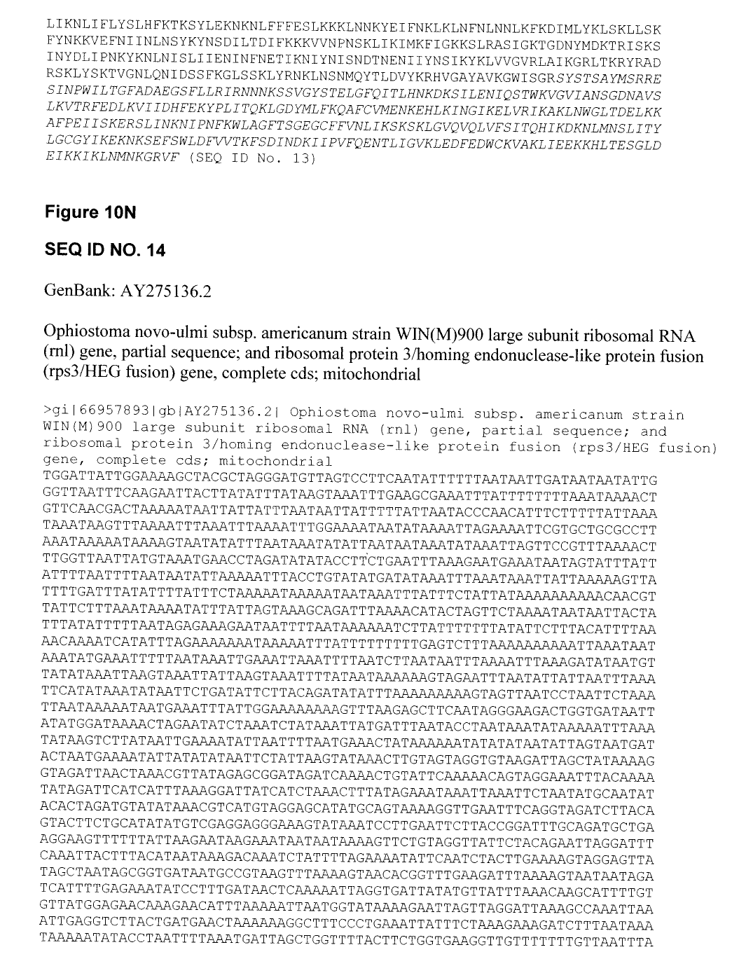

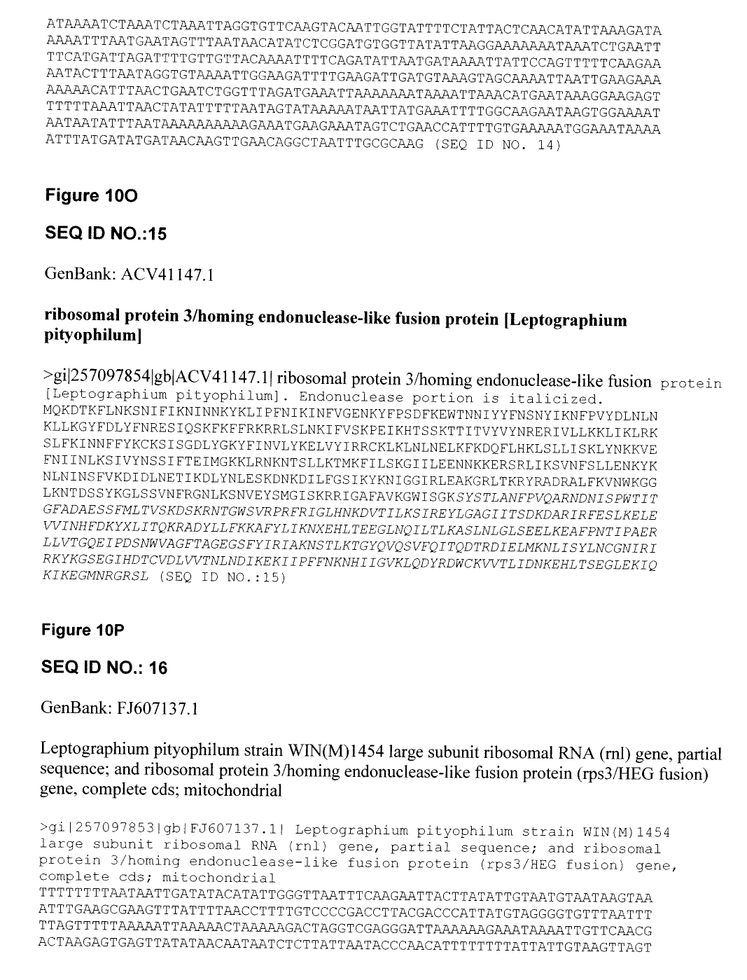

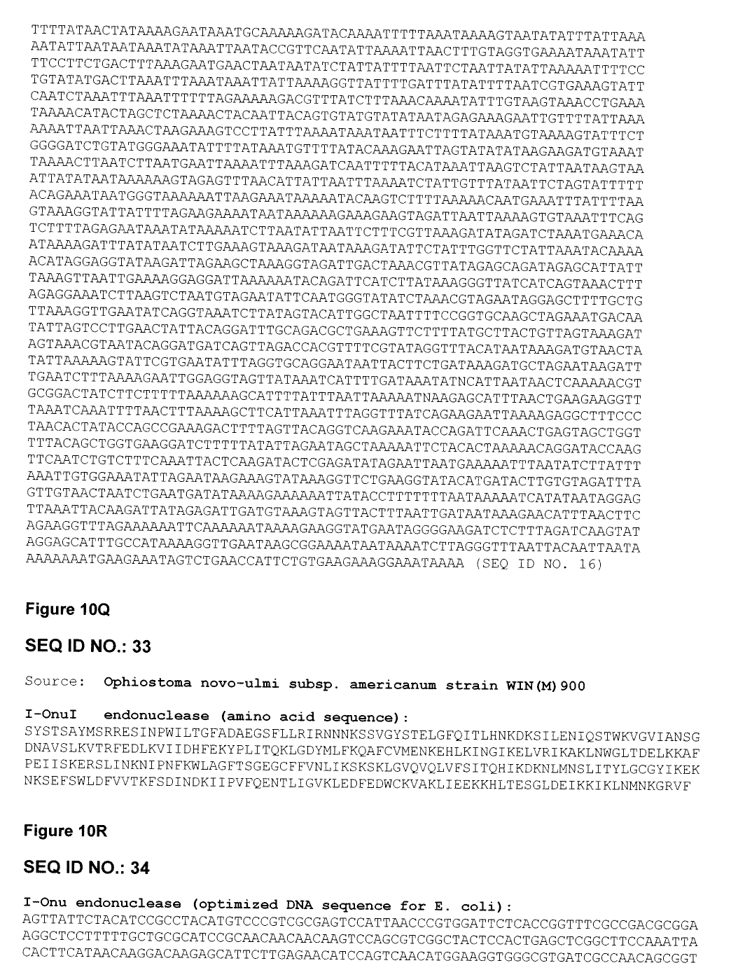

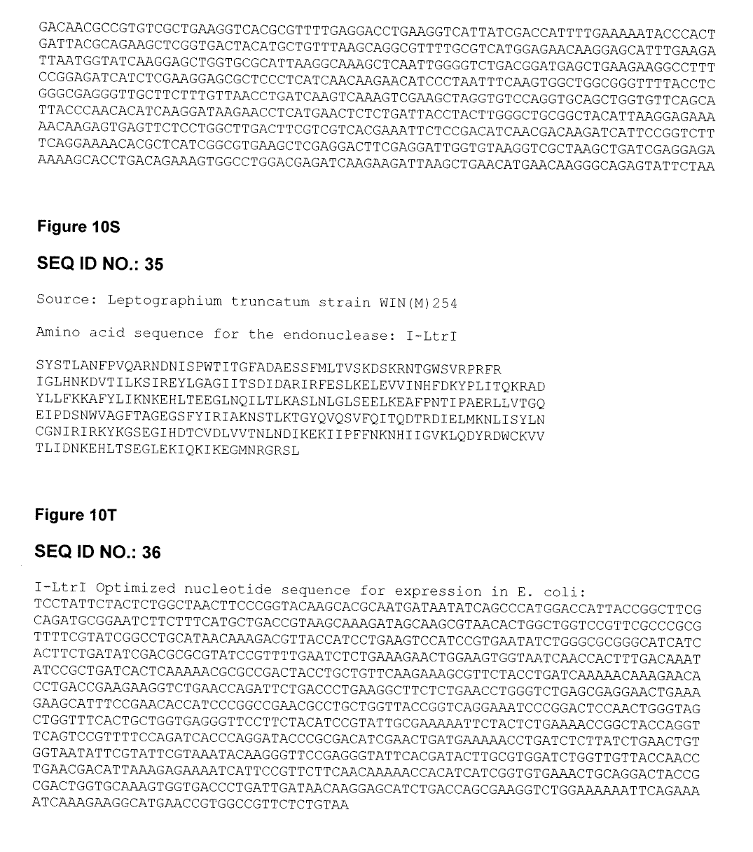

TABLE 1
List of Sequence Identification numbers (aa =
amino acid sequence; nt = nucleotide sequence}

| SEQ | | |
|---|---|---|
| ID | | Table/Figure |
| NO: | Description | or sequence |
| 1 | aa sequence of HEase | FIG. 10a |
| | (I-Ltr I) of Lepto- | |
| | graphium truncatum | |
| | (WIN M) 1434 | |
| 2 | nt sequence of HEase | FIG. 10b |
| | (I-Ltr I) Lepto- | |
| | graphium truncatum | |
| | (WIN M) 1434 | |
| 3 | aa sequence of HEase | FIG. 10c |
| | (I-Ltr-I) Lepto- | |
| | graphium truncatum | |
| | strain WIN(M)254 | |
| 4 | nt sequence of HEase | FIG. 10d |
| | HEase (I-Ltr I) from | |
| | Leptographium truncatum | |
| | (WIN M) 254 | |
| 5 | aa sequence of HEase | FIG. 10e |
| | from Sporothrix sp. | |
| | (WIN (M) 924) | |
| 6 | nt sequence of HEase | FIG. 10f |
| | from Sporothrix sp. | |
| | (WIN (M) 924) | |
| 7 | aa sequence of HEase | FIG. 10g |
| | from Ophiostoma ulmi | |
| | (WIN (M) 1223) | |
| 8 | nt sequence of HEase | FIG. 10h |
| | from Ophiostoma ulmi | |
| | (WIN (M) 1223) | |
| 9 | aa sequence of HEase | Fig. 10i |
| | from Grosmannia picei- | |
| | perda (WIN (M)(979) | |
| 10 | nt sequence of HEase | FIG. 10j |
| | from Grosmannia picei- | |
| | perda (WIN (M)(979) | |
| 11 | aa sequence of HEase | FIG. 10k |
| | from Grosmannia peni- | |
| | cillata (WIN (M)27) | |
| 12 | nt sequence of HEase | FIG. 10l |
| | from Grosmannia peni- | |
| | cillata (WIN (M)27) | |
| 13 | aa sequence of HEase | FIG. 10m |
| | (I-OnuI) from Ophio- | |
| | stoma novo-ulmi subsp. | |
| | Americanum (WIN (M)900) | |
| 14 | nt sequence of HEase | FIG. 10n |
| | (I-OnuI) from Ophio- | |
| | stoma novo-ulmi subsp. | |
| | Americanum (WIN (M)900) | |
| 15 | aa sequence of HEase | FIG. 10o |
| | from Leptographium | |
| | pityophilum WIN(M)1454 | |
| 16 | nt sequence of HEase | FIG. 10p |
| | from Leptographium | |
| | pityophilum WIN(M)1454 | |
| 17 | A-type consensus | AATTTTCCTGTATATGAC |
| 18 | B-type consensus | TCTAAACGTN₁GTATAGGAGCN |
| | | NNN |
| 19 | C-type consensus | AGGN₁TGN₂N₃TGAATAAGTGGA |
| 20 | C′-type consensus | TAAAAGGTTGAATAAN₁TGGA |
| 21 | I-LtrI recognition site | TCTAAACGTCGTATAGGAGCAT |
| | | TT |
| 22 | I-OnuI recognition site | GGTTGAATAAGTGG |
| 23 | Lsex-2R | CCTTGGCCGTTAAATGCGGTC |
| 24 | Lsex2-R-RT | TAGACGAGAAGACCCTATGCAG |
| 25 | IP2 | CTTGCGCAAATTAGC |
| 26 | LSEX-1 | GCTAGTAGAGAATACGAAGGC |
| 27 | LSEX-2 | GACCGCATTTAACGGCCAAGG |
| 28 | 900FP1 | AAATTAAATTCTAATATGC |
| 29 | 254synclmap1: | AAAGATAATAAAGATATTGTAT |
| | | TTG |
| 30 | exon-exon junction | CGCTAGGGAT/AACAGGCTAA |
| 31 | I-LtrI recognition site | AAATGCTCCTATACGACGTTTA |
| | complement strand | GA |
| 32 | I-OnuI recognition site | CCACTTATTCAACC |
| | complement strand | |
| 33 | aa sequence for endo- | 10Q |
| | nuclease (I-OnuI) from | |
| | Ophiostoma novo-ulmi | |
| | subsp. americanum | |
| | strain WIN(M)900 | |
| 34 | nt sequence for I-Onu | 10R |
| | endonuclease (optimized | |
| | DNA sequence for E. | |
| | coli): | |
| 35 | aa sequence for the | 10S |
| | endonuclease (I-LtrI) | |
| | from Leptographium | |
| | truncatum strain | |
| | WIN(M)254 | |
| 36 | nt sequence for I-LtrI | 10T |
| | Optimized nucleotide | |
| | sequence for expression | |
| | in E. coli: | |

The present invention will be further illustrated in the following examples. However it is to be understood that these examples are for illustrative purposes only, and should not be used to limit the scope of the present invention in any manner.

### EXAMPLES

### Example 1

### Identification of HEG Insertions

### Source and Maintenance of Fungal Cultures and DNA Extraction Protocols

Strains used in this study were from previous rDNA phylogenetic studies (Hausner et al. 1993, 2000; Hausner and Reid 2003). The sources for all strains used in this study are listed in table 1 S. All strains were cultured in petri dishes containing 2% malt extract agar (20 g malt extract [Difco, Michigan] supplemented with 1 g yeast extract [YE; Gibco, Paisly, United Kingdom] and 20-g bacteriological agar [Gibco] per liter). From these cultures, agar plugs were removed and used to inoculate 125-ml flasks containing 50 ml of PYG liquid medium (1 g peptone, 1 g YE, and 3 g glucose per liter) to generate biomass for DNA or RNA extraction (Hausner et al. 1992). The liquid cultures were still grown at 20 degree C. for up to 5 days and then harvested onto Whatman #1 filter paper via vacuum filtration. The harvested mycelium was homogenized by vortexing in the presence of 4 ml (volume) of small glass beads (equal ratio of 0.5- and 3-mm beads) in 6 ml of extraction buffer (10 mM Tris-HCl pH7.6, 1 mM ethylenediaminetetraacetic acid [EDTA], 50 mM NaCl, 1% hexadecyl trimethyl ammoniumbromide, and 0.5% sodium dodecyl sulfate [SDS]) and then incubated at 60 degree C. for 2 h. The lysate was mixed with an equal volume of chloroform and centrifuged at 2,000×g. About 5 ml of aqueous layer was recovered and mixed with 12 ml of ice cold 95% ethanol. The precipitated DNA was centrifuged for 30 min at 3,000×g, and the resulting pellet resuspended in 400 μl Tris-EDTA buffer (Tris-HCl, 1.0 mM EDTA, pH 7.6).

TABLE 1S
List of strains survey for the presence or absence of HEG insertions within the mL2449 intron
encoded RPS3 gene. Note that “S” indicates the absence of a HEG insertion whereas “L” suggests
the presence of an insertion within the mL2449 encoded RPS3 gene.

| Organism | Strain number | Product size (short or long) |
|---|---|---|
| Beauveria brongniartii | CBS¹ 128.53 | S |
| Ceratocystiopsis minuta | WIN(M)459 | S |
| Ceratocystiopsis minuta-bicolor | WIN² (M)479 | S |
| Ceratocystiopsis minuta-bicolor | WIN(M)480 | S |
| Ceratocystiopsis brevicomi | WIN(M)1452 | L |
| Ceratocystiopsis collifera | CBS 126.89 | S |
| Ceratocystiopsis concentrica | WIN(M)71-07 | S |
| Ceratocystiopsis minima | WIN(M)61 | S |
| Ceratocystiopsis minuta-bicolor | WIN(M)480 | S |
| Ceratocystiopsis minuta-bicolor | WIN(M)479 | S |
| Ceratocystiopsis pallidobrunnea | WIN(M)51(=69-14) | S |
| Ceratocystiopsis parva | WIN(M)59 | S |
| Ceratocystiopsis ranaculosus | WIN(M)919 | S |
| Ceratocystis coerulescens | WIN(M)98 | S |
| Ceratocystis coerulescens | WIN(M)931 | S |
| Ceratocystis coerulescens-resiniffera | WIN(M)79 | S |
| Ceratocystis curvicollis^{#7} | WIN(M)55(=70-25) | L |
| Ceratocystis deltoideospord^{#} | WIN(M)4 1(=71-26) | S |
| Ceratocystis deltoideospora^{#} | CBS 187.86 | S |
| Ceratocystis eucastaneae^{#} | WIN(M)512 | S |
| Ceratocystis eucastaneae^{#} | CBS 424.77 | S |
| Ceratocystis fagacearum | ATCC³ 24789 | S |
| Ceratocystis fimbriata | DAOM⁴ 195303 | S |
| Ceratocystis moniliformis | CBS 773.77 | S |
| Ceratocystis ossiformis^{#} | WIN(M)52 | S |
| Ceratocystis radicicola | CBS 114.47 | S |
| Ceratocystis tubicolfis^{#} | WIN(M)57 | S |
| Cornuvesica falcata | UAMH⁵ 9702 | S |
| Cornuvesica falcata | WIN(M)793 | S |
| Cornuvesica falcata | WIN(M)446 | S |
| Gabarnaudia betae | CBS 350.70 | S |
| Gelasinospora tetrasperma | ATTC 11345 | S |
| Gondwanamyces proteae | CBS 486.88 | S |
| Kernia pachypleura | WIN(M)253 | S |
| Leptographium pithyophilum | WIN(M)1454 | L |
| Leptographium procerum | WIN(M)1250 | S |
| Leptographium truncatum | WIN(M)1434 | L |
| Leptographium truncatum | WIN(M)254 | L |
| Leptographium truncatum | WIN(M)1435 | S |
| Neosartotya fischeri | CBS 525.65 | S |
| Ophiostoma narcissi | WIN(M)511 | S |
| Ophiostoma abietinum | CBS 125.89 | S |
| Ophiostoma abietinum | WIN(M)886 | S |
| Ophiostoma adjunctum | ATCC 34942 | S |
| Ophiostoma albidum | WIN(M)60-15 | S |
| Ophiostoma albidum | WIN(M)B-23 | S |
| Ophiostoma aureum | CBS 438.69 | S |
| Ophiostoma bicolor | ATCC 62329 | S |
| Ophiostoma bicolor | ATCC 15007 | S |
| Ophiostoma brunneo-ciliatum | WIN(M)89(=B-24) | S |
| Ophiostoma brunneum | CBS 161.11 | S |
| Ophiostoma canum | WIN(M)31 | S |
| Ophiostoma coronatum | WIN(M)867 | S |
| Ophiostoma coronatum | WIN(M)868 | S |
| Ophiostoma crassivaginata | WIN(M)1589 | S |
| Ophiostoma crenulatum | WIN(M)58 | S |
| Ophiostoma cucullatum | WIN(M)447 | S |
| Ophiostoma distortum | ATCC 22061 | S |
| Ophiostoma dryocetidis | CBS 376.66 | S |
| Ophiostoma europhioides | WIN(M)1430 | L |
| Ophiostoma europhioides | WIN(M)1431 | L |
| Ophiostoma europhioides | WIN(M)449 | L |
| Ophiostoma flexuosum | NFRI⁶ 81-79/10 | S |
| Ophiostoma francke-grosmanniae | ATCC22061 | S |
| Ophiostoma grande | CBS 350.78 | S |
| Ophiostoma himal-ulmi | CBS 374.67 | L |
| Ophiostoma huntii | WIN(M)492 | S |
| Ophiostoma hyalothecium | ATTC 28825 | S |
| Ophiostoma introcitrinum | WIN(M)69-47 | S |
| Ophiostoma ips | WIN(M)88-141 | L |
| Ophiostoma ips | WIN(M)88-105 | L |
| Ophiostoma ips | WIN(M)839 | L |
| Ophiostoma ips | WIN(M)83d | L |
| Ophiostoma ips | WIN(M)182 | L |
| Ophiostoma ips | WIN(M)92 | L |
| Ophiostoma ips | WIN(M)923 | L |
| Ophiostoma ips | WIN(M)1487 | S |
| Ophiostoma laricis | WIN(M)1461 | L |
| Ophiostoma longirostellatum | CBS 134.51 | S |
| Ophiostoma longisporum | WIN(M)48 | S |
| Ophiostoma manitobense | WIN(M)237 | S |
| Ophiostoma megalobrunneum | WIN(M)509 | L |
| Ophiostoma microsporum | CBS 412.77 | S |
| Ophiostoma minus | WIN(M)888 | S |
| Ophiostoma minus | WIN(M)861 | L |
| Ophiostoma montium | WIN(M)887 | S |
| Ophiostoma montium | CBS 151.78 | S |
| Ophiostoma montium | ATCC24285 | S |
| Ophiostoma montium | WIN(M)503 | S |
| Ophiostoma montium | WIN(M)495 | S |
| Ophiostoma montium | WIN(M)497 | S |
| Ophiostoma nigrum | CBS 163.61 | S |
| Ophiostoma olivaceum | CBS 138.51 | S |
| Ophiostoma penicillatum | WIN(M)27 | L |
| Ophiostoma penicillatum | WIN(M)165 | S |
| Ophiostoma penicillatum | WIN(M)448 | S |
| Ophiostoma penicillatum | CBS 212.67 | S |
| Ophiostoma penicillatum | WIN(M)136 | S |
| Ophiostoma piceaperdum | WIN(M)979 | L |
| Ophiostoma piliferum | WIN(M)973 | S |
| Ophiostoma pluriannulatum | CBS 434.77 | S |
| Ophiostoma polyporicola | CBS 669.88 | S |
| Ophiostoma populinum | CBS 212.67 | S |
| Ophiostoma pseudoeurophioides | WIN(M)42 | S |
| Ophiostoma pseudonigrum | W IN(M)71-13 | S |
| Ophiostoma rolhansenianum | WIN(M)110 | S |
| Ophiostoma rolhansenianum | WIN(M)113 | S |
| Ophiostoma rostrocoronatum | CBS 434.77 | S |
| Ophiostoma seticollis | CBS 634.66 | S |
| Ophiostoma sparsum | CBS 405.77 | S |
| Ophiostoma stenoceras | CBS 237.32 | S |
| Ophiostoma tremoloaureum | CBS 361.65 | S |
| Ophiostoma tetropii | WIN(M)111 | L |
| Ophiostoma tetropii | WIN(M)451 | L |
| Ophiostoma torulosum | WIN(M)730 | L |
| Ophiostoma ulmi⁸ | WIN(M)1223 | L |
| Ophiostoma vesicum | CBS800.73 | S |
| Sordaria fimicola | ATCC 6739 | S |
| Sphaeronaemella fimicola | UAMH 8839 | S |
| Sphaeronaemella fimicola | WIN(M)818 | S |
| Sporothrix sp. | WIN(M)924 | L |

¹CBS = Centraal Bureau voor Schimmelcultures, Utrecht, The Netherlands;
²WIN(M) = University of Manitoba (Winnipeg) Collection;
³ATCC = American Type Culture Collection, Manassas,VA, USA;
⁴DAOM = Canadian National Mycological Herbarium, Ottawa, ON, Canada;
⁵UAMH = University of Alberta Microfungus Collection & Herbarium, Devonian Botanic Garden, Edmonton, AB, Canada;
⁶NFRI = Norwegian Forest Research Institute, As, Norway;
⁷#denotes species that should be transferred to Ophiostoma;
⁸note additional 21strains of O. ulmi and 197 strains O. novo-ulmi subsp. americana have been previously screened by Gibb and Hausner (2005) and Sethuraman et al. (2008).

### Polymerase Chain Reaction (PCR) Amplification, Cloning of PCR Products, and DNA Sequencing

A PCR-based survey utilizing primers primers IP1 (GGAAAAGCTACGCTAGGG) and IP2 (CTTGCGCAAATTAGCC) (Bell et al. 1996) was conducted in order to examine the mt-rnl U11 intron in members of Ophiostoma and related taxa for the presence of potential HEG insertions. Between 50 and 100 ng of whole-cell DNA served as a template for PCR reactions. Taq polymerase, buffers, and deoxyribonucleotide triphosphates were obtained from Invitrogen (Life Technologies, Burlington, ON) and used according to the manufacturer's recommendations. Typically, PCR conditions were as follows: an initial denaturation step of 94 degree C. for 3 min was followed by 25 cycles of denaturing (93 degree C. for 1 min), annealing (52.9 degree C. for 1 min 30 s) and extension (70 degree C. for 4 min 30 s) followed by cooling the reactions to 4 degree C. PCR fragments were separated by gel electrophoresis through a 1% agarose gel in Tris-borate-EDTA buffer (89 mM Tris-borate buffer with 10 mM EDTA at pH 8.0). DNA fragments were sized using the 1-kb-plus DNA ladder (Invitrogen) and the DNA fragments were visualized by staining with ethidium bromide (0.5 pg/ml).

PCR products were used directly as templates for DNA sequence analysis or products cloned using the Topo TA cloning kit (Invitrogen). The PCR products were purified with the Wizard SV Gel and PCR clean-up system (Promega), and plasmid DNA was purified using the Wizard Plus Minipreps DNA purification system (Promega). The sequencing reactions were performed at the University of Calgary Core DNA services facility (Calgary, AB). Table 2 lists the strains that were examined by DNA sequence analysis and also provides the GenBank accession for sequences obtained in this study. Initially, sequencing employed the IP1 and IP2 primers, or when appropriate for cloned PCR products, the M13 forward and reverse primers were used; thereafter, nested primers were designed as needed. DNA sequences were obtained for both strands. Oligonucleotides used in this study were synthesized by Alpha DNA (Montreal, Que, Canada).

Reverse Transcriptase-PCR (RT-PCR) Analysis for the rnl-U11 Segment

RNA was isolated from strain O. novo-ulmi subsp. americana WIN(M) 900 using the RNeasy kit for total RNA isolation (Qiagen Sciences, MD) with some modifications. Initially, the mycelium was ground in liquid nitrogen. However, once the cell walls were broken, the RNA was extracted and purified following the yeast protocol of the RNeasy kit. The RNA was treated with DNase (Ambion) following the manufacturer's recommendation, and 1 μg of RNA was used as template for RT-PCR using the ThermoScript RT-PCR system (Invitrogen) according to manufacture's recommendations. First-strand synthesis was carried out with primer IP2 at a final concentration of 10 μM and subsequent PCR amplification was carried out with primers Lsex-2R (CCTTGGCCGTTAAATGCGGTC—SEQ ID NO.: 23) and IP2 (10 μM concentration). The PCR products generated by the RT-PCR reaction were cloned into the Topo TA cloning kit (Invitrogen) and sequenced with primers Lsex2-R-RT (TAGACGAGAAGACCCTATGCAG—SEQ ID NO.: 24) and IP2 (CTTGCGCAAATTAGC—SEQ ID NO.: 25) (Bell et al. 1996).

### Sequence and Phylogenetic Analysis

The individual sequences were assembled manually into contigs using the GeneDoc program v2.5.010 (Nicholas et al. 1997). The ORF Finder program (http://www.ncbi.nlm.nih.gov/gorf/gorf.html) was used (setting: genetic code for mtDNA of molds) to search for potential ORFs within the ml-U11 group I introns. The online resource BlastP (Altschul et al. 1990) was used to retrieve sequences that were related to the putative ORFs obtained from our strains (table 2). Sequences were aligned and refined manually with the aid of the GeneDoc program. For phylogenetic analyses, only those segments of the alignment where all sequences could be aligned unambiguously were retained. Phylogenetic estimates were generated by the programs contained within the PHYLIP package (Felsenstein 1989, 2005) and the MrBayes program v3.1 (Ronquist and Huelsenbeck 2003; Ronquist 2004). In PHYLIP, a phylogenetic tree was obtained by analyzing the alignment with the PROTPARS (protein parsimony algorithm, version 3.55 c) program in combination with bootstrap analysis (SEQBOOT) and CONSENSE to obtain the majority rule consensus tree along with an estimate of confidence levels for the major nodes within the phylogenetic tree (Felsenstein 1985). Phylogenetic estimates were also generated within PHYLIP using the NEIGHBOR program using distance matrices generated by PROTDIST (setting: Dayhoff PAM250 substitution matrix; Dayhoff et al. 1978). The MrBayes program was used for Bayesian analysis. The amino acid substitution model setting for Bayesian analysis was as follows: mixed models and gamma distribution with four gamma rate parameters. The Bayesian inference of phylogenies was initiated from a random starting tree and four chains were run simultaneously for 1,000,000 generations; trees were sampled every 100 generations. The first 25% of trees generated were discarded (“burn-in”) and the remaining trees were used to compute the posterior probability values. Phylogenetic trees were drawn with the TreeView program (Page 1996) using PHYLIP tree outfiles or MrBayes tree files and annotated with Corel Draw (Corel Corporation and Corel Corporation Limited).

TABLE 2
List of Strains, Presence and Absence of RPS3 HEG Insertions, Category of HEG Insertion, and Genbank Accession Numbers

| | | Presence | Position | | Genbank |
|---|---|---|---|---|---|
| Organism | Strain Number | of HEGa | of HEG^{b} | Degeneratedc | Accession |
| Ceratocystiopsis brevicomi | WIN^{d}(M) 1452 | L | C | Yes^{e} | FJ717840 |
| Ceratocystis curvicollis (5 Ophiostoma | WIN(M) 55 | L | C | Yes | FJ717842 |
| nigrum sensu Upadhyay 1981) | | | | | |
| Ceratocystiopsis minuta-bicolor | WIN(M) 480 | S | | | FJ717855 |
| Ceratocystiopsis parva | WIN(M) 59 | S | | | FJ717754 |
| Ophiostoma aureum | CBS^{f} 438.69 | S | | | FJ717847 |
| Ophiostoma distortum | WIN(M) 847 (=ATCC^{g} 18998) | L | C | Yes | FJ717845 |
| Ophiostoma europhioides | WIN(M) 449 | L | B | Yes | FJ717841 |
| | WIN(M) 1430 | L | B | Yes | FJ717836 |
| | WIN(M) 1431 | L | B | Yes | FJ717839 |
| Ophiostom himal-ulmi | CBS 374.67 | L | C | Yes | F1717862 |
| Ophiostoma ips | WIN(M) 923 | L | C' | Yes | FJ717857 |
| | WIN(M 1487 | S | | | FJ717858 |
| Ophiostoma laricis | WIN(M) 1461 | L | A/B | Yes (A/B) | FJ717851 |
| Ophiostoma megalobrunneum | WIN(M) 509 | L | C | Yes | FJ717856 |
| Ophiostoma minus | WIN(M) 861 | L | C | Yes | FJ717860 |
| | WIN(M) 888 | S | | | FJ717859 |
| Ophiostoma nigrum | CBS 163.61 | S | | | FJ717846 |
| Ophiostom novo-ulmi subsp. americana | WIN(M) 900 | L | C | No | AY275136 |
| | WIN(M) 904 | S | | | AY275137 |
| Ophiostoma penicillatum | WIN(M) 27 | L | C | No | FJ607136 |
| | WIN(M) 136 | S | | | FJ607138 |
| Ophiostoma piceaperdum | WIN(M) 979 | L | A | No | FJ717837 |
| Ophiostoma pseudoeurophioides | WIN(M) 42 | S | | | FJ717848 |
| Ophiostoma rollhansenianum | WIN(M) 113 | S | | | FJ717853 |
| Ophiostoma tetropii | WIN(M) 111 (=NFRIh 80-113/9) | L | C | Yes | FJ717843 |
| | WIN(M) 451 | L | C | Yes | FJ717844 |
| Ophiostoma torulosum | WIN(M) 730 (=CBS 770.71) | L | C | Yes | FJ717861 |
| Ophiostoma ulmi | WIN(M) 1223 | L | C | No | FJ717838 |
| Leptographium lundbergii | WIN(M) 1250 | S | | | FJ717850 |
| Leptographium pithyophilum | WIN(M) 1454 | L | B | No | FJ607137 |
| Leptographium truncatum | WIN(M) 254 | L | B | No | FJ717852 |
| | WIN(M) 1434 | L | B | No | FJ717849 |
| | WIN(M) 1435 | S | | | FJ717835 |
| Sporothrix sp. | WIN(M) 924 | L | C | No | FJ717834 |

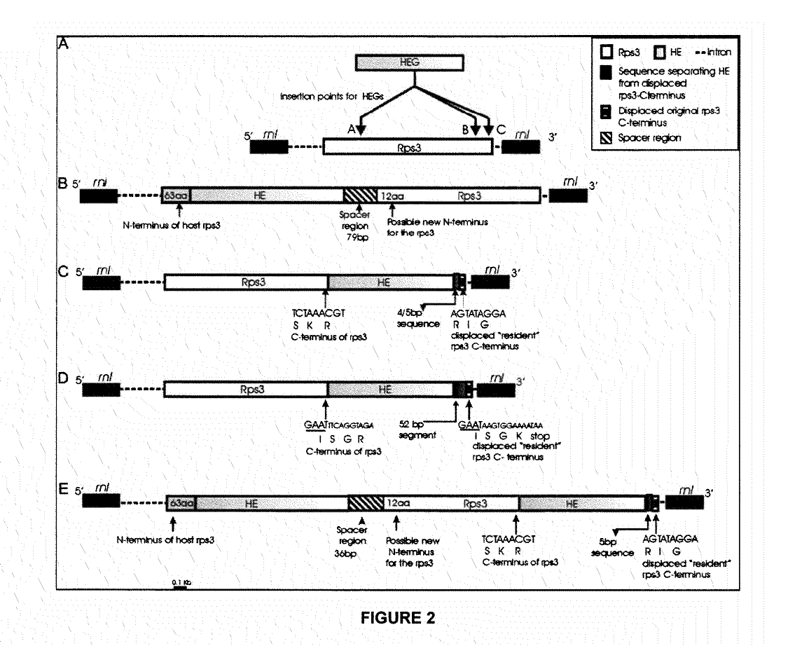

a“S” indicates the absence of an HEG insertion whereas “L” suggests the presence of an insertion within the mL2449 encoded RPS3 gene.
^{b}Positions based on A, B, and C designations in FIG. 2.
cPresence of frameshift mutations and premature stop codons are viewed as evidence for degeneration.
^{d}W1N(M) = University of Manitoba (Winnipeg) Collection.
^{e}Yes = HEase ORF is degenerated, No = HE ORF appears to be intact.
^{f}CBS = Centraal Bureau voor Schimmelcultures, Utrecht, The Netherlands.
^{g}ATCC = American Type Culture Collection, Manassas, VA.
hNFRI = Norwegian Forest Research Institute, As, Norway.

### Example 2

### Expression and Purification of HEase

### Expression and Purification of I-OnuI and I-LtrI

For expression of I-OnuI and I-LtrI in E. coli, codon modified versions of these genes were constructed synthetically, taking into account differences between the fungal mitochondrial and E. coli genetic code (BioS&T, Montreal, Que, Canada). Both the I-OnuI and I-LtrI genes were cloned into pBlueScript II SK+, and then subcloned into pTOPO-4 (Invitrogen). Subsequently, the I-OnuI and I-LtrI sequences were moved into pET200/D-TOPO (Invitrogen) with the N terminal His-tag intact to generate pI-OnuI and pI-LtrI, which were subsequently transformed into E. coli strain ER2566 (New England Biolabs, NEB) for expression studies.

To express and purify I-OnuI or I-LtrI, a 10-ml E. coli culture containing pI-OnuI or pI-LtrI was grown overnight and diluted 1:100 into 1 l of Luria-Bertani media. The 1 l culture was grown at 37 degree C. until A₆₀₀˜0.4, shifted to 27 degree C., and expression induced by adding isopropyl-β-D-thiogalactopyranoside to a final concentration of 1 mM. After additional growth for 2.5 h, cells were harvested by centrifugation at 5000 rpm for 5 min and the pellet was frozen at −80 degree C. For protein purification, the frozen cells were thawed in the presence of protease inhibitor (Roche Diagnostic) and resuspended in 10 ml of lysis buffer (20 mM Tris-HCl, pH 7.9, 500 mM NaCl, 40 mM imidazole and 10% glycerol) per 1 gm of wet cell weight. Cells were disrupted by homogenization followed by centrifugation at 27,200×g for 25 min at 4 degree C. The supernatant was sonicated to facilitate DNA fragmentation, and centrifuged at 20,400×g for 15 min at 4 degree C. The supernatant was applied to a HisTrap HP Affinity column (GE Healthcare) that had been charged with 0.1 M NiSO₄ and equilibrated with binding buffer (20 mM Tris-HCl, pH7.9, 500 mM NaCl, 40 mM imidazole, and 10% glycerol). Bound proteins were eluted with elution buffer (20 mM Tris-HCl pH7.9, 500 mM NaCl, and 10% glycerol) over a linear gradient of imidazole from 0.08 to 0.5 M, and 500-μl fractions were collected over 50 ml. To prevent precipitation, 500 μl of 2 M NaCl and 10 μl of 0.5M EDTA, pH 8.0, were added to peak fractions. The peak fraction was loaded directly onto a Superdex 75 gel-filtration column (GE Healthcare) equilibrated with lysis buffer without immidazole. Fractions were collected in 0.25-ml aliquots over 25 ml Peak-containing fractions were pooled and aliquoted and frozen at −80 degree C.

### Example 3

### Mapping and Characterization of HEase Recognition Sites

### Endonuclease Assays

In vitro cleavage assays were carried out with the I-OnuI protein using a variety of possible substrates: 1) The RPS3-HEG-minus sequence was PCR amplified from O. novo-ulmi subsp. americana strain WIN(M) 904 (Gibb and Hausner 2005) and inserted into a pTOPO-4 (Invitrogen) vector. This construct (pRPS3) provided the HEG minus target substrate for cleavage and mapping assays; 2) a complete RPS3-HEG fusion was synthetically constructed (BioS & T) and inserted into pET200/D-TOPO (Invitrogen) to create pRPS3/HEG. This construct served as the HEG-containing substrate for cleavage assays; and 3) the mt-rnl-U7 region was amplified from Ceratocystis polonica strain WIN(M) 1409 using primers LSEX-1 (GCTAGTAGAGAATACGAAGGC—SEQ ID NO.: 26) and LSEX-2 (GACCGCATTTAACGGCCAAGG—SEQ ID NO.: 27) (Sethuraman et al. 2008) and inserted into the TOPO-4 vector. This construct, pU71409, served as a negative control for the cleavage assay.

Cleavage assays were carried out by incubating 200 ng of plasmid substrate in a total volume of 20 μl containing 1 μl of O-OnuI (25 ng), 2 μl NEB Buffer #3 (100 mM NaCl, 50 mM Tris-HCl, pH 7.9, 10 mM MgCl2, and 1 mM dithiothreitol) and 17 μl of H₂O at 37 degree C. Aliquots were taken at 5-min intervals for 30 min and stopped by the addition of loading buffer and stop solution (0.1M Tris-HCl, pH7.8, 0.25M EDTA, 5% w/v SDS, 0.5 μl/ml proteinase K). Reactions were analyzed by agarose gel electrophoresis and fragments were visualized by staining with ethidium bromide (0.5 μl/ml).

### Cleavage Site Mapping for I-OnuI and I-LtrI

In order to determine the cleavage sites for I-OnuI and I-LtrI, PCR products that included the putative cleavage site located near the 3# end of the RPS3-coding sequence were amplified from pRPS3 with primers end labeled on the noncoding (top) or coding (bottom) strand. The substrate molecule for the I-OnuI assay was a 201-bp product amplified by using primers 900FP1 (AAATTAAATTCTAATATGC—SEQ ID NO.: 28) and IP2 (Bell et al. 1996). Primers were 5′-end labeled with OptiKinase (USB, Cleveland, Ohio) according to the manufacturer's protocols using [γ-³²P]ATP. The 201-bp amplicons were generated using either 900FP1 or IP2 5′-end-labeled primers; thus, substrates could be generated where either the coding or the noncoding strands were labeled. The end-labeled PCR products were incubated with 1 μl I-OnuI for 10 min at 37 degree C. in 20-μl reaction mixtures consisting of 5-μl substrate, and 1× NEB Buffer #3. The resulting cleavage products were resolved on a denaturing 6% polyacrylamide/urea gel (19:1 acrylamide:bis-acrylamide) and electrophoresed alongside the corresponding sequencing ladders obtained from pRPS3 using the endlabeled primers (900FP1 and 1P2) (USB Biologicals).

The substrate for the I-LtrI assay was an RPS3 PCR product derived from the HEG-minus strain of L. truncatum WIN(M)1435. The cleavage site mapping assay was performed as for I-OnuI, but the following primers were used for generating the cleavage substrate and corresponding DNA-sequencing ladders: 254synclmapl: AAAGATAATAAAGATATTGTAT TTG (SEQ ID NO.: 29) and IP2.

### Example 4

### Identification and Characterization of HEG Insertion Sites

The rnl-U11 Intron and a PCR-Based Survey for RPS3 HEG Insertions

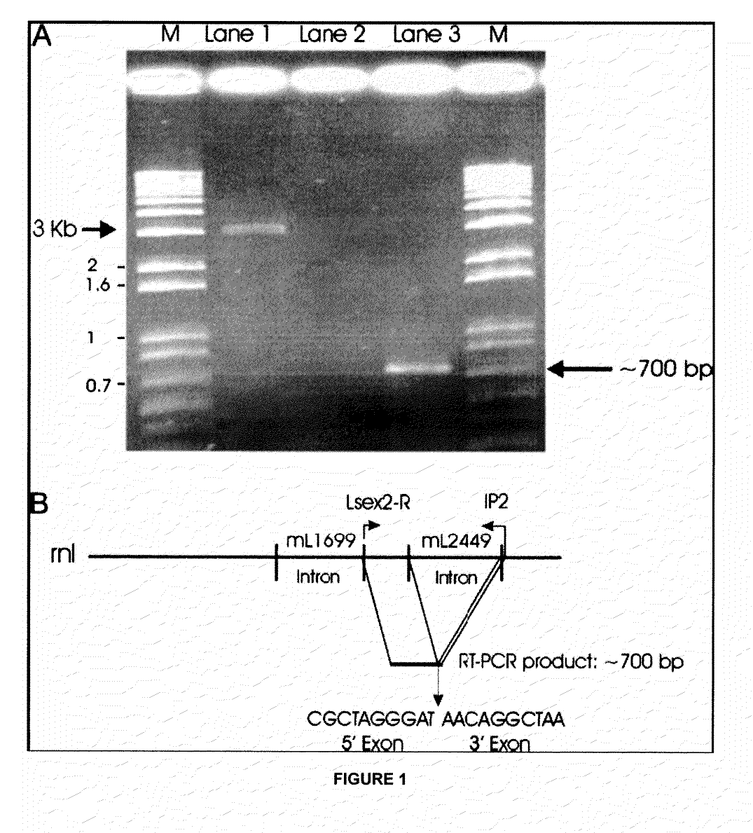

The rnl-U11 intron was previously characterized from a variety of filamentous ascomycetes such as P. anserina, C. parasitica, and O. novo-ulmi subsp. americana (reviewed in Hausner 2003; Gibb and Hausner 2005), and classified as a group I intron belonging to the IA1 subgroup based on sequence data and structural features. To confirm that this region indeed represents an intron, we performed RT-PCR on total RNA isolated from O. novo-ulmi subsp. americana strain WIN(M)900. Using primers that flank the intron insertion site, a 3-kb product was amplified from genomic DNA (FIG. 1, lane 1), whereas a 0.65-kb product was amplified from cDNA, the size expected to result from ligation of exons after intron splicing (FIG. 1, lane 3).We confirmed that the 0.65-kb product corresponded to ligated exons by cloning and sequencing the product, showing that the U 11 insertion is indeed an intron. Based on the sequence obtained from the RT-PCR product, the splice junction was as follows: 5′ exon-TAGGGAT/intron/AACAGG-3′exon. The intron insertion site corresponds to position L2449 of the E. coli LSU rDNA. To assess the diversity of HEG insertions within RPS3 genes that are encoded in the mL2449 group 1 intron, we performed a PCR-based survey with primers IP1 and IP2 that flank the mL2449 insertion site using total DNA isolated from 119 strains of ophiostomatoid fungi representing 85 species. Two categories of PCR products were amplified: short (1.6-kb) products for 88 strains, and long (2.4- to 3.0-kb) products for 31 strains (table 1S). Based on previous work on ophiostomatoid fungi and related taxa (Gibb and Hausner 2005; Sethuraman et al. 2008), we assumed that short PCR fragments most likely represented RPS3 genes within the L2449 intron that are not interrupted by a HEG (HEG-minus RPS3 alleles), whereas the long fragments represent RPS3 genes that are interrupted by a HEG (HEG-plus RPS3 alleles). We sequenced a total of 21 long PCR products to characterize the HEG insertions and also sequenced 11 short PCR products from closely related species to accurately localize the HEG insertion point. In summary, we identified three different HEG insertion sites within RPS3 alleles of ophiostomatoid fungi, all involving double-motif LAGLIDADG HEases (FIG. 2A). In addition to completely sequencing 21 of the long PCR products, we partially sequenced an additional 10 products, none of which revealed novel insertion sites/HEGs and were therefore not characterized any further. A-type HEG insertions were located in the N-terminal coding region of RPS3 (FIG. 2B), and B-type and C-type insertions were located within the C-terminal coding region of RPS3 (FIGS. 2C and D). The C-type insertions are similar to the insertion previously described for 0. novo-ulmi subsp. Americana (Gibb and Hausner 2005). In addition, we found one example where an A- and B-type HEG had independently inserted into a single RPS3 gene of Ophiostoma laricis (A/B-type insertion; FIG. 2E). Each of these insertions is described in detail below.

### A-Type HEG Insertions Create Bi-ORFic U11 ml Introns

Sequencing of the Ophiostoma piceaperdum strain IP PCR product resolved the size of the mL2449 intron to be 2.914 kb (FIG. 2B), whereas sequencing of a closely related species Ophiostoma aureum (CBS 438.69; Hausner et al. 1993) revealed a 1.6-kb mL2449 intron that lacked an HEG insertion in RPS3. This HEG-minus sequence was used as a reference to determine the insertion point of the HEG in the RPS3 gene of O. piceaperdum. The insertion of the LAGLIDADG HEG within the O. piceaperdum L2449 intron has created two putative ORFs. The first ORF is 1.446 kb, encoding a 482 amino acid fusion protein consisting of the first 189 by of RPS3 (the N-terminal 63 amino acids) followed by 1.257 kb (419 amino acids) that corresponds to a double-motif LAGLIDADG HEase. The second ORF within the O. piceaperdum U11 intron is separated from the first ORF by a 79-bp spacer region, is 1.041 kb long, and encodes a Rps3 homolog of 347 amino acids. The origin of 79-bp spacer sequence and the first 38-bp sequence of the second ORF (Rps3) in O. piceaperdum are unknown, as similar sequences are not found in the closely related O. aureum RPS3 sequence (or for that matter in any characterized rnl U11 sequence).

B- and C-Type Insertions Create Mono-ORFic mL2449 Introns

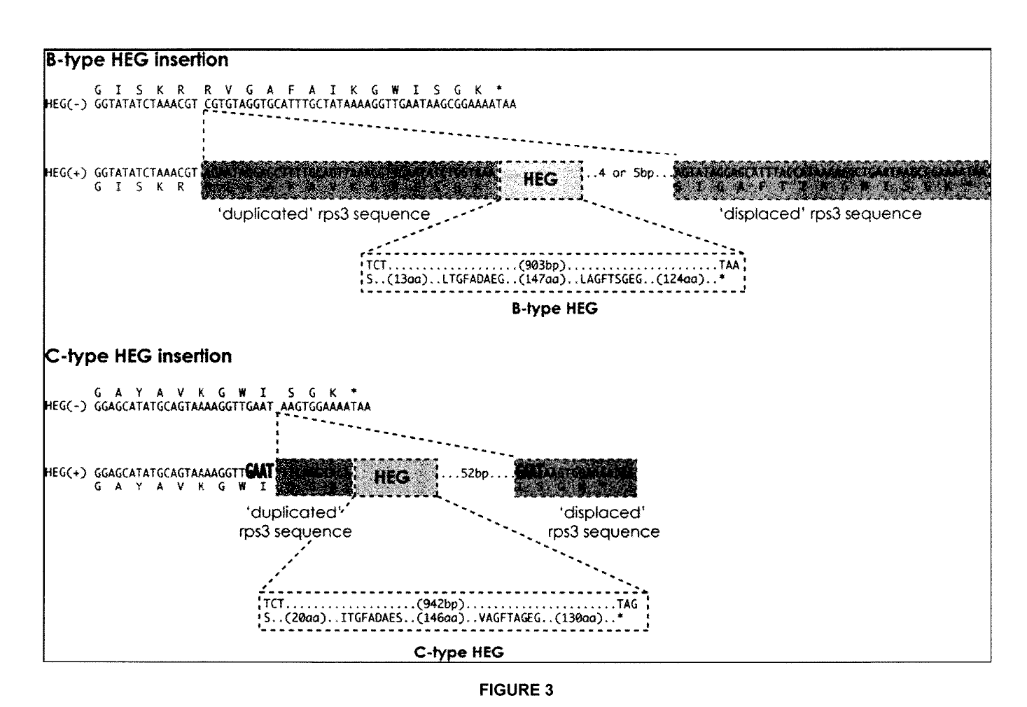

All rnl-U11 regions that yielded PCR products of ˜2.4 kb were sequenced and found to contain a group I intron-encoded RPS3 gene plus a single double-motif LAGLIDADG HEG that was inserted in one of two locations within the RPS3 C-terminal region, herein referred to as the B- and C-type HEG insertions (see FIGS. 2C and D, table 2). These examples are designated as mono-ORFic as only one RPS3-HEG fusion is present within the intron. HEG insertion point and the arrangement of the HEase coding region have been previously described for O. novo-ulmi subsp. americana (Gibb and Hausner 2005). The newly identified C-type HEG insertions identified in this study are listed in table 2. The C-type HEG insertions are associated with a short direct repeat, 5′-GAAT-3′ (table 3). In addition, 52 by separates the C-terminal (or 3′ end) of the Rps3-HEG fusion from the original RPS3 C-terminus that was displaced downstream by the insertion event; this displaced sequence is likely noncoding (FIG. 3). The source of the 52-bp segment is not known as BlastN searches yielded no significant hits. In each case, the HEG insertion event displaced the original RPS3 C-terminal coding region (see FIG. 3). However, the effect of the HEG insertion on RPS3 function is negated because the displaced RPS3-coding segment is essentially duplicated to generate a new Rps3 C-terminus. We found that 12 of 16 C-type HEGs showed evidence of degeneration caused by indels within the HEase-coding region that resulted in frameshift mutations and premature termination codons. Three strains of Ophiostoma europhioides (WIN(M) 449, 1430, and 1431), one strain of Leptographium pithyophilum, and two strains of L. truncatum (WIN(M) 254 and 1434) were noted to have a single HEG insertion, referred to as the B site that is located about 28 by upstream of the C insertion site (see FIG. 2C and table 2). The O. europhioides, L. pithyophilum, and L. truncatum sequences were compared with each other's ml U11 region including the RPS3-HEG-minus O. aureum U11 sequence. Comparative analysis showed that within this group, the HEG is inserted such that the original C-terminus (45 bp) of the resident RPS3 gene is displaced downstream from the resultant RPS3-HEG fusion. As observed for the C-type HEGs, the B-type HEG insertions are also associated with duplications of the displaced RPS3 C-terminal sequences ensuring that the RPS3-coding regions remain intact. Similar to C-type insertions, the C-terminal (or 3′ end) of the RPS3 HEG-coding region is separated from the original RPS3 C-terminus that was displaced by the insertion event (FIG. 3). However, the spacer sequence is only 4 or 5 by (FIGS. 2C and 3), as opposed to the longer 52-bp spacer associated with C-type insertions. Furthermore, the spacer sequences show no similarity to any other ml-U11 sequence, suggesting that these sequences were introduced during the HEG insertion event. For B-type insertions, three HEase ORFs appear intact, whereas four possess indels and missense mutations resulting in premature stop codons (table 2). The upstream RPS3-coding regions in all cases were always noted to be intact, that is, no premature stop codons.

TABLE 3
Sequences Upstream and Downstream of RPS3 HEG Insertions

| | Sequences Before (3′) | Sequences After (5′) | |
|---|---|---|---|
| Organism and Strain Number | the HEG Insertion Point | the HEG Insertion Point | Type |
| Ophiostoma ulmi (WIN(M) 1223) | AGGTTGAAT | GAAT.AAGTGGA | C |
| Ophiostoma novo-ulmi subsp americana | AGGTTGAAT | GAAT.AAGTGGA | C |
| (WIN(M) 900) | | | |
| Ophiostoma himal-ulmi (CBS 374.67) | AGGTTGAAT | GAAT.AAGTGGA | C |
| Sporothrix sp. (WIN(M) 924) | AGGTTGG^{a}AT | GAAT.AAGTGGA | C |
| Ophiostoma distortum (WIN(M) 847) | AGGTTGAAT | GAAT.AAGTGGA | C |
| Ophiostoma minus (WIN(M) 861) | AGGTTGGAT | GAAT.AAGTGGA | C |
| Ceratocystiopsis brevicomi (WIN(M) 1452) | AGGTTGAAT | GAAT.AAGTGGA | C |
| Ophiostoma torulosum (WIN(M) 730) | AGGTTGAAT | GAAT.AAGTGGA | C |
| Ophiostoma penicillatum (WIN(M) 27) | AGGTTGAAT | GAAT.AAGTGGA | C |
| Ceratocystis curvicollis (WIN(M) 55) | AGGATGAAT | GAAT.AAGTGGA | C |
| Ophiostoma tetropii (WIN(M) 111) | AGGTTGAAT | GAAT.AAGTGGA | C |
| O. tetropii (WIN(M) 451) | AGGTTGAAT | GAAT.AAGTGGA | C |
| Ophiostoma ips (WIN(M) 923) | TAAAAGGTT | GAAT.AATTGGA | C′ |
| Ophiostoma europhioides (WIN(M) 1431) | TCTAAACGT | AGTATAGGAGC | B |
| O. europhioides (WIN(M) 1430) | TCTAAACGT | AGTATAGGAGC | B |
| O. europhioides (WIN(M) 449) | TCTAAACGT | AGTATAGGAGC | B |
| Leptographium truncatum (WIN(M) 1434) | TCTAAACGT | AGTATAGGAGC | B |
| L. truncatum (WIN(M) 254) | TCTAAACGT | AGTATAGGAGC | B |
| Leptographium pithyophilum (WIN(M) 1454) | TCTAAACGT | AGTATAGGAGC | B |
| Ophiostoma laricis (WIN(M) 1461) | TCTAAACGT | AGTATAGGAGC | B |
| Ophiostoma piceaperdum (WIN(M) 979) | AATTTTCCT | GTATATGAC | A |
| Ophiostoma laricis (WIN(M) 1461) | AATTTTCCT | GTATATGAC | A |

^{a}Nucleotides shown in bold indicate positions that deviate from the consensus sequence 3′ to HEG insertion sites.

### Independent Insertion of Two LAGLIDADG HEGs in a Single RPS3 Gene

A variation of the O. piceaperdum mL2449 intron ORF arrangement was noted in a strain of O. laricis (WIN(M) 1461) (FIG. 2E). Here, the resident RPS3-coding region was invaded independently by two double-motif LAGLIDADG-type HEGs, creating two hybrid fusion ORFs. One HEG insertion is an A-type insertion, where the HEG is fused in-frame to the N-terminus of the original RPS3 ORF. The second HEG insertion is a B-type insertion, where the HEG is fused in-frame to the C-terminus of the RPS3-coding region. However, both HEGs are characterized by frameshift mutations, suggesting that they have degenerated. In both Rps3-HEG fusions, the RPS3-coding regions are upstream of the HEase-coding segments, implying that frameshift mutations within the HEGs should not directly affect the translation of Rps3. The two Rps3-HEG fusion ORFs are separated by a 36-bp sequence that lacks similarity to U11 region/intron sequence, and the second ORF starts with a 38-bp segment that may represent a new Rps3 N-terminus, similar to the situation described for A-type insertions in O. piceaperdum (see FIG. 2B). In summary, the resident RPS3 gene has essentially been split such that the N- and C-termini are now components of two ORFs that each includes a LAGLIDADG HEase.

### Phylogenetic Analysis of the LAGLIDADG HEGs Inserted in RPS3 Genes

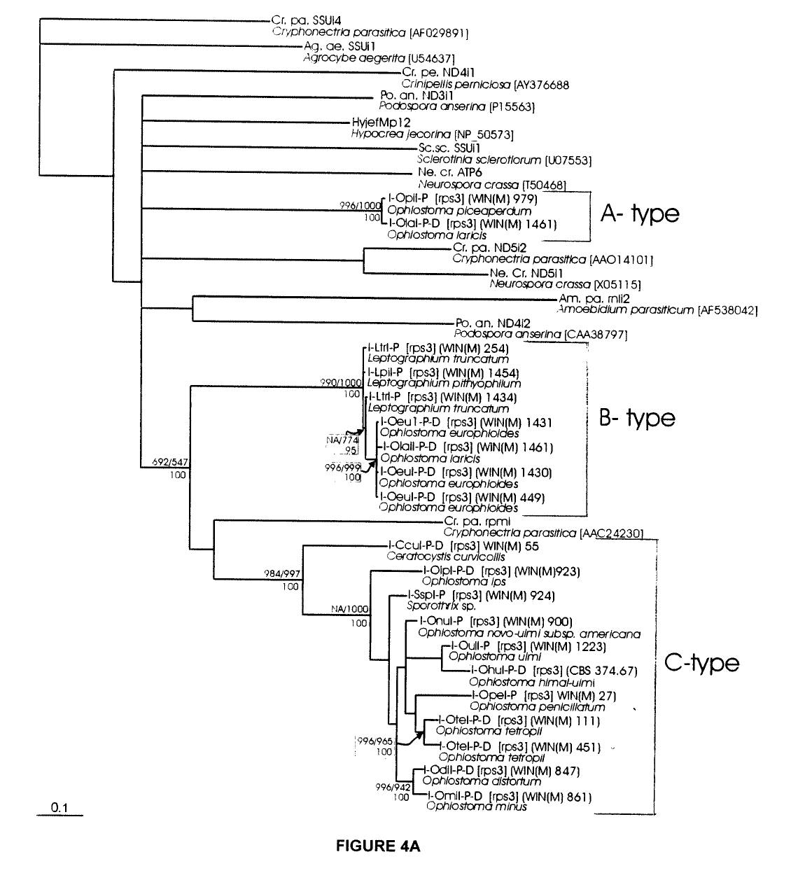

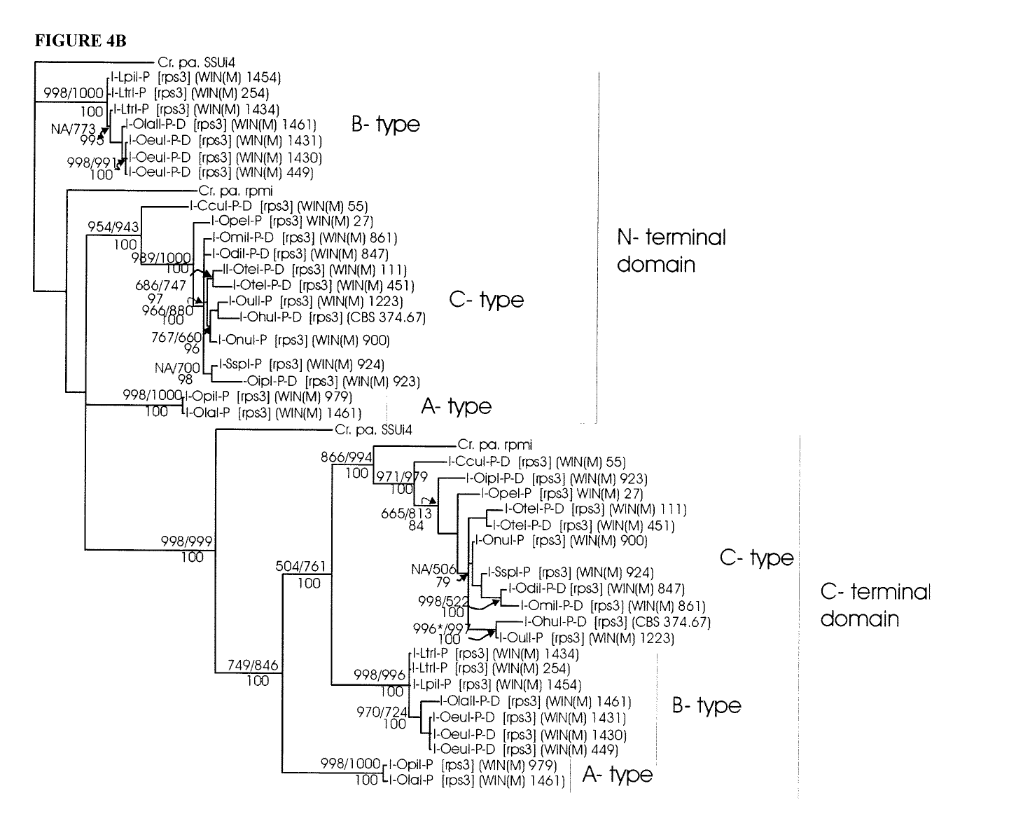

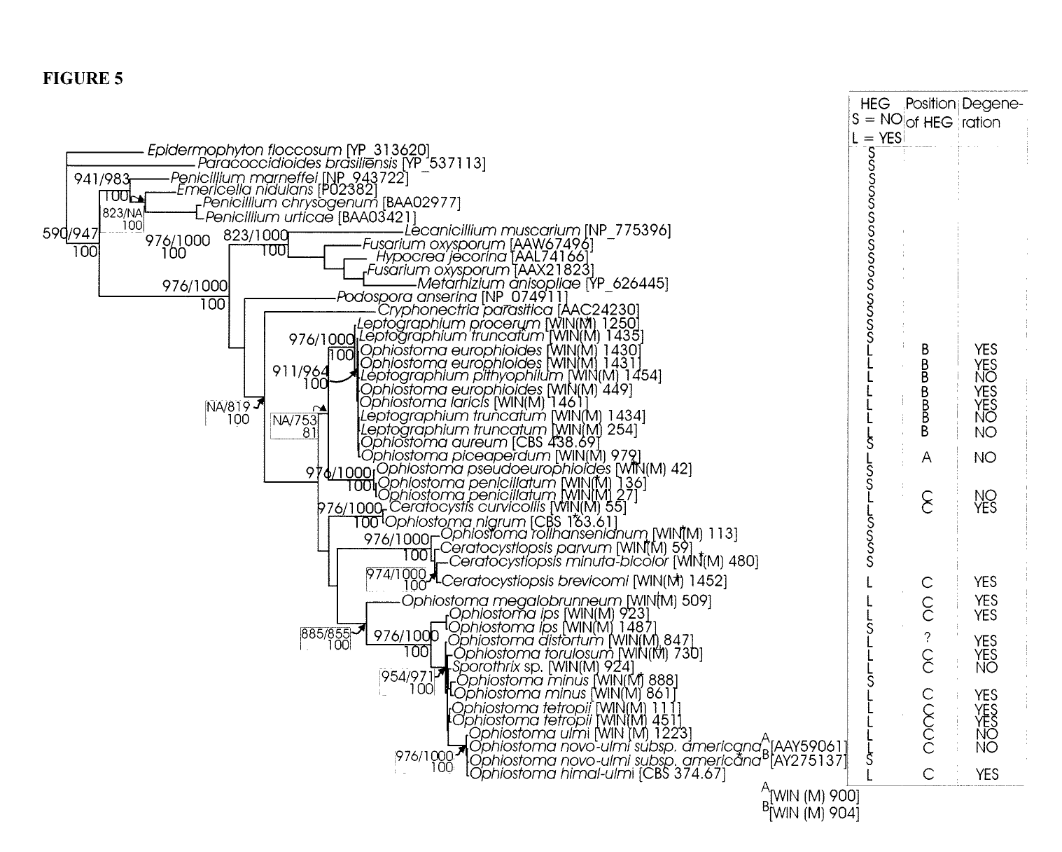

A BlastP search identified double-motif LAGLIDADG HEases related to those we identified in this study. To analyze the evolutionary relationships among the HEGs, the sequences were combined into a single alignment and analyzed by a variety of phylogenetic methods (FIGS. 4A and B). Phylogenetic analyses yielded evolutionary trees that grouped the N- and C-terminal sequences into separate clades (FIG. 4B). This tree topology suggests that the two halves of the LAGLIDADG sequences originated by a gene duplication event (Haugen and Bhattacharya 2004). When the HEGs were treated as a continuous sequence; they grouped into three distinct clades (FIG. 4A). Both phylogenetic analyses suggest that the C-terminally inserted HEGs (sites B and C) share a recent common ancestor and are distantly related to the A type HEG that inserted in the N-terminus of RPS3 gene. Group I intron-encoded LAGLIDADG ORFs recovered from Genbank by BlastP analysis failed to identify a potential intron-encoded ancestor for the RPS3 HEGs discovered in this study, whereas the previously described HEG inserted within the C. parasitica RPS3 gene appears to be related to the C-type HEGs identified in species of Ophiostoma (including Leptographium) species.

### Example 5

### Phylogenetic Analysis of the RPS3 Host Gene

The RPS3 Host Gene Phylogeny Suggests Vertical rather than Horizontal Inheritance

To determine the phylogenetic relationship among the host RPS3 genes, and to test for horizontal transfer of RPS3 and HEG genes, we extracted related RPS3 sequences from GenBank representing two major groups within the Pezizomycotina: the Eurotiomycetes and the Sordariomycetes (Blackwell et al. 2006). In total, 47 RPS3 sequences were compiled of which our study generated 33 new RPS3 sequences for meiotic and mitotic members of the genus Ophiostoma sensu lato. The phylogenetic analysis of the RPS3 data yielded the tree shown in FIG. 5. Although RPS3 is encoded within a potentially mobile group I intron, and in some instances the RPS3 ORF is associated with potentially mobile HEGs, the comparison between the RPS3 and the HEG trees provides no evidence that the RPS3 gene has been transferred horizontally. Comparative phylogenetic analysis of RPS3 sequences with their corresponding HEGs failed to show evidence for recent lateral transfers of either the HEG or RPS3 sequences, as the phylogenetic trees observed appeared to be congruent for both the RPS3- and HEase-coding regions.

### Example 6

### Recognition Site Cleavage

I-OnuI and I-LtrI are Functional LAGLIDADG Enzymes that Cleave at or Near the HEG Insertion

### Site

Phylogenetic analysis showed that the B- and C-type RPS3 HEGs may share a common ancestor. We focused on two HEG insertions, a B-type HEG in the RPS3 gene of L. truncatum strain WIN(M) 254 and a C-type HEG in the RPS3 gene of O. novo-ulmi subsp. americana strain WIN(M)900. Comparative sequence analysis suggested that for the C-type RPS3 insertion, a GAAT sequence would be a logical candidate as a cleavage and insertion site (Gibb and Hausner 2005). For the B-type RPS3 insertions, potential cleavage-insertion sites were not apparent; thus, the HEase was characterized with regard to its cleavage site within the RPS3 gene. The cleavage site assays also determined whether the LAGLIDADG HEases inserted within the C-terminus of the RPS3 gene are functional.

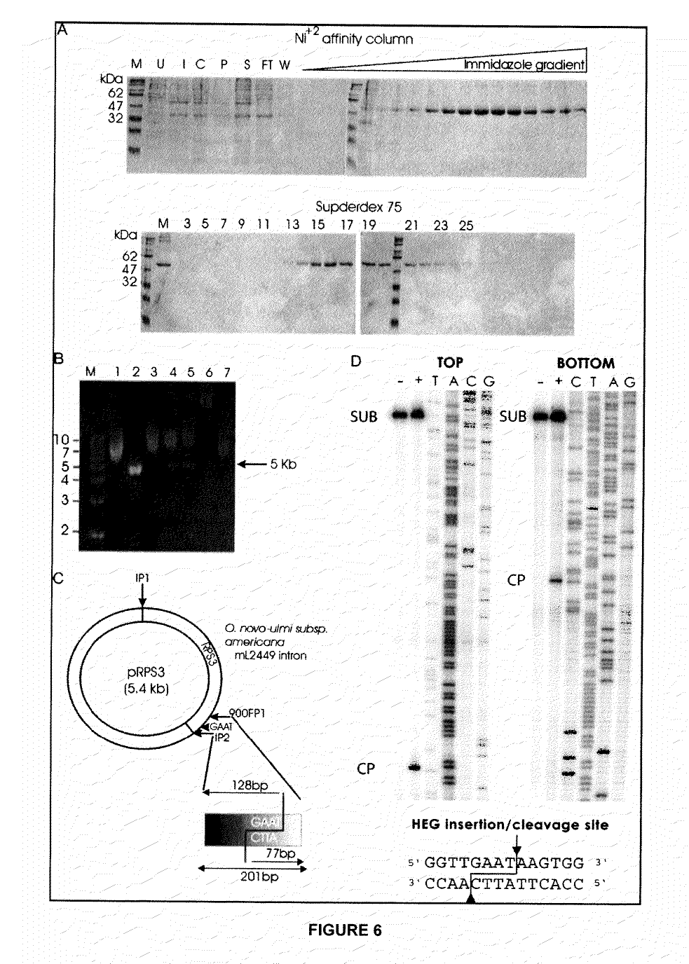

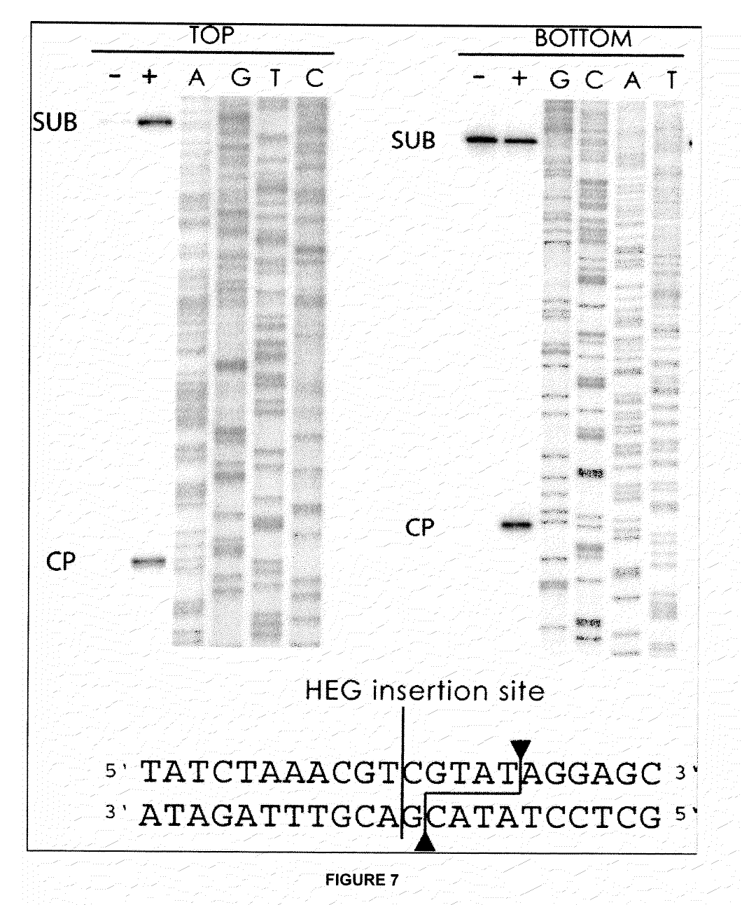

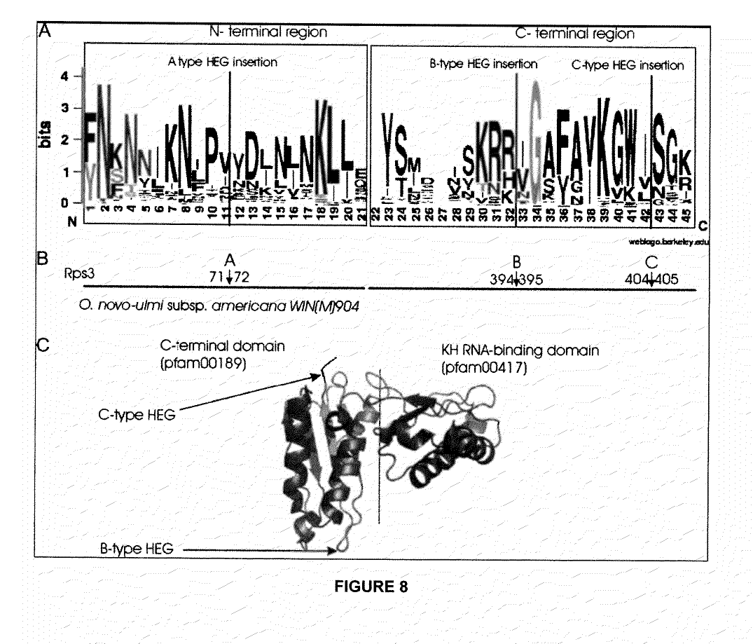

In order to characterize each HEase, we initially synthesized two gene constructs for each HEase for use in overexpression studies. One construct included the entire RPS3-HEG fusion, whereas a second construct corresponded to the LAGLIDADG endonuclease portion of the RPS3-HEG fusion. In each case, the genetic code was optimized for expression in E. coli. Although both proteins expressed well, the Rps3-HEG fusion did not bind to nickel-charged resin, whereas the HEG-only construct was readily purified by nickel-affinity and gel-filtration chromatography (FIG. 6A). For the C-type HEG, purified HEase was incubated with plasmid substrate (pRPS3) containing a cloned RPS3-HEG-minus allele (source: O. novo-ulmi subsp. americana strain WIN(M) 904). As shown in FIG. 6B, circular pRPS3 was linearized after addition of the purified HEase (FIG. 6B, lanes 3-5). In contrast, no cleavage was observed by the HEase with a substrate that corresponded to HEG-plus allele (pRPS3/HEG), or a substrate containing a different group I intron-encoded ORF (mL1699 ORF; -pU7-1409) (FIG. 6B). In accordance with standard nomenclature for HEases, we have named the endonuclease I-OnuI. The I-OnuI cleavage sites were mapped by incubating the enzyme with end-labeled substrate that included the predicted I-OnuI insertion site. By resolving the cleavage products next to corresponding DNA sequencing ladders, the I-OnuI cleavage site was mapped to positions 1214 and 1210 on the coding and noncoding strands, respectively, of the O. novo-ulmi subsp. americana (WIN(M) 904) RPS3 gene (FIGS. 6C and D). These nucleotide positions correspond to the 5′-GAAT-3′ sequence previously noted to form a 4-bp direct repeat flanking the HEG insertion site (FIGS. 3 and 6D, table 3). Similarly, the I-LtrI cleavage sites were mapped as for I-OnuI, except the cleavage site substrate was derived from an RPS3-minus HEG allele obtained from L. truncatum strain WIN(M)1435. For I-LtrI, the data show that the HEase generated a 3′ 4 nt overhang (GTAT; FIG. 7). Based on comparative sequence analysis, the insertion site for I-LtrI is 1 bp upstream from the 4-bp cleavage site, that is, 5′ . . . GT[HEG]C↑GTAT↓AGGA . . . 3′, where ↑ and ↓ denotes the bottom- and top-strand cleavage sites, respectively (see FIG. 7).

All citations are herein incorporated by reference, as if each individual publication was specifically and individually indicated to be incorporated by reference herein and as though it were fully set forth herein. Citation of references herein is not to be construed nor considered as an admission that such references are prior art to the present invention.

The invention includes all embodiments, modifications and variations substantially as hereinbefore described and with reference to the examples and figures. It will be apparent to persons skilled in the art that a number of variations and modifications can be made without departing from to the scope of the invention as defined in the claims. Examples of such modifications include the substitution of known equivalents for any aspect of the invention in order to achieve the same result in substantially the same way.

### REFERENCES

Abu-Amero S N, Charter N W, Buck K W, Brasier C M. 1995.Nucleotide-sequence analysis indicates that a DNA plasmid in a diseased isolate of Ophiostoma novo-ulmi is derived by recombination between two long repeat sequences in the mitochondrial large subunit ribosomal RNA gene. Curr Genet. 28:54-59.

Altschul S F, Gish W, Miller W, Myers E W, Lipman D J. 1990. Basic local alignment search tool. J Mol Biol. 215:403-410.

Altschul et al. 1990, J Mol. Biol. 215: 403-410 and ALTSCHUL et al. (1997), Nucleic Acids Res. 25: 3389-3402.

Ausubel et al., Current Protocols in Molecular Biology, Greene Publishing Associates and John Wiley & Sons, NY, 1994

Arlt H, Steglich G, Perryman R, Guiard B, Neupert W, Langer T. 1998. The formation of respiratory chain complexes in mitochondria is under the proteolytic control of the m-AAA protease. EMBO J. 17:4837-4847.

Belcour L, Rossignol M, Koll F, Sellem C H, Oldani C. 1997. Plasticity of the mitochondrial genome in Podospora. Polymorphism for 15 optional sequences: group-I, group-II introns, intronic ORFs and an intergenic region. Curr Genet. 31:308-317.

Belfort M. 2003. Two for the price of one: a bifunctional intronencoded DNA endonuclease-RNA maturase. Genes Dev. 17:2860-2863.

Belfort M, Derbyshire V, Parker M M, Cousineau B, Lambowitz A M. 2002. Mobile introns: pathways and proteins. In: Craig N L, Craigie R, Gellert M, Lambowitz A M, editors. Mobile DNA II. Washington (D.C.): American Society of Microbiology Press. p. 761-783.

Belfort M, Perlman P S. 1995. Mechanisms of intron mobility. J Biol Chem. 270:30237-30240.

Belfort M, Roberts R J. 1997. Homing endonucleases: keeping the house in order. Nucleic Acids Res. 25:3379-3388.

Bell J A, Monteiro-Vitorello C B, Hausner G, Fulbright D W, Bertrand H. 1996. Physical and genetic map of the mitochondrial genome of Cryphonectria parasitica Ep155. Curr Genet. 30:34-43.

Blackwell M, Hibbett D S, Taylor J W, Spatafora J W. 2006. Research coordination networks: a phylogeny for kingdom fungi (deep Hypha). Mycologia. 98:829-837.

Bonen L, Calixte S. 2006. Comparative analysis of bacterialorigin genes for plant mitochondrial ribosomal proteins. Mol Biol Evol. 23:701-712.

Bonocora R P, Shub D A. 2001. A novel group I intron-encoded endonuclease specific for the anticodon region of tRNA(fMet) genes. Mol Microbiol. 39:1299-1306.

Bullerwell C E, Burger G, Lang B F. 2000. A novel motif for identifying rps3 homologs in fungal mitochondrial genomes. Trends Biochem Sci. 25:363-365.

Bullerwell C E, Leigh J, Seif E, Longcore J E, Lang B F. 2003. Evolution of the fungi and their mitochondrial genomes. In: Arora D K, Khachatourians G G, editors. Applied mycology and biotechnology, Vol. III: Fungal genomics. New York: Elsevier Science. p. 133-159.

Burke J M, RajBhandary U L. 1982. Intron within the large rRNA gene of N. crassa mitochondria: a long open reading frame and a consensus sequence possibly important in splicing. Cell. 31:509-520.

Caprara M G, Waring R B. 2005. Group I introns and their maturases: uninvited, but welcome guests. Nucl Acids Mol Biol. 16:103-119.

Chan et al., Fmoc Solid Phase Peptide Synthesis, Oxford University Press, Oxford, United Kingdom, 2005;

Chevalier B S, Stoddard B L. 2001. Homing endonucleases: structural and functional insight into the catalysts of intron/intein mobility. Nucleic Acids Res. 29:3757-3774.

Cho T, Palmer J D. 1999. Multiple acquisitions via horizontal transfer of a group I intron in the mitochondrial cox1 gene during evolution of the Araceae family. Mol Biol Evol. 16:1155-1165.

Clark-Walker G D. 1992. Evolution of mitochondrial genomes in fungi. Int Rev Cytol. 141:89-127.

Crooks G E, Hon G, Chandonia J M, Brenner S E. 2004. WebLogo: a sequence logo generator. Genome Res. 14:1188-1190.

Cummings D J, Domenico J M, Nelson J. 1989. DNA sequence and secondary structures of the large subunit rRNA coding regions and its two class I introns of mitochondrial DNA from Podospora anserina. J Mol Evol. 28:242-255.

Cummings D J, McNally K L, Domenico J M, Matsuura E T. 1990. The complete DNA sequence of the mitochondrial genome of Podospora anserina. Curr Genet. 17:375-402.

Cummings D J, Turker M S, Domenico J M. 1986. Mitochondrial excision-amplification plasmids in senescent and long-lived cultures of Podospora anserina. In: Wickner R B, Hinnebusch A,

Lambowitz A M, Gonsalus I C, Hollaender A, editors. Extrachromosomal elements in lower eukoryotes. New York: Plenum Press. p. 129-146.

Dayhoff M O, Schwartz R M, Orcutt B C. 1978. A model of evolutionary change in proteins. In:

Dayhoff M O, editor. Atlas of protein sequence and structure. Washington (D.C.): National Biomedical Research Foundation. Suppl. 3:p. 345-352.

Dujon B. 1989. Group I introns as mobile genetic elements: facts and mechanistic speculations—a review. Gene. 82:91-114.

Dujon B, Belcour L. 1989. Mitochondrial DNA instabilities and rearrangements in yeasts and fungi. In: Berg D E, Howe M M, editors. Mobile DNA. Washington (D.C.): American Society of Microbiology. p. 861-878.

Felsenstein J. 1985. Confidence limits on phylogenies: an approach using the bootstrap. Evolution. 39:783-791.

Felsenstein J. 1989. PHYLIP-Phylogeny Inference Package (Version 3.2). Cladistics. 5:164-166.

Felsenstein J. 2005. PHYLIP (Phylogeny Inference Package) version 3.6. Distributed by the author. Seattle (Wash.): Department of Genome Sciences, University of Washington.

Gibb E A, Hausner G. 2005. Optional mitochondrial introns and evidence for a homing-endonuclease gene in the mtDNA nil gene in Ophiostoma ulmi s. lat. Mycol Res. 109:1112-1126.

Gillha N W, Boynton J E, Hauser C R. 1994. Translational regulation of gene expression in chloroplasts and mitochondria. Annu Rev Genet. 28:71-93.

Gimble F S. 2000. Invasion of a multitude of genetic niches by mobile endonuclease genes. FEMS Microbiol Lett. 185:99-107.

Gobbi E, Firm G, Carpanelli A, Locci R, Van Alfen N K. 2003. Mapping and characterization of polymorphism in mtDNA of Cryphonectria parasitica: evidence of the presence of an optional intron. Fungal Genet Biol. 40:215-224.

Goddard M R, Burt A. 1999. Recurrent invasion and extinction of a selfish gene. Proc Natl Acad Sci USA. 96:13880-13885.

Gogarten J P, Hilario E. 2006. Inteins, introns, and homing endonucleases: recent revelations about the life cycle of parasitic genetic elements. BMC Evol Biol. 6:94. doi:10.1186/1471-2148-6-94.

Gonzalez P, Barroso G, Labarere J. 1998. Molecular analysis of the split cox1 gene from the Basidiomycota Agrocybe aegerita: relationship of its introns with homologous Ascomycota introns and divergence levels from common ancestral copies. Gene. 220:45-53.

Guhan N, Muniyappa K. 2003. Structural and functional characteristics of homing endonucleases. Crit Rev Biochem Mol Biol. 38:199-248.

Haugen P, Bhattacharya D. 2004. The spread of LAGLIDADG homing endonuclease genes in rDNA. Nucleic Acids Res. 32:2049-2057.

Haugen P, Runge H J, Bhattacharya D. 2004. Long-term evolution of the 5788 fungal nuclear small subunit rRNA group I introns. RNA. 10:1084-1096.

Haugen P, Simon D M, Bhattacharya D. 2005. The natural history of group I introns. Trends Genet. 21:111-119.

Hausner G. 2003. Fungal mitochondrial genomes, plasmids and introns. In: Arora D K, Khachatourians G G, editors. Applied mycology and biotechnology, Vol. III: fungal genomics. New York: Elsevier Science. p. 101-131.

Hausner G, Monteiro-Vitorello C B, Searles D B, Maland M, Fulbright D W, Bertrand H. 1999. A long open reading frame in the mitochondrial LSU rRNA group-I intron of Cryphonectria parasitica encodes a putative S5 ribosomal protein fused to a maturase. Curr Genet. 35:109-117.

Hausner G, Reid J. 2003. Notes on Ceratocystis brunnea and Ophiostoma based on partial ribosomal DNA sequence data. Can J Bot. 81:865-876.

Hausner G, Reid J, Klassen G R. 1992. Do galeate-ascospore members of the Cephaloascaceae, Endomycetaceae and Ophiostomataceae share a common phylogeny? Mycologia. 84:870-881.

Hausner G, Reid J, Klassen G R. 1993. On the phylogeny of Ophiostoma, Ceratocystis s.s., Microascus, and relationships within Ophiostoma based on partial ribosomal DNA sequences. Can J Bot. 71:1249-1265.

Hausner G, Reid J, Klassen G R. 2000. On the phylogeny of the members of Ceratocystis s.l. that possess different anamorphic states, with emphasis on the asexual genus Leptographium, based on partial ribosomal sequences. Can J Bot. 78:903-916.

Iwamoto M, Pi M, Kurihara M, Morio T, Tanaka Y. 1998. A ribosomal protein gene cluster is encoded in the mitochondrial DNA of Dictyostelium discoideum: UGA termination codons and similarity of gene order to Acanthamoeba castellanii. Curr Genet. 33:304-310.

Johansen S, Haugen P. 2001. A new nomenclature of group I introns in ribosomal DNA. RNA. 7:935-936.

Johansen S D, Haugen P, Nielsen H. 2007. Expression of protein coding genes embedded in ribosomal DNA. Biol Chem. 388:679-686.

Jurica M S, Stoddard B L. 1999. Homing endonucleases: structure, function and evolution. Cell Mol Life Sci. 55:1304-1326.

Kubelik A R, Kennell J C, Akins R A, Lambowitz A M. 1990. Identification of Neurospora mitochondrial promoters and analysis of synthesis of the mitochondrial small rRNA in wild-type and the promoter mutant [poky]. J Biol Chem. 265:4515-4526.

Lambowitz A M, Caprara M G, Zimmerly S, Perlman P S. 1999. Group I and group II ribozymes as RNPs: clues to the past and guides to the future. In: Gesteland R F, Cech T R, Atkins J F, editors. The RNA world. New York: Cold Spring Harbor Laboratory Press. p. 451-485.

Lambowitz A M, Perlman P S. 1990. Involvement of aminoacyl tRNA synthetases and other proteins in group I and group II intron splicing. Trends Biochem Sci. 15:440-444.

LaPolla R J, Lambowitz A M.1981. Mitochondrial ribosomeassembly in Neurospora crassa. Purification of the mitochondrially synthesized ribosomal protein, S-5. J Biol Chem. 256:7064-7067.

Laroche J, Bousquet J. 1999. Evolution of the mitochondrial rps3 intron in perennial and annual angiosperms and homology to nad5 intron 1. Mol Biol Evol. 16:441-452.

Mota E M, Collins R A. 1988. Independent evolution of structural and coding regions in a Neurospora mitochondrial intron. Nature. 332:654-656.

Nicholas K B, Nicholas H B Jr, Deerfield D W. 1997. GeneDoc: analysis and visualization of genetic variation.EMBNEW NEWS.4:14.

Page R D. 1996. TreeView: an application to display phylogenetic trees on personal computers. Comput Appl Biosci. 12:357-358.

Paquin B, Laforest M J, Lang B F. 1994. Interspecific transfer of mitochondrial genes in fungi and creation of a homologous hybrid gene. Proc Natl Acad Sci USA. 91:11807-11810.

Paquin B, Lang B F. 1996. The mitochondrial DNA of Allomyces macrogynus: the complete genomic sequence from an ancestral fungus. J Mol Biol. 255:688-701.

Peptide and Protein Drug Analysis, ed. Reid, R., Marcel Dekker, Inc., 2000;

Ronquist F. 2004. Bayesian inference of character evolution. Trends Ecol Evol. 19:475-481.

Ronquist F, Huelsenbeck J P. 2003. MrBayes 3: Bayesian phylogenetic inference under mixed models. Bioinformatics. 19:1572-1574.

Salvo J L, Rodeghier B, Rubin A, Troischt T. 1998. Optional introns in mitochondrial DNA of Podospora anserina are the primary source of observed size polymorphisms. Fungal Genet Biol. 23:162-168.

Sambrook et al., Molecular Cloning: A Laboratory Manual, 3^{rd} ed., Cold Spring Harbor Press, Cold Spring Harbor, N.Y. 2001;

Schaefer B. 2003. Genetic conservation versus variability in mitochondria: the architecture of the mitochondrial genome in the petite-negative yeast Schizosaccharomyces pombe. Curr Genet. 43:311-326.

Schluenzen F, Tocilj A, Zarivach R, et al. (11 co-authors). 2000. Structure of functionally activated small ribosomal subunit at 3.3 angstroms resolution. Cell. 102:615-623.

Schneider T D, Stephens R M. 1990. Sequence logos: a new way to display consensus sequences. Nucleic Acids Res. 18: 6097-6100.

Seif E, Leigh J, Liu Y, Roewer I, Forget L, Lang B F. 2005. Comparative mitochondrial genomics in zygomycetes: bacteria like RNase P RNAs, mobile elements and a close source of the group I intron invasion in angiosperms. Nucleic Acids Res. 33:734-744.

Sellem C H, Belcour L. 1994. The in vivo use of alternate 3#-splice sites in group I introns. Nucleic Acids Res. 22:1135-1137.

Sellem C H, Belcour L. 1997. Intron open reading frames as mobile elements and evolution of a group I intron. Mol Biol Evol. 14:518-526.

Sellem C H, d'Aubenton-Carafa Y, Rossignol M, Belcour L. 1996. Mitochondrial intronic open reading frames in Podospora: mobility and consecutive exonic sequence variations. Genetics. 143:777-788.

Sethuraman J, Okoli C V, Majer A, Corkery T L, Hausner G. 2008. The sporadic occurrence of a group I intron-like element in the mtDNA ml gene of Ophiostoma novo-ulmi subsp. americana. Mycol Res. 112:564-582.

Stoddard B L. 2005. Homing endonuclease structure and function. Q Rev Biophys. 38:49-95. Toor N, Zimmerly S. 2002. Identification of a family of group II introns encoding LAGLIDADG ORFs typical of group I introns. RNA. 8:1373-1377.

Upadhyay H P. 1981. A Monograph on Ceratocystis and Ceratocystiopsis. Athens: University of Georgia Press. p. 176.

Van Dyck L, Neupert W, Langer T. 1998. The ATP-dependent PIM1 protease is required for the expression of intron containing genes in mitochondria. Genes Dev. 12:1515-1524.

Wilson D N, Nierhaus K H. 2005. Ribosomal proteins in the spotlight. Crit Rev Biochem Mol Biol. 40:243-267.

Wingfield M J, Seifert K A, Webber J F. 1993. In: Wingfield M J, Seifert K A, Webber J F, editors. Ceratocystis and Ophiostoma Biology, taxonomy and ecology. American Phytopathological Society Press.ISBN 0-89054-156-6.

Epitope Mapping, ed. Westwood et al., Oxford University Press, Oxford, United Kingdom, 2000;

Zhao L, Bonocora R P, Shub D A, Stoddard B L. 2007. The restriction fold turns to the dark side: a bacterial homing endonuclease with a PD-(D/E)-XK motif. EMBO J. 26:2432-2442.

Zhu H, Macreadie I G, Buttow R A. 1987. RNA processing and expression of an intron-encoded protein in yeast mitochondria: role of a conserved docecamer sequence. Mol Cell Biol. 7:2530-2537.

1. An endonuclease comprising a polypeptide comprising the sequence set forth in SEQ ID NO:1; SEQ ID NO:35, an active fragment thereof, or sequence substantially identical thereto.

2. A nucleic acid encoding the polypeptide of claim 1.

3. The nucleic acid of claim 2 wherein the nucleic acid comprises the sequence set forth in SEQ ID NO: 2; SEQ ID NO: 36 or a sequence substantially identical thereto.

4. A nucleic acid comprising a homing endonuclease recognition site capable of being cleaved by the endonuclease of claim 1.

5. The nucleic acid of claim 4 wherein the recognition site comprises the sequence set forth in SEQ ID NO: 21 or a sequence substantially identical thereto.

6. A vector comprising the nucleic acid of claim 2.

7. The vector of claim 6 wherein the vector is an expression vector comprising a promoter operatively linked to the nucleic acid.

8. The vector of claim 7 wherein the vector comprises the sequence set forth in SEQ ID NO: 36 or a sequence substantially identical thereto.

9. A cell comprising the vector of claim 6.

10. A cell comprising the expression vector of claim 7.

11. A vector comprising the nucleic acid comprising the homing endonuclease recognition site of claim 4.

12. A cell comprising the vector of claim 11.

13. A cell comprising the homing endonuclease recognition site of claim 4, wherein the recognition site is located on a chromosome of the cell.

14. A method of producing an endonuclease comprising culturing the cell of claim 10 under conditions suitable for expression of the endonuclease polypeptide.

15. A kit comprising the nucleic acid of claim 2.

16. A kit comprising the nucleic acid of claim 4.

17. An endonucleases comprising a polypeptide comprising the sequence set forth in SEQ ID NO:
13; SEQ ID NO: 33, an active fragment thereof, or a sequence substantially identical thereto.

18. A nucleic acid encoding the polypeptide of claim 17.

19. The nucleic acid of claim 18 wherein the nucleic acid comprises the sequence set forth in SEQ ID NO:14; SEQ ID NO: 34, or a sequence substantially identical thereto.

20. A nucleic acid comprising an endonuclease recognition site capable of being cleaved by the endonuclease of claim 17.

21. The nucleic acid of claim 20 wherein the recognition site comprises the sequence set forth in SEQ ID NO: 22 or a sequence substantially identical thereto.

22. A vector comprising the nucleic acid of claim 18.

23. The vector of claim 22 wherein the vector is an expression vector comprising a promoter operatively linked to the nucleic acid.

24. The vector of claim 23 wherein the vector comprises the sequence set forth in SEQ ID NO: 34 or a sequence substantially identical thereto.

25. A cell comprising the vector of claim 22.

26. A cell comprising the expression vector of claim 23.

27. A vector comprising the nucleic acid comprising the homing endonuclease recognition site of claim 20.

28. A cell comprising the vector of claim 27.

29. A cell comprising the homing endonuclease recognition site of claim 20, wherein the recognition site is located on a chromosome of the cell.

30. A method of producing an endonuclease comprising culturing the cell of claim 26 under conditions suitable for expression of the endonuclease polypeptide.

31. A kit comprising the nucleic acid of claim 18.

32. A kit comprising the nucleic acid of claim 20.

33. A polypeptide comprising one or more sequences selected from the group consisting of SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 33, SEQ ID NO: 35, or a sequence substantially identical thereto.

34. A nucleic acid comprising one or more sequences selected from the group consisting of SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 34, SEQ ID NO: 36, or a sequence substantially identical thereto.

35. A nucleic acid comprising one or more sequences selected from the group consisting of SEQ ID NO: 34, SEQ ID NO: 36, or a sequence substantially identical thereto.

36. A vector comprising the nucleic acid of claim 34.

37. A vector comprising the nucleic acid of claim 35.

38. The vector of claim 36 wherein the vector is an expression vector comprising a promoter operatively linked to the nucleic acid.

39. A nucleic acid comprising a homing endonuclease recognition site comprising one or more sequences selected from the group consisting of SEQ ID NO: 17, SEQ ID NO: 18, SEQ ID NO:
19, SEQ ID NO: 20, SEQ ID NO:21 and SEQ ID NO: 22, or a sequence substantially identical thereto.

40. A vector comprising the nucleic acid of claim 39.
